SEQUENCE LISTING (115 sequences)

SEQ ID NO: 1 - protein, 188 aa, Woodchuck hepatitis virus
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 2 - protein, 149 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVI

SEQ ID NO: 3 - protein, 24 aa, Artificial Sequence
  Synthesized Construct
NSELLSLINDMPITNDQKKLMSNN

SEQ ID NO: 4 - DNA, 3323 bp, Woodchuck hepatitis virus
aattcgggacataccacgtggtttagttccgcctcaaactccaacaaatcgagatcaagggagaaagcctactcctccaactccacctctaagagatactcacccccacttaactatgaaaaatcagacttttcatctccaggggttcgtagacggattacgagacttgacaacaacggaacgccaacacaatgcctatggagatccttttacaacactaagccctgcggttcctactgtatccaccatattgtctcctccctcgacgactggggaccctgcactgtcaccggagatgtcaccatcaagtctcctaggactcctcgcaggattacaggtggtgtatttcttgtggacaaaaatcctaacaatagctcagaatctagattggtggtggacttctctcagttttccagggggcataccagagtgcactggccaaaattcgcagttccaaacttgcaaacacttgccaacctcctgtccaccaacttgcaatggctttcgttggatgtatctgcggcgttttatcatatacctattagtcctgctgctgtgcctcatcttcttgttggttctcctggactggaaaggtttaatacctgtctgtcctcttcaacccacaacagaaacaacagtcaattgcagacaatgcacaatctctgcacaagacatgtatactcctccttactgttgttgtttaaaacctacggcaggaaattgcacttgttggcccatcccttcatcatgggctttaggaaattacctatgggagtgggccttagcccgtttctcttggctcaatttactagtgcccttgcttcaatggttaggaggaatttccctcattgcgtggtttttgcttatatggatgatttggttttgggggcccgcacttctgagcatcttaccgccatttattcccatatttgttctgtttttcttgatttgggtatacatttgaatgtcaataaaacaaaatggtggggcaatcatctacatttcatgggatatgtgattactagttcaggtgtattgccacaagacaaacatgttaagaaaatttcccgttatttgcgctctgttcctgttaatcaacctctggattacaaaatttgtgaaagattgactggtattcttaactatgttgctccttttacgctatgtggatacgctgctttaatgcctttgtatcatgctattacttcccgtacggctttcattttctcctccttgtataaatcctggttgctgtctctttatgaggagttgtggcccgttgtcaggcaacgtggcgtggtgtgcactgtgtttgctgacgcaacccccactggttggggcattgccaccacctatcaactcctttccgggactttcgctttccccctccctattgccacggcggaactcattgccgcctgccttgcccgctgctggacaggggctcggctgttgggcactgacaattccgtggtgttgtcggggaagctgacgtcctttccatggctgctcgcctgtgttgccaactggattctgcgcgggacgtccttctgctacgtcccttcggccctcaatccagcggaccttccttcccgcggcctgctgccggttctgcggcctcttccgcgtcttcgccttcgccctcagacgagtcggatctccctttgggccgcctccccgcctgtttcgcctcggcgtccggtccgtgttgcttggtcttcacctgtgcagaattgcgaaccatggattccaccgtgaactttgtctcctggcatgcaaatcgtcaacttggcatgccaagtaaggacctttggactccttatataaaagatcaattattaactaaatgggaggagggcagcattgatcctagattatcaatatttgtattaggaggctgtaggcataaatgcatgcgacttctgtaaccatgtatctttttcacctgtgccttgtttttgcctgtgttccatgtcctacttttcaagcctccaagctgtgccttggatggctttggggcatggacatagatccctataaagaatttggttcatcttatcagttgttgaattttcttcctttggacttctttcctgaccttaatgctttggtggacactgctactgccttgtatgaagaagagctaacaggtagggaacattgctctccgcaccatacagctattagacaagctttagtatgctgggatgaattaactaaattgatagcttggatgagctctaacataacttctgaacaagtaagaacaatcatagtaaatcatgtcaatgatacctggggacttaaggtgagacaaagtttatggtttcatttgtcatgtctcacttttggacaacatacagttcaagaatttttagtaagttttggagtatggatcagaactccagctccatatagacctcctaatgcacccattctctcgactcttccggaacatacagtcattaggagaagaggaggtgcaagagcttctaggtcccccagaagacgcactccctctcctcgcaggagaagatctcaatcaccgcgtcgcagacgctctcaatctccatctgccaactgctgatcttcaatgggtacataaaactaatgctattacaggtctttactctaaccaagctgctcagttcaatccgcattggattcaacctgagtttcctgaacttcatttacataatgatttaattcaaaaattgcaacagtattttggtcctttgactataaatgaaaagagaaaattgcaattaaattttcctgccagatttttccccaaagctactaaatatttccctttaattaaaggcataaaaaacaattatcctaattttgctttagaacatttctttgctaccgcaaattatttgtggactttatgggaagctggaattttgtatttaaggaagaatcaaacaactttgacttttaaaggtaaaccatattcttgggaacacagacagctagtgcaacataatgggcaacaacataaaagtcaccttcaatccagacaaaatagcagcatggtggcctgcagtgggcacttattacacaaccacttatcctcagaatcagtcagtgtttcaaccaggaatttatcaaacaacatctctgataaatcccaaaaatcaacaagaactggactctgttcttataaacagatacaaacagatagactggaacacttggcaaggatttcctgtggatcaaaaattaccattggtcagcagggatcctcccccaaaaccttatataaatcaatcagctcaaactttcgaaatcaaacctgggcctataatagttcccgg

SEQ ID NO: 5 - protein, 65 aa, Artificial Sequence
  Synthesized Construct
GILEXXXXXXXXXXXXXXXXXXXXXXXXXXXXXXXXXXXXXXXXXXXXXXXXXXXXXXXXXXXXL

SEQ ID NO: 6 - protein, 4 aa, Artificial Sequence
  Synthesized Construct
WLWG

SEQ ID NO: 7 - protein, 195 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIAAGRSPSQSPSQSSANC

SEQ ID NO: 8 - protein, 217 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 9 - protein, 230 aa, Artificial Sequence
  Synthesized Construct
MGTYMLTNSELLSLINDMPITNDQKKLMSNNVQIVRAGWLWGMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 10 - protein, 229 aa, Artificial Sequence
  Synthesized Construct
MGTYMLTNSELLSLINDMPITNDQKKLMSNNVQIVRWLWGAMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 11 - protein, 208 aa, Artificial Sequence
  Synthesized Construct
MGTYMLTNSELLSLINDMPITNDQKKLMSNNVQIVRWLWGAMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIAAGRSPSQSPSQSRESQC

SEQ ID NO: 12 - protein, 246 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILGGGGSGGGGETYMLTNSELLSLINDMPITNDQKKLMSNNVQIVREGGGGSGGGGLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 13 - protein, 191 aa, Artificial Sequence
  Synthesized Construct
MDIDPYKEFGSSYQLLNFLPLDFFPDLNALVDTATALYEEELTGREHCSPHHTAIRQALVCWDELSELLSLINDMPITNDQKKLMSIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 14 - protein, 232 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILEVNAGVTTPVSTYMLTNSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 15 - protein, 253 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRGTPSPRRRRSQSPRRRRSQSPSANCDISNIETVIEFQQKNNRLLEITREFSVNAGVTTPVSTYMLTNSELLSLINDMPITNDQKKLMSNN

SEQ ID NO: 16 - protein, 253 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIAAAGGAAASASPAAATPSPAAARSQSPAAAASQSPSANCDISNIETVIEFQQKNNRLLEITREFSVNAGVTTPVSTYMLTNSELLSLINDMPITNDQKKLMSNN

SEQ ID NO: 17 - protein, 258 aa, Artificial Sequence
  Synthesized Construct
MVSNIETVIEFQQKNNRLLEITREFSVNAGVTTPVSTYMLTNSELLSLINDMPITNDQKKLMSNNWLWGAMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 18 - protein, 237 aa, Artificial Sequence
  Synthesized Construct
MVSNIETVIEFQQKNNRLLEITREFSVNAGVTTPVSTYMLTNSELLSLINDMPITNDQKKLMSNNWLWGAMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIAAGRSPSQSPSQSRESQC

SEQ ID NO: 19 - protein, 238 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILEVNAGVTTPVSTYMLTNSELLSLINDMPITNDQKKLMSNNVQIVRELEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 20 - protein, 239 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVGILEVNAGVTTPVSTYMLTNSELLSLINDMPITNDQKKLMSNNVQIVRELERTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 21 - protein, 253 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIAAAGGAAASASPAAATPSPAAARSQSPRRRRSQSPSANCDISNIETVIEFQQKNNRLLEITREFSVNAGVTTPVSTYMLTNSELLSLINDMPITNDQKKLMSNN

SEQ ID NO: 22 - protein, 206 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILETYMLTNSELLSLINDMPITNDQKKLMSNNVQIVRELEQVRTIIVNHVNDTWGLKVRQSLWFHLSCLTFGQHTVQEFLVSFGVWIRTPAPYRPPNAPILSTLPEHTVIAAGRSPSQSPSQSSANC

SEQ ID NO: 23 - protein, 229 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVGILETYMLTNSELLSLINDMPITNDQKKLMSNNVQIVRELERTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 24 - protein, 228 aa, Artificial Sequence
  Synthesized Construct
MDIDPYKEFGSSYQLLNFLPADFFPAAAVLADTATALYEEELTGREHCSPHHTAIRQALVCWDELTKLIAWMSSNITSGILETYMLTNSELLSLINDMPITNDQKKLMSNNVQIVRELEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 25 - protein, 228 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILETYMLTNSELLSLINDMPITNDQKKLMSNNVQIVRELEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 26 - protein, 219 aa, Artificial Sequence
  Synthesized Construct
MGNSELLSLINDMPITNDQKKLMSNNWLWGAMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 27 - protein, 198 aa, Artificial Sequence
  Synthesized Construct
MGNSELLSLINDMPITNDQKKLMSNNWLWGAMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIAAGRSPSQSPSQSRESQC

SEQ ID NO: 28 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNSELLSLINDMPITNDQKKLMSNNASSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 29 - protein, 218 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVGILENSELLSLINDMPITNDQKKLMSNNLERTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 30 - protein, 217 aa, Artificial Sequence
  Synthesized Construct
MDIDPYKEFGSSYQLLNFLPADFFPAAAVLADTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 31 - protein, 238 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVGILGGGGSGGGGETYMLTNSELLSLINDMPITNDQKKLMSNNVQIVRELERTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 32 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNIISGILETYMLTNSELLSLINDMPITNDQKKLMSNNVQIVREGGGGSGGGGLEQVRTIIVNHVNDTWGLKVRQSLWFHLSCLTFGQHTVQEFLVSFGVWIRTPAPYRPPNAPILSTLPEHTVIAAGRSPSQSPSQSSANC

SEQ ID NO: 33 - protein, 237 aa, Artificial Sequence
  Synthesized Construct
MDIDPYKEFGSSYQLLNFLPADFFPAAAVLADTATALYEEELTGREHCSPHHTAIRQALVCWDELTKLIAWMSSNITSGILETYMLTNSELLSLINDMPITNDQKKLMSNNVQIVREGGGGSGGGGLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 34 - protein, 237 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILGGGGSGGGGETYMLTNSELLSLINDMPITNDQKKLMSNNVQIVRELEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 35 - protein, 212 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSNSELLSLINDMPITNDQKKLMSNNEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 36 - protein, 209 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSELLSLINDMPITNDQKKLMSNEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 37 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNSELLSLINDMPITNDQKKLMSNNASSAAAAAAAAAIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 38 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNSELLSLINDMPITNDQKKLMSNNASSELELELELEIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 39 - protein, 237 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILETYMLTNSELLSLINDMPITNDQKKLMSNNVQIVREGGGGSGGGGLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 40 - protein, 216 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLINDLPASNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 41 - protein, 216 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLINDAPAANDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 42 - protein, 216 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLINDAAAANDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 43 - protein, 218 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNIGILENSELLSLINDMPITNDQKKLMSNNLETSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 44 - protein, 218 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSEQVRGILENSELLSLINDMPITNDQKKLMSNNLETIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 45 - protein, 217 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILANSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 46 - protein, 216 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 47 - protein, 220 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILEENSELLSLINDMPITNDQKKLMSNNEELEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 48 - protein, 217 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILEIKQSLMTDSLSNPNNLNNDIKLEMLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 49 - protein, 217 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIAAAGGAAASASPAAATPSPAAARSQSPAAAASQSPSANC

SEQ ID NO: 50 - protein, 217 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIAAAGGAAASASPAAATPSPAAARSQSPRRRRSQSPSANC

SEQ ID NO: 51 - protein, 212 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIDIDYINKLQNTITSDWTPCTVSRRRRSQSPRRRR

SEQ ID NO: 52 - protein, 204 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIDIDYINKLQNTITSDWTPCTVSRRRR

SEQ ID NO: 53 - protein, 191 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRGGARASQSANC

SEQ ID NO: 54 - protein, 217 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPPPPILSTLPEHTVIRRRGGARASRSPRRRTPSPRRRRSQSPRRRRSQSPSANC

SEQ ID NO: 55 - protein, 220 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILPENSELLSLINDMPITNDQKKLMSNNPELEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 56 - protein, 216 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLIHDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 57 - protein, 216 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLINDMPAANDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 58 - protein, 216 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLINDAPASNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 59 - protein, 217 aa, Artificial Sequence
  Synthesized Construct
MDIDPYKEFGSSYQLLNFLPLDFFPDLNALVDTATALYEEELTGREHCSPHHTAIRQALVSWDELTKLIAWMSSNITSGILENSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 60 - protein, 213 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSTNSELLSLINDMPITNDQKKLMSNNEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 61 - protein, 214 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSLTNSELLSLINDMPITNDQKKLMSNNEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 62 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSMLTNSELLSLINDMPITNDQKKLMSNNEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 63 - protein, 213 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSNSELLSLINDMPITNDQKKLMSNNVEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 64 - protein, 214 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSNSELLSLINDMPITNDQKKLMSNNVQEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 65 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSNSELLSLINDMPITNDQKKLMSNNVQIEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 66 - protein, 207 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNSELLSLINDMPITNDQKKLMSNNQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 67 - protein, 208 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSTNSELLSLINDMPITNDQKKLMSNNQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 68 - protein, 208 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNSELLSLINDMPITNDQKKLMSNNVQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 69 - protein, 209 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSTNSELLSLINDMPITNDQKKLMSNNVQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 70 - protein, 188 aa, Artificial Sequence
  Synthesized Construct
MDIDPYKEFGSSYQLLNFLPLDFFPDLNALVDTATALYEEELTGREHCSPHHTAIRQALVCNSELLSLINDMPITNDQKKLMSNNVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 71 - protein, 188 aa, Artificial Sequence
  Synthesized Construct
MDIDPYKEFGSSYQLLNFLPLDFFPDLNALVDTATALYEEELTGREHCSPHHTAIRQALVCWNSELLSLINDMPITNDQKKLMSNNNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 72 - protein, 188 aa, Artificial Sequence
  Synthesized Construct
MDIDPYKEFGSSYQLLNFLPLDFFPDLNALVDTATALYEEELTGREHCSPHHTAIRQALVCWDNSELLSLINDMPITNDQKKLMSNNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 73 - protein, 188 aa, Artificial Sequence; identical to SEQ ID NO: 72
  Synthesized Construct

SEQ ID NO: 74 - protein, 186 aa, Artificial Sequence
  Synthesized Construct
MDIDPYKEFGSSYQLLNFLPLDFFPDLNALVDTATALYEEELTGREHCSPHHTAIRQALVCWDELLSLINDMPITNDQKKLMSNNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 75 - protein, 188 aa, Artificial Sequence
  Synthesized Construct
MDIDPYKEFGSSYQLLNFLPLDFFPDLNALVDTATALYEEELTGREHCSPHHTAIRQALVSNSELLSLINDMPITNDQKKLMSNNVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 76 - protein, 188 aa, Artificial Sequence
  Synthesized Construct
MDIDPYKEFGSSYQLLNFLPLDFFPDLNALVDTATALYEEELTGREHCSPHHTAIRQALVSWNSELLSLINDMPITNDQKKLMSNNNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 77 - protein, 188 aa, Artificial Sequence
  Synthesized Construct
MDIDPYKEFGSSYQLLNFLPLDFFPDLNALVDTATALYEEELTGREHCSPHHTAIRQALVSWDNSELLSLINDMPITNDQKKLMSNNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 78 - protein, 188 aa, Artificial Sequence
  Synthesized Construct
MDIDPYKEFGSSYQLLNFLPLDFFPDLNALVDTATALYEEELTGREHCSPHHTAIRQALVSWDENSELLSLINDMPITNDQKKLMSNNVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 79 - protein, 186 aa, Artificial Sequence
  Synthesized Construct
MDIDPYKEFGSSYQLLNFLPLDFFPDLNALVDTATALYEEELTGREHCSPHHTAIRQALVSWDELLSLINDMPITNDQKKLMSNNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 80 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGINSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 81 - protein, 214 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGNSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 82 - protein, 214 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSINSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 83 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSILNSELLSLINDMPITNDQKKLMSNNLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 84 - protein, 215 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLINDMPITNDQKKLMSNNEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 85 - protein, 216 aa, Artificial Sequence
  Synthesized Construct
MDIDPYKEFGSSYQLLNFLPLDFFPDLNALVDTATALYEEELTGREHCSPHHTAIRQALVCWDELTKLIAWMSSNITSGILNSELLSLINDMPITNDQKKLMSNNVEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 86 - protein, 24 aa, Artificial Sequence
  Synthesized Construct
NSELLSLINDMPITNDQKKLMSSN

SEQ ID NO: 87 - protein, 24 aa, Artificial Sequence
  Synthesized Construct
NSELLSLINDMPITNDQKKLMFNN

SEQ ID NO: 88 - protein, 29 aa, Artificial Sequence
  Synthesized Construct
GILENSELLSLINDMPITNDQKKLMSNNL

SEQ ID NO: 89 - protein, 26 aa, Artificial Sequence
  Synthesized Construct
ANSELLSLINDMPITNDQKKLMSNNL

SEQ ID NO: 90 - protein, 25 aa, Artificial Sequence
  Synthesized Construct
NSELLSLINDMPITNDQKKLMSNNL

SEQ ID NO: 91 - protein, 32 aa, Artificial Sequence
  Synthesized Construct
GILEENSELLSLINDMPITNDQKKLMSNNEEL

SEQ ID NO: 92 - protein, 29 aa, Artificial Sequence
  Synthesized Construct
GILEIKQSLMTDSLSNPNNLNNDIKLEML

SEQ ID NO: 93 - protein, 21 aa, Artificial Sequence
  Synthesized Construct
SELLSLINDMPITNDQKKLMS

SEQ ID NO: 94 - protein, 38 aa, Artificial Sequence
  Synthesized Construct
GILTYMLTNSELLSLINDMPITNDQKKLMSNNVQIVRL

SEQ ID NO: 95 - protein, 43 aa, Artificial Sequence
  Synthesized Construct
GILVNAGVTTPVSTYMLTNSELLSLINDMPITNDQKKLMSNNL

SEQ ID NO: 96 - protein, 48 aa, Artificial Sequence
  Synthesized Construct
GILVNAGVTTPVSTYMLTNSELLSLINDMPITNDQKKLMSNNVQIVRL

SEQ ID NO: 97 - protein, 67 aa, Artificial Sequence
  Synthesized Construct
GILSNIETVIEFQQKNNRLLEITREFSVNAGVTTPVSTYMLTNSELLSLINDMPITNDQKKLMSNNL

SEQ ID NO: 98 - protein, 21 aa, Artificial Sequence
  Synthesized Construct
ELLSLINDMPITNDQKKLMSN

SEQ ID NO: 99 - protein, 28 aa, Artificial Sequence
  Synthesized Construct
GILNSELLSLINDLPASNDQKKLMSNNL

SEQ ID NO: 100 - protein, 28 aa, Artificial Sequence
  Synthesized Construct
GILNSELLSLINDAPAANDQKKLMSNNL

SEQ ID NO: 101 - protein, 28 aa, Artificial Sequence
  Synthesized Construct
GILNSELLSLINDAAAANDQKKLMSNNL

SEQ ID NO: 102 - protein, 32 aa, Artificial Sequence
  Synthesized Construct
GILPENSELLSLINDMPITNDQKKLMSNNPEL

SEQ ID NO: 103 - protein, 29 aa, Artificial Sequence
  Synthesized Construct
GILENSELLSLIHDMPITNDQKKLMSNNL

SEQ ID NO: 104 - protein, 28 aa, Artificial Sequence
  Synthesized Construct
GILNSELLSLINDMPAANDQKKLMSNNL

SEQ ID NO: 105 - protein, 28 aa, Artificial Sequence
  Synthesized Construct
GILNSELLSLINDAPASNDQKKLMSNNL

SEQ ID NO: 106 - protein, 25 aa, Artificial Sequence
  Synthesized Construct
TNSELLSLINDMPITNDQKKLMSNN

SEQ ID NO: 107 - protein, 26 aa, Artificial Sequence
  Synthesized Construct
LTNSELLSLINDMPITNDQKKLMSNN

SEQ ID NO: 108 - protein, 27 aa, Artificial Sequence
  Synthesized Construct
MLTNSELLSLINDMPITNDQKKLMSNN

SEQ ID NO: 109 - protein, 25 aa, Artificial Sequence
  Synthesized Construct
NSELLSLINDMPITNDQKKLMSNNV

SEQ ID NO: 110 - protein, 26 aa, Artificial Sequence
  Synthesized Construct
NSELLSLINDMPITNDQKKLMSNNVQ

SEQ ID NO: 111 - protein, 26 aa, Artificial Sequence
  Synthesized Construct
TNSELLSLINDMPITNDQKKLMSNNV

SEQ ID NO: 112 - protein, 24 aa, Artificial Sequence
  Synthesized Construct
NSELLSLINDMPITNDQKKLMLNN

SEQ ID NO: 113 - protein, 24 aa, Artificial Sequence
  Synthesized Construct
NSELLSLINDMPITNDQKELMFNN

SEQ ID NO: 114 - protein, 24 aa, Artificial Sequence
  Synthesized Construct
NSELLSLINDMPITNDQKQLMSNN

SEQ ID NO: 115 - protein, 27 aa, Artificial Sequence
  Synthesized Construct
NSELLSLINDMPITNDQKKLMSNNVQI

(19) United States
(12) Patent Application Publication
(10) Pub. No.: US 2016/0039883 A1
(43) Pub. Date: Feb. 11, 2016

(54) PALIVIZUMAB EPITOPE-BASED VIRUS-LIKE PARTICLES

(71) Applicants: VLP BIOTECH, INC., San Diego, CA (US); MEDIMMUNE, LLC, Gaithersburg, MD (US)

(72) Inventors: David R. MILICH, Escondido, CA (US); David C. WHITACRE, San Diego, CA (US); Jeanne H. SCHICKLI, Frisco, CO (US)

(73) Assignees: MEDIMMUNE, LLC, Gaithersburg, MD (US); VLP BIOTECH, INC., San Diego, CA (US)

(21) Appl. No.: 14/774,115

(22) PCT Filed: Mar. 14, 2014
(86) PCT No.: PCT/US14/29297

§ 371 Date: Sep. 9, 2015

Related U.S. Application Data
(60) Provisional application No. 61/802,240, filed on Mar. 15, 2013.

(51) Int. Cl. C07K 14/005; G01N 33/68; C12N 7/00; A61K 39/29; A61K 39/155

(52) U.S. Cl. CPC C07K 14/005; A61K 39/29; A61K 39/155; C12N 7/00; G01N 33/6854; C07K 2319/00; A61K 2039/5258

(57) ABSTRACT

The present disclosure generally relates to immunogens for eliciting an antibody response against respiratory syncytial virus (RSV). More specifically, the present disclosure relates to virus-like particles (VLPs) including a RSV F protein epitope, as well as methods of use thereof. Respiratory syncytial virus (RSV) is a major cause of lower respiratory tract disease in infants and young children (Hall et al., NEJM, 360:5888-598, 2009; and Nair et al., Lancet, 375:1545-1555, 2010) and a vaccine to protect this young population is of high priority.

### CROSS-REFERENCE TO RELATED APPLICATIONS

This application claims benefit of U.S. Provisional Application No. 61/802,240, filed Mar. 15, 2013, which is hereby incorporated by reference in its entirety.

### FIELD

The present disclosure generally relates to immunogens for eliciting an antibody response against respiratory syncytial virus (RSV). More specifically, the present disclosure relates to virus-like particles (VLPs) including a RSV F protein epitope, as well as methods of use thereof.

### BACKGROUND

Respiratory syncytial virus (RSV) is a major cause of lower respiratory tract disease in infants and young children (Hall et al., NEJM, 360:5888-598, 2009; and Nair et al., Lancet, 375:1545-1555, 2010) and a vaccine to protect this young population is of high priority. Development of a RSV vaccine has been hampered by the incidence of enhanced respiratory disease (ERD) following vaccination with formalin-inactivated whole virus vaccine (FI-RSV) (Fulginiti et al., Am J Epidemiol, 89:435-448, 1969; Kapikian et al., Am J Epidemiol, 89:405-421, 1969; and Kim et al., Am J Epidemiol, 89:422-434, 1969). Specifically, FI-RSV administered to infants and children did not protect against RSV infection and actually increased the risk of severe respiratory disease following RSV infection during the subsequent RSV season. Vaccine-induced ERD has been duplicated in animal models of RSV infection leading to a generally accepted view that a skewed Th2 T cell response and the production of non-functional anti-RSV antibodies (i.e., low avidity, non-neutralizing, non-fusion inhibiting and non-protective) are important contributing factors to the development of ERD and should be avoided in the development of RSV vaccine candidates.

A number of RSV vaccine candidates have subsequently been developed including: live attenuated vaccines with cold-passaged (cp), temperature-sensitive (ts) mutations; recombinant virus with deletion mutations (SH, NSI or NS2); and combinations thereof. In general these live attenuated vaccines exhibited residual virulence, genetic instability, and/or insufficient immunogenicity in clinical testing (Schickli et al., Human Vaccines, 5:582-591, 2009; Wright et al., J Infect Dis, 182:1331-1342, 2000; and Karron et al., J Infect Dis, 191:1093-1104, 2005). Subunit vaccines including purified F glycoprotein (Groothuis et al., J Infect Dis, 177:467-469, 1998), recombinant chimeric F/G glycoproteins (Prince et al., J Virol, 77:13256-13160, 2003), plasmid DNA encoding F and G glycoproteins (Bembridge et al., J Gen Virol, 81:2519-2523, 2000; and Li et al., Virology, 269:54-65, 2000) and G protein peptides (De Waal et al., Vaccine, 22:915-922, 2004) have also been developed. However, no non-replicating RSV vaccine candidates have been tested in immunologically naïve infants and will require a compelling safety profile in animal models due to the failed FI-RSV trial. Furthermore, immunization with both the F (Murphy et al., Vaccine, 8:497-502, 1990) and G (Hancock et al., J Virol, 70:7783-7791, 1996; and Johnson et al., J Virol, 72:2871-2880, 1998) glycoproteins of RSV have been reported to induce ERD.

Thus the art needs immunogens with a better safety profile for eliciting RSV neutralizing antibodies. In particular RSV immunogens with reduced risk for ERD induction are desirable.

### SUMMARY

The present disclosure generally relates to immunogens for eliciting an antibody response against respiratory syncytial virus (RSV). More specifically, the present disclosure relates to virus-like particles (VLPs) including a RSV F protein epitope, as well as methods of use thereof.

The present disclosure provides antigenic compositions comprising a hybrid woodchuck hepadnavirus core antigen, wherein the hybrid core antigen is a fusion protein comprising a respiratory syncytial virus (RSV) F polypeptide and a woodchuck hepadnavirus core antigen, and wherein the fusion protein is capable of assembling as a hybrid virus-like particle (VLP). In some embodiments, the RSV F polypeptide comprises a palivizumab epitope (e.g., capable of being bound by palivizumab). In some embodiments, the amino acid sequence of the RSV F polypeptide comprises SEQ ID NO:3, one of SEQ ID NOS:86-111, or is at least 95% identical to one of SEQ ID NOS:86-111. In additional embodiments, the RSV F polypeptide may be from 20 to 60 amino acids in length, or any integer between 20 to 30, 40, or 50 amino acids in length. In some embodiments, the RSV F polypeptide is inserted at a position within the woodchuck hepadnavirus core antigen selected from the group consisting of N-terminal, 44, 71, 72, 73, 74, 75, 76, 77, 78, 81, 82, 83, 84, 85, 92, 149 and C-terminal as numbered according to SEQ ID NO:1. In other embodiments, the RSV F polypeptide is inserted at a position within the woodchuck hepadnavirus core antigen selected from the group consisting of N-terminal, 74, 78, 81, 82, 149 and C-terminal. In some embodiments, the amino acid sequence of the hybrid core antigen comprises one of SEQ ID NOS:7-85, or is at least 95% identical to one of SEQ ID NOS:7-85. In some embodiments, the hybrid VLP binds to palivizumab. In some embodiments, the hybrid VLP binds to palivizumab and/or is selected from the group consisting of VLP018, VLP019, VLP023, VLP027, VLP033, VLP045, VLP046, VLP048, VLP049, VLP050, VLP052, VLP053, VLP059, VLP060, VLP061, VLP062, VLP063, VLP064, VLP068, VLP072, VLP074, VLP075, VLP076, VLP078, VLP080, VLP087, VLP088, VLP089, VLP090, VLP091, VLP092, VLP093, VLP094, VLP095, VLP096, VLP097, VLP098, VLP099, VLP111, VLP112, VLP113, VLP120, VLP123, VLP124, VLP125, VLP128, VLP129, VLP130, VLP131, VLP132, VLP133, VLP134, and VLP135. In some embodiments, the hybrid VLP elicits a high titer, anti-RSV F protein IgG response. In additional embodiments, the hybrid VLP elicits a high titer, anti-RSV F protein IgG response and/or is selected from the group consisting of VLP018, VLP019, VLP023, VLP027, VLP033, VLP045, VLP046, VLP048, VLP049, VLP050, VLP052, VLP053, VLP059, VLP060, VLP061, VLP062, VLP063, VLP064, VLP068, VLP072, VLP073, VLP074, VLP075, VLP076, VLP078, VLP080, VLP087, VLP088, VLP089, VLP090, VLP091, VLP092, VLP093, VLP094, VLP095, VLP096, VLP097, VLP098, VLP099, VLP111, VLP112, VLP113, VLP120, VLP123, VLP124, VLP125, VLP128, VLP129, VLP130, VLP131, VLP132, VLP133, VLP134, and VLP135. In some embodiments, the hybrid VLP elicits one or both of a measurable neutralizing antibody response against RSV subtype A and protective immune response against RSV subtype A. In additional embodiments, the hybrid VLP elicits one or both of a measurable neutralizing antibody response against RSV subtype A and protective immune response against RSV subtype A and/or is selected from the group consisting of VLP018, VLP019, VLP049, VLP050, VLP052, VLP059, VLP060, VLP062, VLP074, VLP075, VLP078, VLP080, VLP087, VLP088, VLP090, VLP091, VLP093, VLP096, VLP097, VLP098, VLP113, VLP123, VLP128, VLP130, VLP131, VLP132, VLP133, VLP134, and VLP135. In some embodiments, the hybrid VLP elicits an intermediate to high titer neutralizing antibody response against RSV subtype A. In additional embodiments, the hybrid VLP elicits an intermediate to high titer neutralizing antibody response against RSV subtype A and/or is selected from the group consisting of VLP018, VLP019, VLP049, VLP059, VLP060, VLP074, VLP075, VLP078, VLP080, VLP087, VLP088, VLP093, VLP097, VLP123, VLP128, VLP130, VLP131, VLP132, and VLP135. In some embodiments, the hybrid VLP elicits an intermediate to high level of protection from RSV subtype A infection. In additional embodiments, the hybrid VLP elicits an intermediate to high level of protection from RSV subtype A infection and/or is selected from the group consisting of VLP018, VLP019, VLP049, VLP050, VLP059, VLP060, VLP062, VLP074, VLP075, VLP078, VLP080, VLP087, VLP088, VLP090, VLP093, and VLP096. In some embodiments, the hybrid VLP is selected from the group consisting of VLP019, VLP049, VLP075, VLP080, VLP087, VLP090, VLP093, VLP097, VLP123, VLP128, VLP131, VLP132, AND VLP135. In some embodiments, the hybrid VLP comprises a combination of two, three, four, or five different hybrid VLPs. In some embodiments, the hybrid VLP comprises two, three, four, or five different fusion proteins capable of assembling as a single hybrid VLP. In some embodiments, the hybrid VLP comprises two, three, four, or five different fusion proteins capable of assembling as a single hybrid VLP. In some embodiments, the hybrid VLP comprises a combination of from two to all of the group consisting of VLP018, VLP019, VLP023, VLP027, VLP033, VLP045, VLP046, VLP048, VLP049, VLP050, VLP052, VLP053, VLP059, VLP060, VLP061, VLP062, VLP063, VLP064, VLP068, VLP072, VLP074, VLP075, VLP076, VLP078, VLP080, VLP087, VLP088, VLP089, VLP090, VLP091, VLP092, VLP093, VLP094, VLP095, VLP096, VLP097, VLP098, VLP099, VLP111, VLP112, VLP113, VLP120, VLP123, VLP124, VLP125, VLP128, VLP129, VLP130, VLP131, VLP132, VLP133, VLP134, and VLP135. In some embodiments, the fusion protein comprises one, two or three copies of the RSV F polypeptide. In additional embodiments, each copy of the RSV F polypeptide is inserted at a different position within the woodchuck hepadnavirus core antigen. In further embodiments, the two or the three copies of the RSV F polypeptide are inserted in tandem in a single position within the woodchuck hepadnavirus core antigen. In some embodiments, the present disclosure also provides a vaccine comprising the antigenic composition of the present disclosure, and an adjuvant.

In additional embodiments, the present disclosure provides a method for eliciting an immune response, comprising administering to a mammal an effective amount of the antigenic composition of the present disclosure. In brief, the antigenic composition comprises a hybrid woodchuck hepadnavirus core antigen, wherein the hybrid core antigen is a fusion protein comprising a respiratory syncytial virus (RSV) F polypeptide and a woodchuck hepadnavirus core antigen, and wherein the fusion protein is capable of assembling as a hybrid virus-like particle (VLP). In some embodiments, the RSV F polypeptide comprises a palivizumab epitope (e.g., capable of being bound by palivizumab). Various hybrid core antigens for use with the methods are described in detail in the preceding paragraph of the summary. In some embodiments, the immune response comprises a RSV-reactive antibody response. In some embodiments, the present disclosure provides a method for reducing RSV infection or preventing RSV disease in a mammal in need thereof, comprising administering to the mammal an effective amount of the antigenic composition (e.g., vaccine) of the present disclosure according to a suitable vaccine regimen comprising an initial immunization and one or more subsequent immunizations. In some embodiments, the mammal is a human. In some embodiments, the human is a baby (for early childhood immunization methods). In some embodiments the human is a pregnant female (for maternal immunization methods). In some embodiments the present disclosure provides a method for protecting a baby against RSV infection or RSV disease, comprising administering an effective amount of the antigenic composition to a pregnant female carrying a baby so as to increase RSV-specific antibodies of the pregnant female, wherein a portion of the RSV-specific antibodies are transferred via the female's placenta to the baby during gestation, and/or transferred via breast milk to the baby after birth, thereby protecting the baby against RSV infection or RSV disease. In some embodiments, the baby is a fetus (e.g., unborn baby), a neonate (e.g., newborn less than one month old), or an infant (e.g., one to 12 months old). In some embodiments, the RSV-specific antibodies are detectable in serum of the baby at or following birth. In some embodiments, the RSV-specific antibodies comprise IgG antibodies. In some embodiments, the IgG antibodies are RSV-neutralizing antibodies. In some embodiments, protecting the baby against RSV infection comprises reducing RSV titers in nasal secretions of the baby after exposure to RSV as compared to that of an RSV-infected baby. In some embodiments, protecting the baby against RSV disease comprises reducing incidence or severity of a lower respiratory tract infection with RSV as compared to a baby with RSV-induced bronchiolitis. In some aspects, the subsequent immunization is in one boost. In other aspects, the subsequent immunization is in two boosts.

In additional embodiments, the present disclosure provides a method for screening anti-RSV antibodies comprising: a) measuring binding of an antibody or fragment thereof to a hybrid woodchuck hepadnavirus core antigen, wherein the hybrid core antigen is a fusion protein comprising a respiratory syncytial virus (RSV) F polypeptide and a woodchuck hepadnavirus core antigen, and wherein said fusion protein assembles as a hybrid virus-like particle (VLP); and b) measuring binding of the antibody or fragment thereof to a woodchuck hepadnavirus VLP devoid of the RSV F polypeptide; and c) determining that the antibody or fragment thereof is specific for the RSV F polypeptide when the antibody or fragment thereof binds to the hybrid VLP but not the woodchuck hepadnavirus VLP devoid of the RSV F polypeptide. In some embodiments, the RSV F polypeptide comprises a palivizumab epitope (e.g., capable of being bound by palivizumab). Various hybrid core antigens for use with the methods are described in detail in the preceding paragraphs of the summary.

Moreover, the present disclosure provides a polynucleotide encoding a hybrid woodchuck hepadnavirus core antigen, wherein the hybrid core antigen is a fusion protein comprising a respiratory syncytial virus (RSV) F polypeptide and a woodchuck hepadnavirus core antigen. In some embodiments, the RSV F polypeptide comprises a palivizumab epitope (e.g., capable of being bound by palivizumab). Various hybrid core antigens are described in detail in the preceding paragraphs of the summary. In additional embodiments, the present disclosure provides an expression construct comprising a polynucleotide described herein, in operable combination with a promoter. In additional embodiments, the present disclosure also provides an expression vector comprising the expression construct described herein. In additional embodiments, the present disclosure provides a host cell comprising an expression vector described herein.

### BRIEF DESCRIPTION OF THE DRAWINGS

FIG. 1 provides a schematic of the woodchuck hepadnavirus core antigen (WHcAg) structure illustrating positional tolerance for epitope insertions. Circles indicate insert positions that are tolerant for particle assembly including positions: 1 (N-terminus), 44, 71, 72, 73, 74, 75, 76, 77, 78, 81, 82, 83, 84, 85, 92, and 187 (C-terminus). The C-terminus of WHcAg truncated at residue 149 (e.g., devoid of residues 150-188) is also tolerant for particle assembly. In contrast, squares indicate insert positions that are intolerant for particle assembly including positions: 21, 66, 79, 80, 86 and 91. Position numbering is based on the full length WHcAg amino acid sequence set forth as SEQ ID NO:1. The WHcAg truncated at position 149 is set forth as SEQ ID NO:2. The RSV F²⁵⁴⁻²⁷⁷ epitope (SEQ ID NO:3) is inserted at WHcAg position 78 in this illustration.

FIG. 2 provides a flow chart of hybrid (WHcAg-RSV) virus-like particle (VLP) testing.

FIG. 3A shows the antigenicity of hybrid, WHcAg-RSV VLPs as solid phase antigens for binding to palivizumab. FIG. 3B shows the antigenicity of hybrid VLPs in solution as inhibitors of palivizumab binding to RSV F protein.

FIG. 4A through FIG. 4D show the antigenicity of hybrid, WHcAg-RSV VLPs as solid phase antigens for binding to four RSV-reactive monoclonal antibodies.

FIG. 5 shows that hybrid, WHcAg-RSV VLPs are capable of inhibiting palivizumab neutralization of RSV.

FIG. 6A shows the immunogenicity of hybrid, WHcAg-RSV VLPs. FIG. 6B shows antibodies to hybrid, WHcAg-RSV VLPs are capable of inhibiting palivizumab-binding to solid-phase RSV recombinant F (rF) protein.

FIG. 7A and FIG. 7B show the immunogenicity of hybrid, WHcAg-RSV VLPs. FIG. 7C and FIG. 7D show the protective efficacy of hybrid, WHcAg-RSV VLPs.

FIG. 8A through FIG. 8F shows the neutralization of RSV-A, RSV-B and a palivizumab escape mutant (MARM S275F) by VLP019 antiserum. Dilutions of heat-inactivated serum or palivizumab were mixed with 100-200 PFU RSV and incubated for 1 hour, before titers were measured by plaque assay. Anti-VLP-19 serum neutralized wtRSV A2 (FIG. 8A), several recent RSV A clinical isolates (FIG. 8B), two RSV B clinical isolates (FIG. 8C and FIG. 8D) and several antibody-escape mutants (FIG. 8E and FIG. 8F).

FIG. 9 provides a comparison of the protective efficacy of hybrid, WHcAg-RSV VLPs in alum and incomplete Freund's adjuvant (IFA) in mice.

FIG. 10 provides electron micrographs of VLPs. Cryoelecton microscopy analysis was performed on WHcAg, VLP-19 and VLP-19 incubated with palivizumab Fabs in PBS. The samples were vitrified in liquid ethane and imaged with an FEI Tecnai T12 electron microscope at NanoImaging Services, Inc.

FIG. 11A through FIG. 11D illustrates results of in vitro analyses of VLP-19 compared to WHcAg and sF. In FIG. 11A, VLPs were separated by SDS-PAGE: VLP-19 (lanes 1, 3 and 5); and WHcAg (2, 4 and 6). Total protein was visualized by Sypro Ruby stain (lanes 1 and 2). Western blots were probed with anti-WHc Ab (lanes 3 and 4) or anti-RSV F palivizumab (lanes 5 and 6). In FIG. 11B, ELISA plates were coated with VLP-19, sF or WHcAg before being incubated with dilutions of palivizumab. In FIG. 11C, ELISA plates were coated with sF before being incubated with dilutions of competitor (VLP-19, sF or WHcAg) mixed with 100 ng/ml palivizumab. In FIG. 11D, ELISA plates were coated with VLP-19, sF or WHcAg before being incubated with dilutions of human plasma. For all ELISA assays, bound IgG was detected by HRP-conjugated anti-human IgG Ab, followed by incubation with tetramethylbenzidine. The reaction was stopped with 0.1N HCl and optical density (OD) was read at 450 nm. Each ELISA assay was performed in triplicate, and data shown represents a single assay.

FIG. 12A through FIG. 12C shows that immunization with VLP-19 protects mice from challenge and elicits neutralizing Ab and F-specific IgG. BALB/c Mice (n=5) were intramuscularly dosed with 100 μL of either 40 μg VLP-19 formulated in incomplete Freund's adjuvant (IFA) or PBS alone on days 0 and 14, or were infected with 10⁶ PFU wtRSV A2 on day 0. On day 28, sera were sampled and mice were challenged with 10⁶ PFU wtRSV A2. On day 32 lungs were harvested. FIG. 12A shows the titer of RSV in lung tissue as determined by plaque assay. FIG. 12B shows the RSV microneutralization titers of heat-inactivated sera. FIG. 12C shows the sF-specific IgG titer Sera as determined by ELISA. Data points for each animal are shown with a line through the mean. T-test was performed to determine p value and “ns” indicates “not significant”. The data shown were from immunization with IFA. Immunization using a proprietary adjuvant yielded similar results.

FIG. 13A and FIG. 13B illustrate that murine anti-VLP-19 sera neutralizes RSV and competes with palivizumab for binding to sF. FIG. 13A shows results of a RSV PRNT neutralization assay performed with heat-inactivated sera from VLP-19 immunized mice as compared to palivizumab. FIG. 13B shows that sera from VLP-19 immunized mice competes with palivizumab for binding to sF. ELISA plates were coated with sF and incubated with a mixture of a constant amount of palivizumab mixed with dilutions of either negative control sera or anti-VLP-19 sera. Bound palivizumab was detected with HRP-conjugated anti-human Ab. Following washes, color was developed with tetramethylbenzidine followed by 0.1N HCl. Optical density was read at 450 nm and percent inhibition was calculated by comparison to a negative control. Data shown is representative of three independent experiments.

FIG. 14A and FIG. 14B show the immunogenicity and efficacy of VLP-19 administered in the absence of adjuvant. Groups of three B10xB10.S F1 mice were immunized intraperitoneally with the indicated doses of VLP-19 in PBS at weeks 0 and 6. Mice were bled, pooled serum were tested. FIG. 14A shows the anti-RSV F protein IgG titers of anti-VLP-19 sera. FIG. 14B shows the RSV neutralization titers of anti-VLP-19 sera.

### DESCRIPTION

The present disclosure generally relates to immunogens for eliciting an antibody response against respiratory syncytial virus (RSV). More specifically, the present disclosure relates to virus-like particles (VLPs) including a RSV F protein epitope, as well as methods of use thereof.

### Palivizumab Epitope

An alternative approach to whole virus or RSV subunit vaccines involves the identification of key neutralizing RSV proteins or peptides to which a protective immune response can be generated. In the late 1980s, a mouse monoclonal antibody directed to the fusion protein (F) of RSV was found to have strong RSV neutralizing capability over a broad range of RSV strains (Beeler et al., J Virol, 63:2941-2945, 1989). The highly neutralizing mouse MAb 1129 was subsequently humanized and named palivizumab. Passive transfer of palivizumab (SYNAGIS RSV F protein inhibitor monoclonal antibody manufactured by MedImmune, LLC (Gaithersburg, Md.) has been approved by the U.S. Food and Drug Administration for passive immunization of children for the prevention of serious lower respiratory tract disease caused by RSV. Specifically, safety and efficacy of SYNAGIS was established in children with bronchopulmonary dysplasia, premature infants (birth less than 36 weeks of gestational age), and children with hemodynamically significant congenital heart disease. Palivizumab binds the fusion (F) protein of RSV and neutralizes both genetic subtypes A and B (Blanco et al., Hum Vaccine, 6:482-492, 2012).

The use of palivizumab has not been extended to the general population or adults and is effective prophylactically but not therapeutically. Due to the high cost of antibody prophylaxis, the U.S. is the only country that administers this drug to the majority of high risk infants. Therefore, active vaccination is desirable for controlling RSV.

Because administration of palivizumab is able to reduce the incidence of RSV disease, the epitope targeted by palivizumab is thought to constitute an antigen that could elicit a protective immune response (Impact-RSV Study Group, Pediatrics, 102:531-537, 1998; Meissner et al., Am Acad Ped News, 30:1, 2009; and Wu et al., Curr Top Microbiol Immunol, 317:103-123, 2008). Though the antigen targeted by palivizumab has been studied extensively, there have been difficulties with expressing a sequence in a form that faithfully mimics its presentation in full length RSV F. Some time ago it was determined that the binding site of palivizumab was a contiguous region of F referred to as site A or site II. Attempts were made to generate a peptide vaccine that could elicit a protective immune response. Various 21-, 41- and 61-residue peptides containing the F²⁵⁵⁻²⁷⁵ were tested (Lopez et al., J Gen Virol, 74:2567-2577, 1993). Even though the keyhole limpet haemocyanin-linked peptides could generate antibodies in mice that recognized the peptides, the sera only poorly recognized full length F protein and did not neutralize RSV virus. These results suggest that the peptide acquired a higher order structure in the context of native F but not in the context of the peptide vaccine.

More recently, a peptide library was screened with MAb 19, another RSV neutralizing MAb directed to site A of RSV F (Chargelegue et al., Immunology Letters, 57:15-17, 1997). An 8-mer was detected by MAb 19. When the 8-mer was combined with a Th epitope from measles virus and formulated in a resin, it elicited neutralizing antibodies in mice. The vaccine provided a 77-fold reduction in wild type RSV challenge titer (Chargelegue et al., J Virol, 72:2040-2046, 1998). Interestingly, the mimotope had no sequence homology with native RSV F protein.

There have been reports of limited success with fusing RSV F protein epitopes to various carriers. Specifically, F²⁵⁵⁻²⁷⁸ was fused to cholera toxin, an adjuvant that can elicit a mucosal response with a Th1 bias. Eighty percent of mice immunized intranasally with three doses of the fusion protein in incomplete Freund's adjuvant were protected from challenge with wild type RSV (Singh et al., Viral Immunol, 20:261-275, 2007). Another group expressed the same F protein epitope on capsomeres composed of the L1 capsid protein of human papillomavirus. Though the capsomeres were recognized by antibodies directed to RSV F protein and F protein-specific antibodies were detected in serum from immunized mice, the immune serum was not able to neutralize RSV and no RSV protection data was reported (Murata et al., Virol J, 20:261-275, 2007).

Epitope scaffolds have also been designed to present the motavizumab epitope of the RSV F protein. The peptide scaffolds appeared to have maintained the predicted structure and exposure of key binding sites, and sera from immunized mice had F protein binding activity, but the sera were not able to neutralize RSV (McLellan et al., J Mol Biol, 409:853-866, 2011; and WO 2011/050168 of McLellan et al.).

The innovative approach of the present disclosure expands on the observation that passively administered palivizumab protects infants from severe RSV disease, without causing ERD upon subsequent RSV exposure. This is consistent with reported goals for RSV vaccine development including elicitation of a neutralizing antibody response without inducing Th2 responses associated with ERD (Graham et al., Immunol Rev, 239:149-166, 2011). The B cell site-A epitope on the RSV F glycoprotein recognized by the palivizumab has been well characterized as a 24-residue (F²⁵⁴⁻²⁷⁷) sequential, although conformational, helix-loop-helix (Beeler, J Virol, 63:2941-2945, 1989; Lopez et al., J Gen Virol, 74:2567-2577, 1993; and Arbiza et al., J Gen Virol, 73:2225-2234, 1992). MAbs that bind the palivizumab epitope on the full-length RSV F protein bind to the 24-residue peptide 6000-fold less well (McLellan et al., Nat Struct Biol, 17:248-250, 2009) indicating the conformational nature of the epitope. Furthermore, the epitope is located at a subunit interface in the native trimer, which may explain why such a strong neutralizing epitope is so highly conserved amongst RSV strains, and may constitute a semi-cryptic epitope on the intact virus.

### Woodchuck Hepadnavirus Core Antigen (WHcAg)

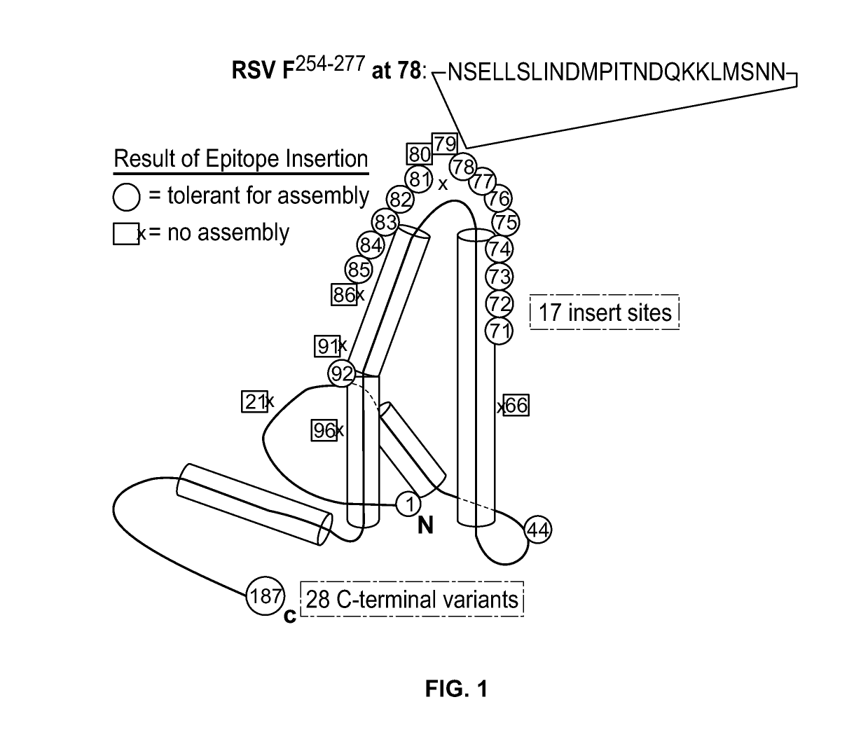

The WHcAg has been chosen as a carrier in part because it is a multimeric, self-assembling, virus-like particle (VLP). The basic subunit of the core particle is a 21 kDa polypeptide monomer that spontaneously assembles into a 240 subunit particulate structure of about 34 nm in diameter. The tertiary and quaternary structures of hepadnavirus core particles have been elucidated (Conway et al., Nature, 386:91-94, 1997) and is shown schematically in FIG. 1. The immunodominant B cell epitope on WHcAg is localized around amino acids 76-82 (Schodel et al., J Exp Med, 180:1037-1046, 1994), which forms a loop connecting adjacent alpha-helices. This observation is consistent with the finding that a heterologous antigen inserted within the 76-82 loop region of HBcAg was significantly more antigenic and immunogenic than the antigen inserted at the N- or C-termini and, importantly, more immunogenic than the antigen in the context of its native protein (Schodel et al., J Virol, 66:106-114, 1992).

The approach of the present disclosure is to genetically insert a polypeptide comprising the palivizumab epitope onto a WHcAg VLP carrier, which will deliver numerous copies of the palivizumab epitope per VLP in a significantly more immunogenic matrix array format than a synthetic peptide. The compositions and methods of the present disclosure involve eliciting palivizumab-like neutralizing antibodies by active immunization, as opposed to the expensive and laborious passive palivizumab immunization. This goal has proven to be practically challenging because the palivizumab epitope is conformational and the inserted epitope must approximate the antigenic structure present on intact RSV. This may explain the failed attempts to present F²⁵⁴⁻²⁷⁷ on other carriers, such as the so-called epitope scaffolds, in a manner suitable for eliciting RSV neutralizing antibodies.

However as discussed herein, the present disclosure has permitted the design and production of a number of hybrid, WHcAg-RSV VLPs that bind palivizumab, elicit high titer neutralizing antibodies and effectively protect mice against a RSV challenge. Without being bound by theory, the success may be partly attributable to the fact that the immunodominant domain of the WHcAg carrier has a helix-loop-helix structure compatible with that of the palivizumab epitope.

### Combinatorial Technology

A problem inherent to the insertion of heterologous epitope sequences into VLP genes is that such manipulation can abolish self-assembly. This assembly problem is so severe that several groups working with the HBcAg or with other VLP technologies (e.g., the L1 protein of the human papillomavirus and Qβ phage) have opted to chemically link the foreign epitopes to the VLPs rather than inserting the epitopes into the particles by recombinant methods. The need to chemically conjugate heterologous antigens has been circumvented by development of a combinatorial technology (Billaud et al., J Virol, 79:13656-13666, 2005). This was achieved by determining 17 different insertion sites and 28 modifications of the WHcAg C-terminus that together favor assembly of chimeric particles, as well as the identification of a number of additional improvements (see, e.g., U.S. Pat. Nos. 7,144,712; 7,320,795; and 7,883,843). ELISA-based screening systems have been developed that measure expression levels, VLP assembly, and insert antigenicity using crude bacterial lysates, avoiding the need to employ labor-intensive purification steps for hybrid VLPs that do not express and/or assemble well.

Several mutations, designated as 42-47 mutations and listed in Table I, were designed to decrease WHcAg-specific antigenicity and/or immunogenicity. The new modified WHcAg carrier platforms provide an advantageous system for presentation of RSV F-protein epitopes.

TABLE I
WHcAg Mutations Affecting Antigenicity and/or Immunogenicity

| Designation | Description |
|---|---|
| Δ1 | WHcAg/insertion of a heterologous antigen |
| | within the immunodominant loop |
| Δ2 | WHcAg/L21A, D26A, L27A, N28A, A29V, V31A |
| | substitutions |
| Δ3 | WHcAg/N136P, A137P substitutions |
| Δ4 | WHcAg/C61S substitution |
| Δ5 | WHcAg/replacement of residues 62-85, 65-88 or |
| | 64-87 with a heterologous antigen |
| Δ6 | WHcAg/R150A, R151A, R152A, R156A, R159A, R162A, |
| | R163A, R164A, R169A, R170A, R171A, R177A, |
| | R178A, R179A, R180A substitutions |
| Δ6.1 | WHcAg/R150A, R151A, R152A, R156A, R159A, R162A, |
| | R163A, R164A, R169A, R170A, R171A substitutions |
| Δ7 | WHcAg/N75A, I76A, T77A, S78A, E79A, Q80A, V81A, |
| | R82A, T83A substitutions |
| Δ7.1 | WHcAg/N75A, I76S, T77S, S78E, E79L, Q80E, V81L, |
| | R82E, T83L substitutions |

### Development of Fusion Proteins and Hybrid Particles

As depicted in FIG. 1, a number of RSV F-protein insertion sites inside the loop region (positions 76-82), as well as outside the loop region were tolerated by WHcAg. Candidate F-protein epitopes were developed based on the epitope profile of the successful palivizumab antibody. The hybrid VLPs of the present disclosure can be grouped into several categories as described in Table II.

TABLE II
Hybrid, WHcAg-hAg VLP Categories

| Category | Description |
|---|---|
| standard | heterologous polypeptide inserted at position |
| | 78 within the immunodominant loop |
| | (e.g., VLP019) |
| epitope- | alterations affecting the heterologous |
| modified | polypeptide (e.g., VLP090) |
| carrier- | alterations affecting the WHcAg carrier |
| modified | (e.g., VLP049) |
| linker- | adding or deleting heterologous polypeptide |
| modified | linkers (e.g., VLP050) |
| varied | insertion of the heterologous polypeptide at |
| position | a position tolerant of assembly other than |
| | position 78 of the WHcAg carrier (e.g., VLP033) |
| replacement | replacement of WHcAg carrier residues with a |
| | heterologous polypeptide (e.g., VLP0123) |

### Antigenic and Immunogenic Characterization of Hybrid, WHcAg-RSV VLPs

### A. Antigenicity

Prior to immunogenicity testing, hybrid WHcAg-RSV VLPs are characterized for expression, particle assembly, and ability to bind a RSV-specific antibody (e.g., palivizumab). The same capture ELISA system used to detect hybrid VLPs in bacterial lysates may be used for purified particles. In brief, expression, particle assembly, and antibody binding are assayed by ELISA. SDS-PAGE and Western blotting may be used to assess the size and antigenicity of each candidate hybrid species.

### B. Immunogenicity

The immune response to hybrid VLPs is assessed. In addition to anti-insert, anti-F-protein and anti-WHcAg antibody endpoint titers, one or more of antibody fine specificity, isotype distribution, antibody persistence and antibody avidity are monitored. Examples of these assays are described below. Immune responses are tested in vivo in various mammalian species (e.g., rodents such as mice and cotton rats, nonhuman primates, humans, etc.).

### Compositions

The compositions of the present disclosure comprise a hybrid woodchuck hepadnavirus core antigen or a polynucleotide encoding the hybrid core antigen, wherein the hybrid core antigen is a fusion protein comprising a respiratory syncytial virus (RSV) F polypeptide and a woodchuck hepadnavirus core antigen, and wherein the fusion protein is capable of assembling as a hybrid virus-like particle (VLP). In some embodiments, the RSV F polypeptide comprises a palivizumab epitope (e.g., capable of being bound by palivizumab). In preferred embodiments, the composition is an antigenic composition. In some embodiments, the composition further comprises a pharmaceutically acceptable carrier. The term “carrier” refers to a vehicle within which the hybrid core antigen or polynucleotide encoding the antigen is administered to a mammalian subject. The term carrier encompasses diluents, excipients, adjuvants and combinations thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers. Exemplary “excipients” are inert substances include but are not limited to polymers (e.g., polyethylene glycol), carbohydrates (e.g., starch, glucose, lactose, sucrose, cellulose, etc.), and alcohols (e.g., glycerol, sorbitol, xylitol, etc.).

Adjuvants are broadly separated into two classes based upon their primary mechanism of action: vaccine delivery systems (e.g., emulsions, microparticles, iscoms, liposomes, etc.) that target associated antigens to antigen presenting cells (APC); and immunostimulatory adjuvants (e.g., LPS, MLP, CpG, etc.) that directly activate innate immune responses. The WHcAg platform provides a delivery system that targets antigen specific B cells and other primary APC, as well as efficient T cell help for antigen-specific B cells. Briefly, the WHcAg platform functions as an immunostimulatory adjuvant by directly activating antigen-specific B cells by virtue of cross-linking membrane immunoglobulin (mIg) receptors for induction of B7.1 and B7.2 costimulatory molecule expression on naive resting B cells (Milich et al., Proc Natl Acad Sci USA, 94:14648-14653, 1997). Additionally, hepadnaviral core particles contain a protamine-like sequence that binds ssRNA, which acts as a TLR7 ligand (Lee et al., J Immunol, 182:6670-6681, 2009)

### A. Traditional and Molecular Adjuvants

Although adjuvants are not required when using the WHcAg delivery system, some embodiments of the present disclosure employ traditional and/or molecular adjuvants. Specifically, immunization in saline effectively elicits anti-insert antibody production. However, formulation in non-inflammatory agents such as mineral oil, squalene, and aluminum salts (e.g., aluminum hydroxide, aluminum phosphate, etc.), enhance immunogenicity. Importantly, administration of WHcAg results in the production of all four IgG isotypes, regardless of which if any adjuvant is employed. Inclusion of a CpG motif also enhances the primary response. Moreover, use of an inflammatory adjuvant such as the Ribi formulation is not more beneficial than is the use of non-inflammatory adjuvants, indicating that the benefits of the adjuvants result from a depot effect rather than from non-specific inflammation. Thus, the core platform is used with no adjuvant or with non-inflammatory adjuvants depending upon the application and the quantity of antibody desired. In some embodiments of the present disclosure, IFA is used in murine studies, whereas alum or squalene is used in human studies. In instances where it is desirable to deliver hybrid WHcAg particles in a single dose in saline, a molecular adjuvant is employed. A number of molecular adjuvants are employed to bridge the gap between innate and adaptive immunity by providing a co-stimulus to target B cells or other APCs.

### B. Other Molecular Adjuvants

Genes encoding the murine CD40L (both 655 and 470 nucleic acid versions) have been used successfully to express these ligands at the C-terminus of WHcAg (See, WO 2005/011571). Moreover, immunization of mice with hybrid WHcAg-CD40L particles results in the production of higher anti-core antibody titers than does the immunization of mice with WHcAg particles. However, lower than desirable yields of purified particles have been obtained. Therefore, mosaic particles containing less than 100% CD40L-fused polypeptides are produced to overcome this problem. The other molecular adjuvants inserted within the WHcAg, including the C3d fragment, BAFF and LAG-3, have a tendency to become internalized when inserted at the C-terminus. Therefore tandem repeats of molecular adjuvants are used to resist internalization. Alternatively, various mutations within the so-called hinge region of WHcAg, between the assembly domain and the DNA/RNA-binding region of the core particle are made to prevent internalization of C-terminal sequences. However, internalization represents a problem for those molecular adjuvants such as CD40L, C3d, BAFF and LAG-3, which function at the APC/B cell membrane. In contrast, internalization of molecular adjuvants such as CpG DN is not an issue as these types of adjuvants function at the level of cytosolic receptors.

Another type of molecular adjuvant or immune enhancer is the inclusion within hybrid core particles of a CD4+ T cell epitope, preferably a “universal” CD4+ T cell epitope that is recognized by a large proportion of CD4+ T cells (such as by more than 50%, preferably more than 60%, more preferably more than 70%, most preferably greater than 80%), of CD4+ T cells. In one embodiment, universal CD4+ T cell epitopes bind to a variety of human MHC class II molecules and are able to stimulate T helper cells. In another embodiment, universal CD4+ T cell epitopes are preferably derived from antigens to which the human population is frequently exposed either by natural infection or vaccination (Falugi et al., Eur J Immunol, 31:3816-3824, 2001). A number of such universal CD4+ T cell epitopes have been described including, but not limited to: Tetanus Toxin (TT) residues 632-651; TT residues 950-969; TT residues 947-967, TT residues 830-843, TT residues 1084-1099, TT residues 1174-1189 (Demotz et al., Eur J Immunol, 23:425-432, 1993); Diphtheria Toxin (DT) residues 271-290; DT residues 321-340; DT residues 331-350; DT residues 411-430; DT residues 351-370; DT residues 431-450 (Diethelm-Okita et al., J Infect Dis, 1818:1001-1009, 2000); Plasmodium falciparum circumsporozoite (CSP) residues 321-345 and CSP residues 378-395 (Hammer et al., Cell, 74:197-203, 1993); Hepatitis B antigen (HBsAg) residues 19-33 (Greenstein et al., J Immunol, 148:3970-3977, 1992); Influenza hemagglutinin residues 307-319; Influenza matrix residues 17-31 (Alexander et al., J Immunol, 164:1625-1633, 2000); and measles virus fusion protein (MVF) residues 288-302 (Dakappagari et al., J Immunol, 170:4242-4253, 2003).

### Methods of Inducing an Immune Response

The present disclosure provides methods for eliciting an immune response in an animal in need thereof, comprising administering to the animal an effective amount of an antigenic composition comprising a hybrid woodchuck hepadnavirus core antigen, wherein the hybrid core antigen is a fusion protein comprising a respiratory syncytial virus (RSV) F polypeptide (e.g., palivizumab epitope) and a woodchuck hepadnavirus core antigen, and wherein said fusion protein assembles as a hybrid virus-like particle (VLP). Also provided by the present disclosure are methods for eliciting an immune response in an animal in need thereof, comprising administering to the animal an effective amount of an antigenic composition comprising a polynucleotide encoding a hybrid woodchuck hepadnavirus core antigen, wherein the hybrid core antigen is a fusion protein comprising a RSV F polypeptide and a woodchuck hepadnavirus core antigen, and wherein said fusion protein assembles as a hybrid virus-like particle (VLP). Unless otherwise indicated, the antigenic composition is an immunogenic composition.

The immune response raised by the methods of the present disclosure generally includes an antibody response, preferably a neutralizing antibody response, preferably a protective antibody response. Methods for assessing antibody responses after administration of an antigenic composition (immunization or vaccination) are well known in the art. In some embodiments, the immune response comprises a T cell-mediated response (e.g., RSV F-specific response such as a proliferative response, a cytokine response, etc.). In preferred embodiments, the immune response comprises both a B cell and a T cell response. Antigenic compositions can be administered in a number of suitable ways, such as intramuscular injection, subcutaneous injection, and intradermal administration. Additional modes of administration include but are not limited to intranasal administration, and oral administration.

Antigenic compositions may be used to treat both children and adults, including pregnant women. Thus a subject may be less than 1 year old, 1-5 years old, 5-15 years old, 15-55 years old, or at least 55 years old. Preferred subjects for receiving the vaccines are the elderly (e.g., >55 years old, >60 years old, preferably >65 years old), and the young (e.g., <6 years old, 1-5 years old, preferably less than 1 year old).

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Administration of more than one dose (typically two doses) is particularly useful in immunologically naive subjects or subjects of a hyporesponsive population (e.g., diabetics, subjects with chronic kidney disease, etc.). Multiple doses will typically be administered at least 1 week apart (e.g., about 2 weeks, about 3 weeks, about 4 weeks, about 6 weeks, about 8 weeks, about 10 weeks, about 12 weeks, about 16 weeks, and the like.). Preferably multiple doses are administered from one, two, three, four or five months apart. Antigenic compositions of the present disclosure may be administered to patients at substantially the same time as (e.g., during the same medical consultation or visit to a healthcare professional) other vaccines.

In general, the amount of protein in each dose of the antigenic composition is selected as an amount effective to induce an immune response in the subject, without causing significant, adverse side effects in the subject. Preferably the immune response elicited is a neutralizing antibody, preferably a protective antibody response. Protective in this context does not necessarily mean the subject is completely protected against infection, rather it means that the subject is protected from developing symptoms of disease, especially severe disease associated with the pathogen corresponding to the heterologous antigen.

The amount of hybrid core antigen (e.g., VLP) can vary depending upon which antigenic composition is employed. Generally, it is expected that each human dose will comprise 1-1500 μg of protein (e.g., hybrid core antigen), such as from about 1 μg to about 1000 μg, for example, from about 1 μg to about 500 μg, or from about 1 μg to about 100 μg. In some embodiments, the amount of the protein is within any range having a lower limit of 10, 20, 30, 40, 50, 60, 70, 80, 90, 100, 110, 120, 130, 140, 150, 160, 170, 180, 190, 200, 210, 220, 230, 240 or 250 μg, and an independently selected upper limit of 1000, 950, 900, 850, 800, 750, 700, 650, 600, 550, 500, 450, 400, 350, 300 or 250 μg, provided that the lower limit is less than the upper limit. Generally a human dose will be in a volume of from 0.1 ml to 1 ml, preferably from 0.25 ml to 0.5 ml. The amount utilized in an immunogenic composition is selected based on the subject population. An optimal amount for a particular composition can be ascertained by standard studies involving observation of antibody titers and other responses (e.g., antigen-induced cytokine secretion) in subjects. Following an initial vaccination, subjects can receive a boost in about 4-12 weeks.

### Kits

Also provided by the present disclosure are kits comprising a hybrid woodchuck hepadnavirus core antigen and a woodchuck hepadnavirus core antigen, wherein the hybrid core antigen is a fusion protein comprising a respiratory syncytial virus (RSV) F polypeptide (e.g., palivizumab epitope) and a woodchuck hepadnavirus core antigen, and wherein said fusion protein assembles as a hybrid virus-like particle (VLP), and wherein the core antigen lacks the RSV F polypeptide. In some embodiments, the kits further comprise instructions for measuring RSV F polypeptide-specific antibodies. In some embodiments, the antibodies are present in serum from a blood sample of a subject immunized with an antigenic composition comprising the hybrid woodchuck hepadnavirus core antigen.

As used herein, the term “instructions” refers to directions for using reagents (e.g., hybrid core antigen and core antigen) contained in the kit for measuring antibody titer. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination.

### DEFINITIONS

As used herein, the singular forms “a”, “an”, and “the” include plural references unless indicated otherwise. For example, “an” excipient includes one or more excipients. The term “plurality” refers to two or more.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Current Protocols in Molecular Biology (Ausubel et al., eds., 1987); PCR: The Polymerase Chain Reaction, (Mullis et al., eds., 1994); Culture of Animal Cells: A Manual of Basic Technique (Freshney, 1987); Harlow et al., Antibodies: A Laboratory Manual (Harlow et al., 1988); and Current Protocols in Immunology (Coligan et al., eds., 1991).

The terms “F protein,” “Fusion protein” and “F polypeptide” refer to a respiratory syncytial virus (RSV) RSV fusion glycoprotein. Numerous RSV F proteins have been described and are known to those of skill in the art. An exemplary F protein is set forth in GENBANK Accession No. AAB59858.1.

As used herein, the terms “virus-like particle” and “VLP” refer to a structure that resembles a virus. VLPs of the present disclosure lack a viral genome and are therefore noninfectious. Preferred VLPs of the present disclosure are woodchuck hepadnavirus core antigen (WHcAg) VLPs.

The terms “hybrid” and “chimeric” as used in reference to a hepadnavirus core antigen, refer to a fusion protein of the hepadnavirus core antigen and an unrelated antigen (e.g., RSV F polypeptide such as one or more of SEQ ID NO:3, 86-114, and variants thereof). For instance, in some embodiments, the term “hybrid WHcAg” refers to a fusion protein comprising both a WHcAg component (full length, or partial) and a heterologous antigen or fragment thereof.

The term “heterologous” with respect to a nucleic acid, or a polypeptide, indicates that the component occurs where it is not normally found in nature and/or that it originates from a different source or species.

An “effective amount” or a “sufficient amount” of a substance is that amount necessary to effect beneficial or desired results, including clinical results, and, as such, an “effective amount” depends upon the context in which it is being applied. In the context of administering an immunogenic composition, an effective amount contains sufficient antigen (e.g., hybrid, WHcAg-RSV F VLP) to elicit an immune response (preferably a measurable level of RSV-neutralizing antibodies). An effective amount can be administered in one or more doses.

The term “dose” as used herein in reference to an immunogenic composition refers to a measured portion of the immunogenic composition taken by (administered to or received by) a subject at any one time.

The term “about” as used herein in reference to a value, encompasses from 90% to 110% of that value (e.g., about 20 μg VLP refers to 1.8 μg to 22 μg VLP).

As used herein the term “immunization” refers to a process that increases an organisms' reaction to antigen and therefore improves its ability to resist or overcome infection.

The term “vaccination” as used herein refers to the introduction of vaccine into a body of an organism.

A “variant” when referring to a polynucleotide or a polypeptide (e.g., an RSV F polynucleotide or polypeptide) is a polynucleotide or a polypeptide that differs from a reference polynucleotide or polypeptide. Usually, the difference(s) between the variant and the reference constitute a proportionally small number of differences as compared to the reference (e.g., at least 90%, 91%, 92%, 93%, 94%, 95%, 96%, 97%, 98% or 99% identical). In some embodiments, the present disclosure provides hybrid WHcAg-RSV F VLPs having at least one addition, insertion or substitution in one or both of the WHcAg or RSV F portion of the VLP.

The term “wild type” when used in reference to a polynucleotide or a polypeptide refers to a polynucleotide or a polypeptide that has the characteristics of that polynucleotide or a polypeptide when isolated from a naturally-occurring source. A wild type polynucleotide or a polypeptide is that which is most frequently observed in a population and is thus arbitrarily designated as the “normal” form of the polynucleotide or a polypeptide.

Amino acids may be grouped according to common side-chain properties: hydrophobic (Met, Ala, Val, Leu, Ile); neutral hydrophilic (Cys, Ser, Thr, Asn, Gln); acidic (Asp, Glu); basic (His, Lys, Arg); aromatic (Trp, Tyr, Phe); and orientative (Gly, Pro). Another grouping of amino acids according to side-chain properties is as follows: aliphatic (glycine, alanine, valine, leucine, and isoleucine); aliphatic-hydroxyl (serine and threonine); amide (asparagine and glutamine); aromatic (phenylalanine, tyrosine, and tryptophan); acidic (glutamic acid and aspartic acid); basic (lysine, arginine, and histidine); sulfur (cysteine and methionine); and cyclic (proline). In some embodiments, the amino acid substitution is a conservative substitution involving an exchange of a member of one class for another member of the same class. In other embodiments, the amino acid substitution is a non-conservative substitution involving an exchange of a member of one class for a member of a different class.

The percent identity between the two sequences is a function of the number of identical positions shared by the sequences, taking into account the number of gaps, and the length of each gap, which need to be introduced for optimal alignment of the two sequences. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1.

A “recombinant” nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques. A “recombinant” protein is one that is encoded by a heterologous (e.g., recombinant) nucleic acid, which has been introduced into a host cell, such as a bacterial or eukaryotic cell. The nucleic acid can be introduced, on an expression vector having signals capable of expressing the protein encoded by the introduced nucleic acid or the nucleic acid can be integrated into the host cell chromosome.

An “antigen” is a compound, composition, or substance that can stimulate the production of antibodies and/or a T cell response in a subject, including compositions that are injected, absorbed or otherwise introduced into a subject. The term “antigen” includes all related antigenic epitopes. The term “epitope” or “antigenic determinant” refers to a site on an antigen to which B and/or T cells respond. The “dominant antigenic epitopes” or “dominant epitope” are those epitopes to which a functionally significant host immune response, e.g., an antibody response or a T-cell response, is made. Thus, with respect to a protective immune response against a pathogen, the dominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

“Adjuvant” refers to a substance which, when added to a composition comprising an antigen, nonspecifically enhances or potentiates an immune response to the antigen in the recipient upon exposure. Common adjuvants include suspensions of minerals (alum, aluminum hydroxide, aluminum phosphate) onto which an antigen is adsorbed; emulsions, including water-in-oil, and oil-in-water (and variants thereof, including double emulsions and reversible emulsions), liposaccharides, lipopolysaccharides, immunostimulatory nucleic acids (such as CpG oligonucleotides), liposomes, Toll-like Receptor agonists (particularly, TLR2, TLR4, TLR7/8 and TLR9 agonists), and various combinations of such components.

An “antibody” or “immunoglobulin” is a plasma protein, made up of four polypeptides that binds specifically to an antigen. An antibody molecule is made up of two heavy chain polypeptides and two light chain polypeptides (or multiples thereof) held together by disulfide bonds. In humans, antibodies are defined into five isotypes or classes: IgG, IgM, IgA, IgD, and IgE. IgG antibodies can be further divided into four sublclasses (IgG1, IgG2, IgG3 and IgG4). A “neutralizing” antibody is an antibody that is capable of inhibiting the infectivity of a virus. Accordingly, a neutralizing antibodies specific for RSV are capable of inhibiting or reducing the infectivity of RSV.

An “immunogenic composition” is a composition of matter suitable for administration to a human or animal subject (e.g., in an experimental or clinical setting) that is capable of eliciting a specific immune response, e.g., against a pathogen, such as RSV. As such, an immunogenic composition includes one or more antigens (for example, polypeptide antigens) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or reduced or ameliorated) by inhibiting replication of the pathogen (e.g., RSV) following exposure of the subject to the pathogen. In the context of this disclosure, the term immunogenic composition will be understood to encompass compositions that are intended for administration to a subject or population of subjects for the purpose of eliciting a protective or palliative immune response against RSV (that is, vaccine compositions or vaccines).

An “immune response” is a response of a cell of the immune system, such as a B cell, T cell, or monocyte, to a stimulus, such as a pathogen or antigen (e.g., formulated as an immunogenic composition or vaccine). An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo. Exposure of a subject to an immunogenic stimulus, such as a pathogen or antigen (e.g., formulated as an immunogenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an immunogenic composition increases the magnitude of an antigen (or pathogen) specific response, (e.g., by increasing antibody titer and/or affinity, by increasing the frequency of antigen specific B or T cells, by inducing maturation effector function, or any combination thereof).

The term “reduces” is a relative term, such that an agent reduces a response or condition if the response or condition is quantitatively diminished following administration of the agent, or if it is diminished following administration of the agent, as compared to a reference agent. Similarly, the term “protects” does not necessarily mean that an agent completely eliminates the risk of an infection or disease caused by infection, so long as at least one characteristic of the response or condition is substantially or significantly reduced or eliminated. Thus, an immunogenic composition that protects against or reduces an infection or a disease, or symptom thereof, can, but does not necessarily prevent or eliminate infection or disease in all subjects, so long as the incidence or severity of infection or incidence or severity of disease is measurably reduced, for example, by at least about 50%, or by at least about 60%, or by at least about 70%, or by at least about 80%, or by at least about 90% of the infection or response in the absence of the agent, or in comparison to a reference agent. In certain instances, the reduction is in the incidence of lower respiratory tract infections (LRTI), or the incidence of severe LRTI, or hospitalizations due to RSV disease, or in the severity of disease caused by RSV.

A “subject” is a living multi-cellular vertebrate organism. In the context of this disclosure, the subject can be an experimental subject, such as a non-human animal (e.g., a mouse, a rat, or a non-human primate). Alternatively, the subject can be a human subject.

The terms “derived from” or “of” when used in reference to a nucleic acid or protein indicates that its sequence is identical or substantially identical to that of an organism of interest.

The terms “decrease,” “reduce” and “reduction” as used in reference to biological function (e.g., enzymatic activity, production of compound, expression of a protein, etc.) refer to a measurable lessening in the function by preferably at least 10%, more preferably at least 50%, still more preferably at least 75%, and most preferably at least 90%. Depending upon the function, the reduction may be from 10% to 100%. The term “substantial reduction” and the like refers to a reduction of at least 50%, 75%, 90%, 95% or 100%.

The terms “increase,” “elevate” and “elevation” as used in reference to biological function (e.g., enzymatic activity, production of compound, expression of a protein, etc.) refer to a measurable augmentation in the function by preferably at least 10%, more preferably at least 50%, still more preferably at least 75%, and most preferably at least 90%. Depending upon the function, the elevation may be from 10% to 100%; or at least 10-fold, 100-fold, or 1000-fold up to 100-fold, 1000-fold or 10,000-fold or more. The term “substantial elevation” and the like refers to an elevation of at least 50%, 75%, 90%, 95% or 100%.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the bacteria that produced the protein. As such an isolated protein is free of extraneous compounds (e.g., culture medium, bacterial components, etc.).

### EXAMPLES

Abbreviations: BSA (bovine serum albumin); ELISA (enzyme-linked immunosorbent assay); ERD (enhanced respiratory disease); FI (formalin-inactivated); IFA (incomplete Freund's adjuvant); MAb, or mAb (monoclonal antibody); OD (optical density); PBS (phosphate buffered saline); pfu or PFU (plaque forming units); PRNT (plaque reduction neutralization titer); RSV (respiratory syncytial virus); sF (soluble RSV F protein); VLP (virus-like particles); and WHcAg (woodchuck hepadnavirus core antigen).

### Example 1

### Hybrid Woodchuck Hepadnavirus Core Antigen-Respiratory Syncytial Virus F Polypeptide Virus-Like Particles

This example provides exemplary methods for producing and characterizing hybrid, WHcAg-RSV VLPs. Briefly, WHcAg-RSV VLPs were constructed from known and putative epitopes of therapeutic RSV monoclonal antibodies. The hybrid VLPs were tested for antigenicity, and immunogenicity. Hybrid VLPs were also tested for the ability to elicit RSV-neutralizing antibodies.

### Materials and Methods

Construction and Expression of Recombinant Hybrid WHcAg Particles.

The woodchuck hepatitis virus genome has previously been described (Cohen et al., Virology, 162:12-20, 1998), GENBANK Accession No. NC_{—}004107 (SEQ ID NO:4). Full length WHcAg (188 amino acids) was expressed from the pUC-WHcAg vector under the control of the Lac operon promoter. RSV F sequences were either designed to contain unique enzyme restriction sites or overlapping oligonucleotides were designed to insert the RSV sequences into the pUC-WHcAg vector (Billaud et al., J Virol, 79:13656-13666, 2005; and Billaud et al., Vaccine 25:1593-1606, 2007). For the RSV-fused and the RSV-replacement sequences, insertion was achieved by PCR using overlapping oligonucleotides. For VLPs inserted at position 76, 78, 81 and 82, the restriction sites EcoRI and XhoI were used, which resulted in the inclusion of N-terminal and C-terminal linkers flanking the heterologous polypeptide insert. Thus, the standard linker combination of the VLPs of the present disclosure is GILE-Xn-L, where X is any amino acid, and n is 60 or less (SEQ ID NO:5). For VLPs inserted at position 74, an existing SacI restriction site was used. C-terminal fusion was achieved by adding the EcoRV restriction site, which adds aspartic acid and isoleucine at the junction. N-terminal fusion was achieved by adding an NcoI restriction site upstream of the WLWG linker (SEQ ID NO:6).

Some of the hybrid WHcAg-RSV VLPs were constructed on full length (SEQ ID NO:1) or truncated WHcAg cores (SEQ ID NO:2), while others were constructed on full length or truncated WHcAg cores comprising modifications. Some WHcAg modifications were previously described in U.S. Pat. No. 7,320,795. Other WHcAg modifications were made so as to reduce carrier-specific antigenicity, and include:

Δ2-WHcAg, Δ3-WHcAg, Δ4-WHcAg, Δ5-WHcAg, Δ6-WHcAg, Δ6.1-WHcAg, Δ7-WHcAg, and Δ7.1-WHcAg (described above in Table I).

Plasmids were transformed into chemically competent TOP10 or DH5alpha E. coli host cells according to the manufacturer's protocol. The bacteria were grown overnight then lysed in a lysozyme-salt solution and clarified by centrifugation at 20,000×G for 30 min. The resulting supernatant was precipitated overnight in the cold with 25% ammonium sulfate. Lysates were screened in capture enzyme-linked immunosorbent assays (ELISAs) designed to assess three properties of each VLP: 1) protein expression of the WHcAg polypeptide by use of the 2221 MAb (Institute for Immunology, Tokyo University, Japan) specific for an epitope within residues 129 to 140 of WHcAg; 2) particle assembly using an antibody specific for a conformational epitope on WHcAg; and 3) display and correct conformation of the RSV site A epitope by use of palivizumab. The capture antibody was peptide-specific and noncompetitive with the detecting antibodies. The constructs that were positive for all three properties were selected for further purification on hydroxyapatite followed by gel filtration chromatography on SEPHAROSE 4B columns. The size and antigenicity of each hybrid, WHcAg-RSV F protein was confirmed by SDS-PAGE and Western blotting. Yields generally exceeded 75 mg/L. The hybrid WHcAg-RSV VLP, VLP-19, was also assessed by cryoelectron microscopy.

ELISA Assay.

High binding ELISA plates (Costar) were coated overnight with 10 μg/ml peptide, 1 μg/ml of VLP or 0.2 μg/ml soluble RSV F (sF). In a further study, ELISA plates were coated overnight with 0.2 μg/ml of test VLP, VLP-19, soluble RSV F (sF) as a positive control, or WHcAg as a negative control. Plates were blocked with SuperBlock (Thermoscientific) or 3% BSA in PBS. Five-fold dilutions of mouse antisera or palivizumab (starting at 1 mg/ml), or two-fold dilutions of human plasma samples were made, and 50 μl per well was applied to the plates for 1 hr. In a further study, palivizumab or human plasma samples were diluted two-fold in SuperBlock, and 50 μl per well was applied to the plates for 1 hr. After four washes in 0.5% Tween 20 in PBS buffer, HRP-conjugated secondary anti-human IgG Ab or anti-mouse IgG Ab diluted 1:5000 was applied for 1 hr. After washing, color was developed with 100 μl per well tetramethylbenzidine (Sigma). The reaction was stopped by addition of 100 per well 0.1 N HCl and optical density (OD) at 450 nm was read on an ELISA plate reader. The OD for sF-coated wells was between 1.5 and 2.5. For IgG isotype analysis, secondary antibodies specific for the various IgG isotypes were used.

For competition ELISA assay with VLPs, a constant concentration of 100 ng/ml palivizumab was mixed with five-fold or two-fold dilutions of VLP (e.g., VLP19, WHcAg carrier or sF), and the mixture was applied to the ELISA wells. Detection was performed with HRP-conjugated anti-human antibody to detect bound palivizumab. Data were plotted as % inhibition. For mouse serum ELISA assays, two-fold dilutions of mouse serum were prepared in 3% BSA in PBS. The endpoint titer was calculated as the highest dilution with an OD two-fold greater than the blank. For competition ELISA with anti-VLP serum, five-fold or two-fold dilutions of anti-VLP-19 or control serum were mixed with 10 ng/ml palivizumab and applied to sF-coated ELISA plates. Detection was performed with HRP-conjugated anti-human antibody. For calculating percent binding, VLP-coated and sF-coated wells to which 0.5 μg/ml palivizumab had been applied were compared. The OD for sF-coated wells was set at 100%, and the ODs for the VLP-coated wells were calculated relative to the 100% mark.

SDS PAGE and Western Blotting.

1 μg of material was separated in a 12% SDS PAGE Tris-glycine gel that was stained with Sypro Ruby (Invitrogen). Duplicate gels were transferred to PVDF membranes (Invitrogen) and probed with either palivizumab (MedImmune) followed by HRP-conjugated anti-human Ab (Dako) or with anti-WHc rabbit Ab (VLP Biotech) followed by HRP-conjugated anti-rabbit HRP (Dako). Signal was developed with an electrochemiluminescence solution (Thermoscientific). Bands were visualized on a GE ImageQuant LAS 4000 analyzer.

Electron Microscopy (EM).

At NanoImaging Services, Inc., cryoelectron microscopy analysis was performed on WHcAg, VLP-19 and VLP-19 with palivizumab Fabs in PBS buffer. Palivizumab Fabs were generated with an Immunopure Fab kit (Thermoscientific). 20 μL of VLP-19 at 1.0 mg/ml were mixed with 20 μL of Fabs at 1.0 mg/ml in PBS buffer and incubated 4 hr at RT prior to EM analysis. Briefly, a 3 μL drop of sample buffer was applied to a holey carbon film on a 400-mesh copper grid and vitrified in liquid ethane. The grids were stored under liquid nitrogen prior to imaging with an FEI Tecnai T12 electron microscope, operating at 120 keV equipped with an FEI Eagle 4k×4k CCD camera at <170 C.

Mouse Immunization and Challenge.

For immunogenicity testing, B10xB10.S F1 mice were immunized intraperitoneally with 20 μg of VLP emulsified in IFA and boosted at week 8 with 10 μg in IFA. For RSV challenge experiments Balb/c mice were immunized intramuscularly on days 0 and 14 with 100 μg VLP with 250 μg alum, 40 μg with a 0.5% oil-in-water emulsion or 20 μg emulsified in IFA. On day 28, serum was collected and mice were challenged with 10⁶ PFU of wild type RSV (wtRSV) in 10 μl delivered intranasally. Four days post challenge, lung tissue was harvested, weighed, and titered by plaque assay.

In a further study, female Balb/c mice, 6-8 weeks of age were randomly divided into cohorts of five and consecutively numbered in the animal care facility at MedImmune according to IACUC procedures. On days 0 and 14, mice were immunized intramuscularly with 50 μl of 40 μg of the VLP to be tested or PBS in 50 μl Imject IFA (Pierce). A final cohort of mice received one dose of 10⁶ PFU wtRSV-A2 delivered intranasally in 100 μl on day 0. On day 28, sera were collected and mice were challenged with 10⁶ PFU of wtRSV-A2 delivered intranasally in 100 μl. Four days post challenge, lung tissue was harvested, kept on ice, weighed, and homogenized in 2 ml optiMEM within three hours of harvest. Following a low speed spin at 1500 rpm for five minutes, the lung supernatants were titered by plaque assay.

Five mice were included in each cohort because with a normal distribution and expected standard deviation of ≦0.5, five data points are expected to be sufficient to discern whether VLP-immunization provided protection by reducing RSV lung titers by two or more log 10 compared to a placebo titer of approximately four log 10 PFU/g.

In a separate study, 35 mice were immunized with four doses at two week intervals with 20 μg/dose VLP-19 formulated in a proprietary adjuvant. Two weeks following the final dose, sera were collected from all mice and combined.

Cotton Rat Immunization and Challenge.

For cotton rat RSV challenge experiments, animals were immunized intramuscularly on weeks 0, 4 and 7 with 0.5 μg sF protein with 250 μg alum, 100 μg VLP-19 with 250 μg alum or PBS. At week 10, serum was collected and cotton rats were challenged with 10⁶ PFU of wild type RSV in 10 μl delivered intranasally. Four days post challenge, lung tissue was harvested, weighed, and titered by plaque assay.

RSV Plaque Assay.

Dilutions of virus in lung samples were made in optiMEM. Ten-fold, hundred-fold and thousand-fold dilutions of virus were applied to monolayers of Vero cells TC6-well plates. Vero cells were purchased from ATCC and tested for mycoplasma in a MedImmune cell culture facility. After 1 hr, the inoculum was replaced with methylcellulose-supplemented-medium (2% methylcellulose mixed 1:1 with 2×L-15/EMEM [SAFC] supplemented with 2% fetal bovine serum, 4 mM L-glutamine and 200 U penicillin with 200 μg/ml streptomycin [Gibco]) and incubated at 35° C. for 4-5 days. Overlay was aspirated and cells were immunostained with goat anti-RSV antibody (Chemicon 1128) followed by HRP-conjugated anti-goat antibody (Dako). Red colored plaques were developed with 3-amino-9-ethylcarbazole (Dako). Titer was recorded as plaque forming units (PFU)/gram lung tissue.

RSV PRNT Neutralization Assay.

Serum was heat-inactivated at 56° C. for 50 minutes. Dilutions of serum were combined with 150 PFU (100-200 PFU) of RSV in optiMEM and incubated at 35° C. for 1 hr before applying to 80% confluent monolayers of Vero cells in TC6-well plates. Cells were incubated with the serum-virus mixture for 1 hr. The inoculum was aspirated and cells were overlaid with methylcellulose-supplemented-medium, incubated for 5 days and immunostained with goat anti-RSV antibody (Chemicon 1128) followed by HRP-conjugated anti-goat antibody (Dako). Red colored plaques were developed with 3-amino-9-ethylcarbazole (Dako). The plaque reduction neutralization titer (PRNT) was calculated as the dilution at which 50% of RSV was neutralized compared to controls incubated in the absence of serum.

RSV Microneutralization Assay.

Serum was heat-inactivated at 56° C. for 50 minutes. Dilutions of serum were combined with 500 PFU of GFP-expressing RSV (RSV/GFP) and incubated at 33° C. for 1 hr before applying to monolayers of Vero cells in 96-well plates in triplicate. After incubation for 22 hr, fluorescent foci units (FFU) were enumerated by an Isocyte imager. The reported neutralization titer is the interpolated dilution at which 50% of the input RSV/GFP virus was neutralized.

### Results

Numerous Hybrid, WHcAg-RSV VLPs were Designed and Tested.

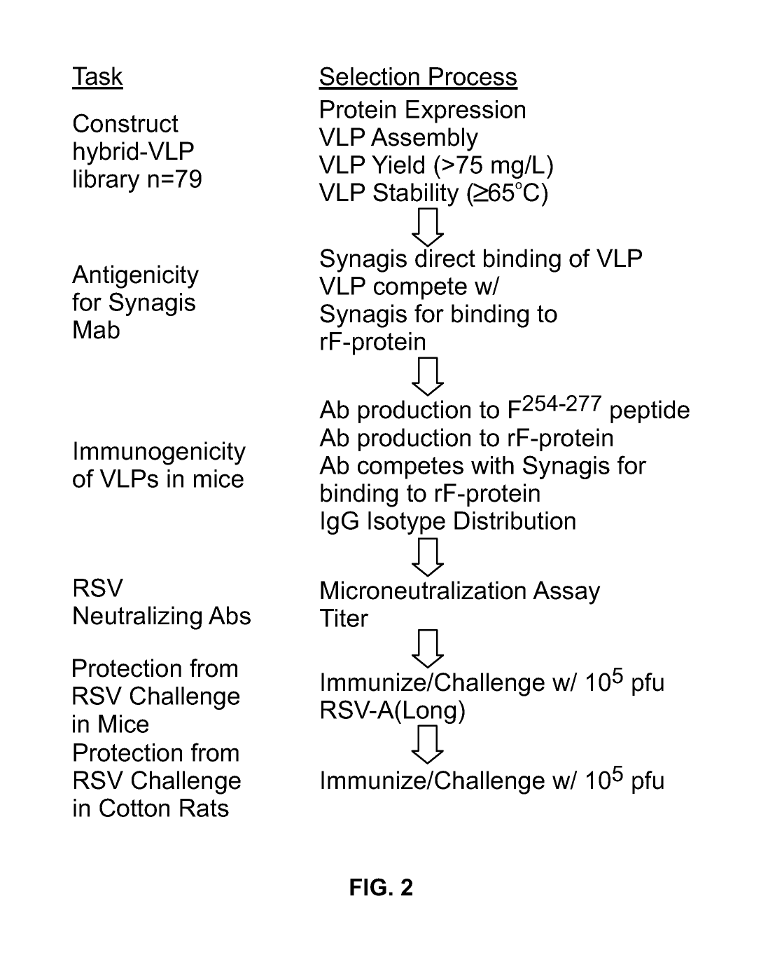

A schematic of the WHcAg structure as a carrier for heterologous polypeptides, such as RSV F fragments comprising a B cell epitope is provided as FIG. 1. A flow chart of exemplary methods for selecting immunogenic, hybrid, WHcAg-RSV VLPs is provided as FIG. 2, and results are enumerated in Table 1-0. A summary of hybrid, WHcAg-RSV VLPs that designed and tested during development of the present disclosure is provided as Table 1-1A. The hybrid VLPs that do not bind and encapsidate ssRNA (e.g., TLR7 adjuvant) include: VLP018, VLP021.1, VLP029, VLP031, VLP041, VLP046, VLP051, VLP078, VLP081, and VLP087. A summary of the RSV F polypeptide inserts of the hybrid VLPs is provided as Table 1-B.

TABLE 1-0
Hybrid, WHcAg-RSV VLP Summary

| Task | Selection Criteria | Outcome |
|---|---|---|
| Design | WHcAg plus RSV F epitope | 79 designed |
| Construction | Assembly, Yield and Stability | 55 assembled |
| Antigenicity | Palivizumab Binds VLP | 54 bound palivizumab |
| Immunogen- | Serum Binds rF protein | 52 elicited high titer |
| icity | | Abs |
| RSV | Microneutralization Assay | 20 elicited intermediate |
| Neutralizing | | to high titer neutralizing |
| Abs* | | Abs |
| Protection | Challenge Immunized Mice | 18 provided intermediate |
| from RSV** | with 10⁶ PFU RSV A2 Strain | to high levels of |
| | | protection |

*RSV neutralizing titers were categorized based on average Log2 values: high ≧7; and intermediate <7 and ≦5.
**Protection is defined by log10 reductions in RSV lung titer: high ≧2; and intermediate <2 and ≧1.

TABLE 1-1A
Hybrid, WHcAg-RSV VLPs Descriptions\hat{ }

| VLP No. | SEQ ID NO. | Insert | Position | Carrier Δ | Epitope Δ | Linker Δ |
|---|---|---|---|---|---|---|
| VLP018 | 7 | RSV1 | 78 | C-trunc. | — | − |
| VLP019 | 8 | RSV1 | 78 | — | — | − |
| VLP020 | 9 | RSV2 | N-term | — | 34-mer | + |
| VLP021 | 10 | RSV2 | N-term | — | 34-mer | + |
| VLP021.1 | 11 | RSV2 | N-term | C-trunc. | 34-mer | + |
| VLP023 | 12 | RSV2 | 78 | — | 34-mer | + |
| VLP025 | 13 | RSV1 small | 65/84 | Δ66-83 | 34-mer | − |
| VLP027 | 14 | RSV3.1 | 78 | — | 39-mer | − |
| VLP028 | 15 | RSV4 | C-term | — | 63-mer | − |
| VLP029 | 16 | RSV4 | C-term | Δ6 | 63-mer | − |
| VLP030 | 17 | RSV4 | N-term | — | 63-mer | + |
| VLP031 | 18 | RSV4 | N-term | C-trunc. | 63-mer | + |
| VLP032 | 19 | RSV3.2 | 78 | — | 44-mer | − |
| VLP033 | 20 | RSV3.2 | 81 | — | 44-mer | − |
| VLP034 | 21 | RSV4 | C-term | Δ6.1 | 63-mer | − |
| VLP041 | 22 | RSV2 | 78 | C-trunc. | 34-mer | − |
| VLP042 | 23 | RSV2 | 81 | — | 34-mer | − |
| VLP043 | 24 | RSV2 | 78 | Δ2 | 34-mer | − |
| VLP044 | 25 | RSV2 | 78 | — | 35-mer | − |
| VLP045 | 26 | RSV1 | N-term | — | — | + |
| VLP046 | 27 | RSV1 | N-term | C-trunc. | — | + |
| VLP047 | 28 | RSV1 | 74 | — | — | + |
| VLP048 | 29 | RSV1 | 81 | — | — | − |
| VLP049 | 30 | RSV1 | 78 | Δ2 | — | − |
| VLP050 | 31 | RSV2 | 81 | — | 34-mer | + |
| VLP051 | 32 | RSV2 | 78 | C-trunc. | 34-mer | + |
| VLP052 | 33 | RSV2 | 78 | Δ2 | 34-mer | + |
| VLP053 | 34 | RSV2 | 78 | — | 34-mer | + |
| VLP059 | 35 | RSV1B | 78fuse | — | — | + |
| VLP060 | 36 | RSV5 | 78fuse | — | 21-mer | + |
| VLP061 | 37 | RSV1 | 74 | Δ7 | — | + |
| VLP062 | 38 | RSV1 | 74 | Δ7.1 | — | + |
| VLP063 | 39 | RSV2 | 78 | — | 34-mer | + |
| VLP064 | 40 | RSV6 | 78 | — | Loop | + |
| VLP068 | 41 | RSV7 | 78 | — | Loop | + |
| VLP069 | 42 | RSV8 | 78 | — | Loop | + |
| VLP072 | 43 | RSV1 | 76 | — | — | − |
| VLP073 | 44 | RSV1 | 82 | — | — | − |
| VLP074 | 45 | RSV1A | 78 | — | — | + |
| VLP075 | 46 | RSV1B | 78 | — | — | + |
| VLP076 | 47 | RSV1C | 78 | — | — | + |
| VLP077 | 48 | RSV1 scram2 | 78 | — | scramble | − |
| VLP078 | 49 | RSV1 | 78 | Δ6 | — | − |
| VLP079 | 50 | RSV1 | 78 | Δ6.1 | — | − |
| VLP080 | 51 | RSV1 | 78 | C-trunc. | — | − |
| VLP081 | 52 | RSV1 | 78 | C-trunc. | — | − |
| VLP087 | 53 | RSV1 | 78 | C-trunc. | — | − |
| VLP088 | 54 | RSV1 | 78 | Δ3 | — | − |
| VLP089 | 55 | RSV9 | 78 | — | — | + |
| VLP090 | 56 | RSV10 | 78 | — | N262H | − |
| VLP091 | 57 | RSV11 | 78 | — | Loop | + |
| VLP092 | 58 | RSV12 | 78 | — | Loop | + |
| VLP093 | 59 | RSV1 | 78 | Δ4 | — | − |
| VLP094 | 60 | RSV13 | 78fuse | — | +1N | + |
| VLP095 | 61 | RSV14 | 78fuse | — | +2N | + |
| VLP096 | 62 | RSV15 | 78fuse | — | +3N | + |
| VLP097 | 63 | RSV16 | 78fuse | — | +1C | + |
| VLP098 | 64 | RSV17 | 78fuse | — | +2C | + |
| VLP099 | 65 | RSV18 | 78fuse | — | +3C | + |
| VLP111 | 66 | RSV1B | 74/80fuse | Δ75-79 | — | + |
| VLP112 | 67 | RSV13 | 74/80fuse | Δ75-79 | +1N | + |
| VLP113 | 68 | RSV16 | 74/80fuse | Δ75-79 | +1C | + |
| VLP114 | 69 | RSV19 | 74/80fuse | Δ75-79 | +1N +1C | + |
| VLP120 | 70 | RSV1B | 61/86 | Δ5 | — | + |
| VLP121 | 71 | RSV1B | 62/87 | Δ5 | — | + |
| VLP122 | 72 | RSV1B | 6388 | Δ5 | — | + |
| VLP123 | 73 | RSV1B | 64/89 | Δ5 | — | + |
| VLP124 | 74 | RSV5 | 63/87 | Δ5 | 21mer | + |
| VLP125 | 75 | RSV1B | 61/86 | Δ4/Δ5 | — | + |
| VLP126 | 76 | RSV1B | 62/87 | Δ4/Δ5 | — | + |
| VLP127 | 77 | RSV1B | 63/88 | Δ4/Δ5 | — | + |
| VLP128 | 78 | RSV1B | 64/89 | Δ4/Δ5 | — | + |
| VLP129 | 79 | RSV5 | 63/87 | Δ4/Δ5 | 21-mer | + |
| VLP130 | 80 | RSV1B | 78 | — | — | + |
| VLP131 | 81 | RSV1B | 78 | — | — | + |
| VLP132 | 82 | RSV1B | 78 | — | — | + |
| VLP133 | 83 | RSV1B | 78 | — | — | + |
| VLP134 | 84 | RSV1B | 78 | — | — | + |
| VLP135 | 85 | RSV1B | 78 | — | — | + |

\hat{ }The carrier, epitope and linker modifications are described in comparison with a standard hybrid WHcAg: GILE - hAg - L inserted within the immunodominant loop (residues 76 to 82) of a full length WHcAg.
(—) indicates that no changes were made with reference to the standard, hybrid WHcAg.

TABLE 1-1B
RSV F Protein Inserts With or Without Linkers\hat{ }

| | | | | SEQ |
|---|---|---|---|---|
| | N-term. | | C-term. | ID |
| Name | linker | RSV F Sequence | linker | NO |
| RSVA | | NSELLSLINDMPITNDQKKLMSNN | | 3 |
| RSVB | | NSELLSLINDMPITNDQKKLMSSN | | 86 |
| MARM S275F | | NSELLSLINDMPITNDQKKLMFNN | | 87 |
| RSV1 | GILE | NSELLSLINDMPITNDQKKLMSNN | L | 88 |
| RSV1A | (GILE)A | NSELLSLINDMPITNDQKKLMSNN | L | 89 |
| RSV1B | (GILE) | NSELLSLINDMPITNDQKKLMSNN | L | 90 |
| RSV1C | GILEE | NSELLSLINDMPITNDQKKLMSNN | EEL | 91 |
| RSV1scram2 | GILE | IKQSLMTDSLSNPNNLNNDIKLEM | L | 92 |
| RSV1 small | (GILE) | SELLSLINDMPITNDQKKLMS | (L) | 93 |
| RSV2 | GIL(E) | TYMLTNSELLSLINDMPITNDQKKLMSNNVQIVR | L | 94 |
| RSV3.1 | GIL(E) | VNAGVTTPVS | L | 95 |
| | | TYMLTNSELLSLINDMPITNDQKKLMSNN | | |
| RSV3.2 | GIL(E) | VNAGVTTPVS | L | 96 |
| | | TYMLTNSELLSLINDMPITNDQKKLMSNNVQIVR | | |
| RSV4 | GIL(E) | SNIETVIEFQQKNNRLLEITREFSVNAGVTTPVS | L | 97 |
| | | TYMLTNSELLSLINDMPITNDQKKLMSNN | | |
| RSV5 | (GILE) | ELLSLINDMPITNDQKKLMSN | (L) | 98 |
| RSV6 | GIL(E) | NSELLSLINDLPASNDQKKLMSNN | L | 99 |
| RSV7 | GIL(E) | NSELLSLINDAPAANDQKKLMSNN | L | 100 |
| RSV8 | GIL(E) | NSELLSLINDAAAANDQKKLMSNN | L | 101 |
| RSV9 | GILPE | NSELLSLINDMPITNDQKKLMSNN | PEL | 102 |
| RSV10 | GILE | NSELLSLIHDMPITNDQKKLMSNN | L | 103 |
| RSV11 | GIL(E) | NSELLSLINDMPAANDQKKLMSNN | L | 104 |
| RSV12 | GIL(E) | NSELLSLINDAPASNDQKKLMSNN | L | 105 |
| RSV13 | (GILE) | TNSELLSLINDMPITNDQKKLMSNN | (L) | 106 |
| RSV14 | (GILE) | LTNSELLSLINDMPITNDQKKLMSNN | (L) | 107 |
| RSV15 | (GILE) | MLTNSELLSLINDMPITNDQKKLMSNN | (L) | 108 |
| RSV16 | (GILE) | NSELLSLINDMPITNDQKKLMSNNV | (L) | 109 |
| RSV17 | (GILE) | NSELLSLINDMPITNDQKKLMSNNVQ | (L) | 110 |
| RSV18 | (GILE) | NSELLSLINDMPITNDQKKLMSNNVQI | (L) | 110 |
| RSV19 | (GILE) | TNSELLSLINDMPITNDQKKLMSNNV | (L) | 111 |
| MARM S275L | | NSELLSLINDMPITNDQKKLMLNN | | 112 |
| MARM K272E | | NSELLSLINDMPITNDQKELMFNN | | 113 |
| MARM K272Q | | NSELLSLINDMPITNDQKQLMSNN | | 114 |

\hat{ }Standard linker combination is GILE-Xn-L, where X is any amino acid, and n is 60 or less (SEQ ID NO: 5). Deleted residues are shown within parenthesis ( ), and added residues are shown in bold.

The Majority of the Hybrid, WHcAg-RSV VLPs Assembled Efficiently.

Of the hybrid, WHcAg-RSV VLPs attempted, 70% were expressed and assembled efficiently due in large part to various modifications. A summary of the characteristics of the hybrid, WHcAg-RSV VLPs designed and tested during development of the present disclosure is provided as Table 1-1C.

TABLE 1-1C
Hybrid, WHcAg-RSV VLPs Characteristics\hat{ }

| | VLP | MAb | anti-RSV-F | Neut | |
|---|---|---|---|---|---|
| VLP No. | Assembly | Binding | IgG Titer | Titer | Protection |
| VLP018 | + | + | Hi | Int | Int |
| VLP019 | + | + | Hi | Hi | Hi/Int |
| VLP020 | − | na | na | na | na |
| VLP021 | − | na | na | na | na |
| VLP021.1 | − | na | na | na | na |
| VLP023 | + | + | Hi | 0 | nd |
| VLP025 | − | na | na | na | na |
| VLP027 | + | + | Hi | 0 | nd |
| VLP028 | − | na | na | na | na |
| VLP029 | − | na | na | na | na |
| VLP030 | − | na | na | na | na |
| VLP031 | − | na | na | na | na |
| VLP032 | − | na | na | na | na |
| VLP033 | + | + | Hi | Int | nd |
| VLP034 | − | na | na | na | na |
| VLP041 | − | na | na | na | na |
| VLP042 | − | na | na | na | na |
| VLP043 | − | na | na | na | na |
| VLP044 | − | na | na | na | na |
| VLP045 | + | + | Hi | 0 | 0 |
| VLP046 | + | + | Hi | 0 | 0 |
| VLP047 | − | na | na | na | na |
| VLP048 | + | + | Hi | nd | 0 |
| VLP049 | + | + | Hi | Int | Hi |
| VLP050 | + | + | Hi | Low | Int |
| VLP051 | − | na | na | na | na |
| VLP052 | + | + | Hi | Low | Int |
| VLP053 | + | + | nd | nd | nd |
| VLP059 | + | + | Hi | Int | Int |
| VLP060 | + | + | Hi | Int | Int |
| VLP061 | + | + | Hi | nd | nd |
| VLP062 | + | + | Hi | Low | Int |
| VLP063 | + | + | Hi | 0 | 0 |
| VLP064 | + | + | Hi | 0 | 0 |
| VLP068 | + | + | Hi | 0 | 0 |
| VLP069 | − | na | na | na | na |
| VLP072 | + | + | Hi | nd | Low |
| VLP073 | + | 0 | Hi | nd | 0 |
| VLP074 | + | + | Hi | Int | Int |
| VLP075 | + | + | Hi | Hi | Hi |
| VLP076 | + | + | Hi | 0 | 0 |
| VLP077 | + | 0 | 0 | 0 | 0 |
| VLP078 | + | + | Hi | Int | Int |
| VLP079 | − | na | na | na | na |
| VLP080 | + | + | Hi | Int | Hi |
| VLP081 | − | na | na | na | na |
| VLP087 | + | + | Hi | Hi | Hi |
| VLP088 | + | + | Hi | Int | Int |
| VLP089 | + | + | Hi | 0 | Low |
| VLP090 | + | + | Hi | Low | Hi |
| VLP091 | + | + | Hi | Low | Low |
| VLP092 | + | + | Hi | 0 | 0 |
| VLP093 | + | + | Hi | Hi | Hi (mouse) |
| VLP094 | + | + | Hi | 0 | 0 |
| VLP095 | + | + | Hi | 0 | 0 |
| VLP096 | + | + | Hi | Low | Int |
| VLP097 | + | + | Hi | Hi | Int |
| VLP098 | + | + | Hi | 0 | Low |
| VLP099 | + | + | Hi | 0 | 0 |
| VLP111 | + | + | Hi | 0 | 0 |
| VLP112 | + | + | Hi | 0 | 0 |
| VLP113 | + | + | Hi | 0 | Low |
| VLP114 | − | na | na | na | na |
| VLP120 | + | + | Hi | 0 | nd |
| VLP121 | − | na | na | na | na |
| VLP122 | − | na | na | na | na |
| VLP123 | + | + | Hi | Hi | nd |
| VLP124 | + | + | Hi | 0 | nd |
| VLP125 | + | + | Hi | 0 | nd |
| VLP126 | − | na | na | na | na |
| VLP127 | − | na | na | na | na |
| VLP128 | + | + | Hi | Hi | nd |
| VLP129 | + | + | Hi | 0 | nd |
| VLP130 | + | + | Hi | Int | nd |
| VLP131 | + | + | Hi | Hi | nd |
| VLP132 | + | + | Hi | Hi | nd |
| VLP133 | + | + | Hi | Low | nd |
| VLP134 | + | + | Hi | Low | nd |
| VLP135 | + | + | Hi | Hi | nd |

\hat{ }Legend: na, not applicable, and nd, not done.
VLP Assembly: (+) sufficient assembly; or (−) insufficient assembly.
anti-RSV-F IgG Titers (log2): Hi ≧11, Int 8-10, Low ≦7, or 0 = not detectable;
Neutralization Titers (log2): Hi >7, Int 5-7, Low 4-5, or 0 < 4; and
Protection (log10 reduction in RSV titer): Hi >2.0, Int 1.0-2.0, Low 0.5-1.0, and 0 < 0.5.

Antigenicity of hybrid VLPs for palivizumab binding.

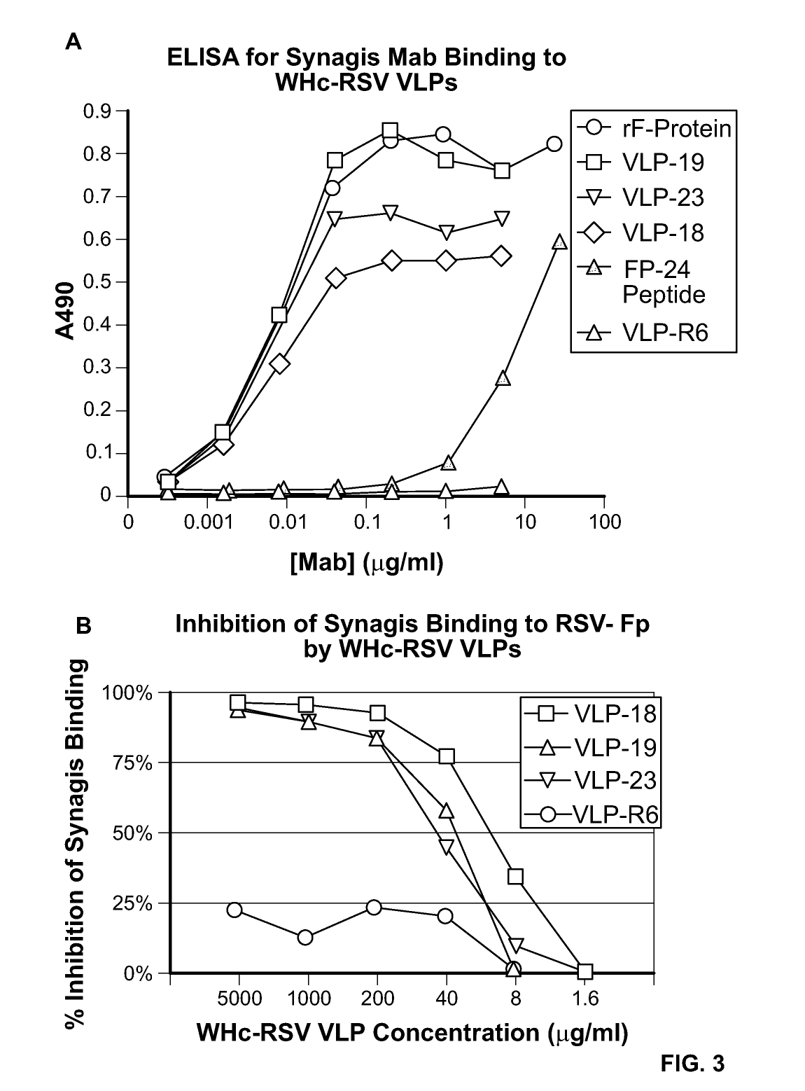

Purified hybrid VLPs were tested for antigenicity for palivizumab by two methods. Palivizumab bound virtually all of the solid-phase hybrid VLPs, albeit with different efficiencies as shown in FIG. 3A. Palivizumab bound VLP-19 as well as the intact RSV recombinant F (rF) protein. However, palivizumab bound the F²⁵⁴⁻²⁷⁷ 24aa peptide very poorly as compared to the rF protein and the hybrid VLPs containing the 24aa insert, indicating the necessity of correct palivizumab epitope conformation. This assay also demonstrated the ability of the hybrid VLPs to present the RSV B cell epitope correctly. The correct conformation is also indicated by the fact that IgG in human plasma from RSV-exposed humans binds rF protein and VLP019 similarly (Table 1-2). The ability of the hybrid, WHcAg-RSV VLPs to mimic the palivizumab epitope from the RSV rF protein with a binding efficiency similar to the native protein indicates that these VLPs could be exploited for use in measuring palivizumab-like antibody responses of naturally infected individuals, as well as those who have received vaccines including the RSV F protein.

TABLE 1-2
Recognition of rF Protein and Hybrid,
WHcAg-RSV VLPs by Human Plasma

ELISA Endpoint Titers (log2)

| | Young Adult | Elderly |
|---|---|---|
| | (20-30 years) | (65-85 years) |
| Antigen/Plasma | n = 19 | n = 23 |
| RSV rF protein | 12.8 ± 1.5 | 12.7 ± 2.2 |
| VLP019 | 11.4 ± 1.1 | 11.1 ± 1.4 |

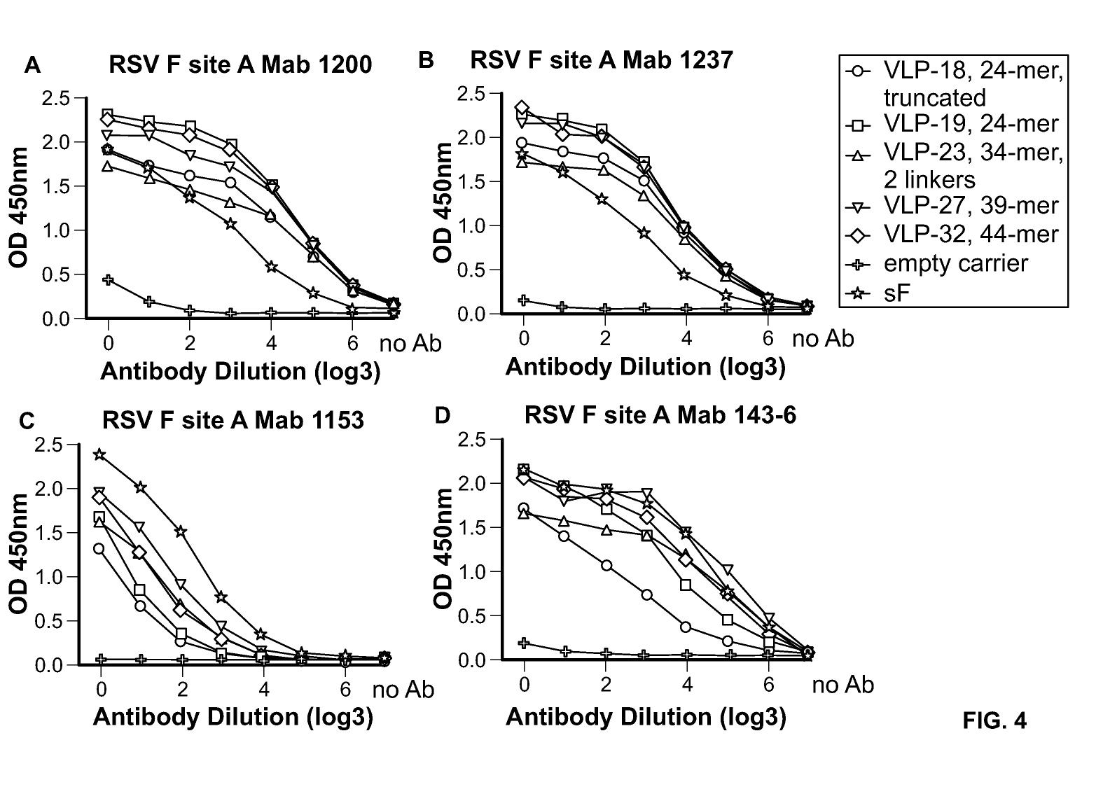

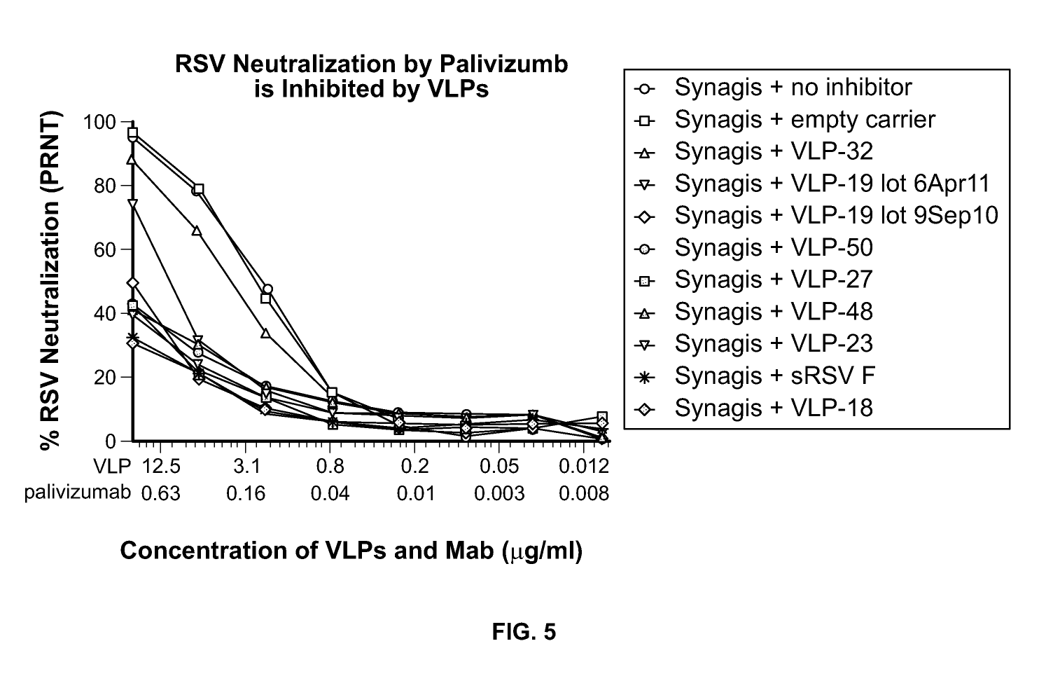

Hybrid VLPs in solution also efficiently bound palivizumab and inhibited palivizumab from binding solid-phase rF protein at relatively low concentrations of hybrid VLPs (50% inhibition at between 8-40 ng/ml) as shown in FIG. 3B. Several other RSV MAbs bound the solid-phase hybrid VLPs as shown in FIG. 4A-D. A number of other hybrid WHcAg-RSV VLPs are also capable of inhibiting the RSV neutralization activity of palivizumab as shown in FIG. 5.

Immunogenicity of Hybrid VLPs in Mice.

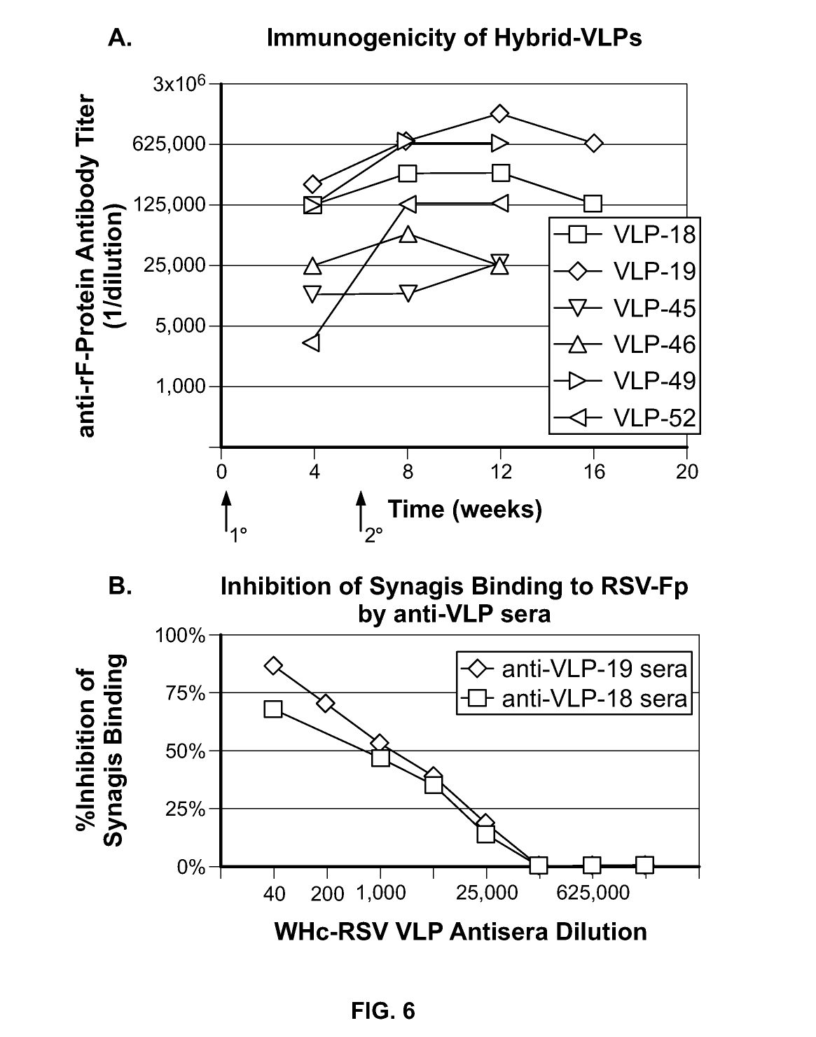

All hybrid VLPs were immunogenic in mice and immunization with 20 μg of the VLPs in incomplete Freunds adjuvant (IFA) elicited varying levels of anti-F²⁵⁴⁻²⁷⁷ antibodies that bound the 24aa peptide but more importantly bound the intact rF protein with end-point dilution titers between 2.5×10⁴ and 1.2×10⁶ after a single boost with 10 μg of the VLPs as shown in FIG. 6A. The anti-F antibodies were composed of equal or greater proportions of IgG2a versus IgG1. To measure fine specificity of the anti-VLP antisera for the palivizumab epitope on rF protein, anti-VLP antisera was tested for its ability to block palivizumab binding to solid phase rF protein. As shown in FIG. 6B, the ability of anti-VLP-18 and anti-VLP-19 antisera to inhibit palivizumab-binding 50% at dilutions of 1:1000 indicate that the anti-hybrid VLP antisera contained palivizumab-like antibodies that could compete with palivizumab for binding to the intact rF protein. Antisera to most hybrid-VLPs demonstrated inhibition of palivizumab binding to rF-protein to varying degrees. VLP-19, which contains RSV F254-277 inserted at residue 78 of full-length WHcAg VLP and encapsidates ssRNA (TLR7 ligand), was shown to elicit a high level of protection against RSV infection as described below.

WHcAg VLP Displays RSV F Aa254-277 on its Surface.

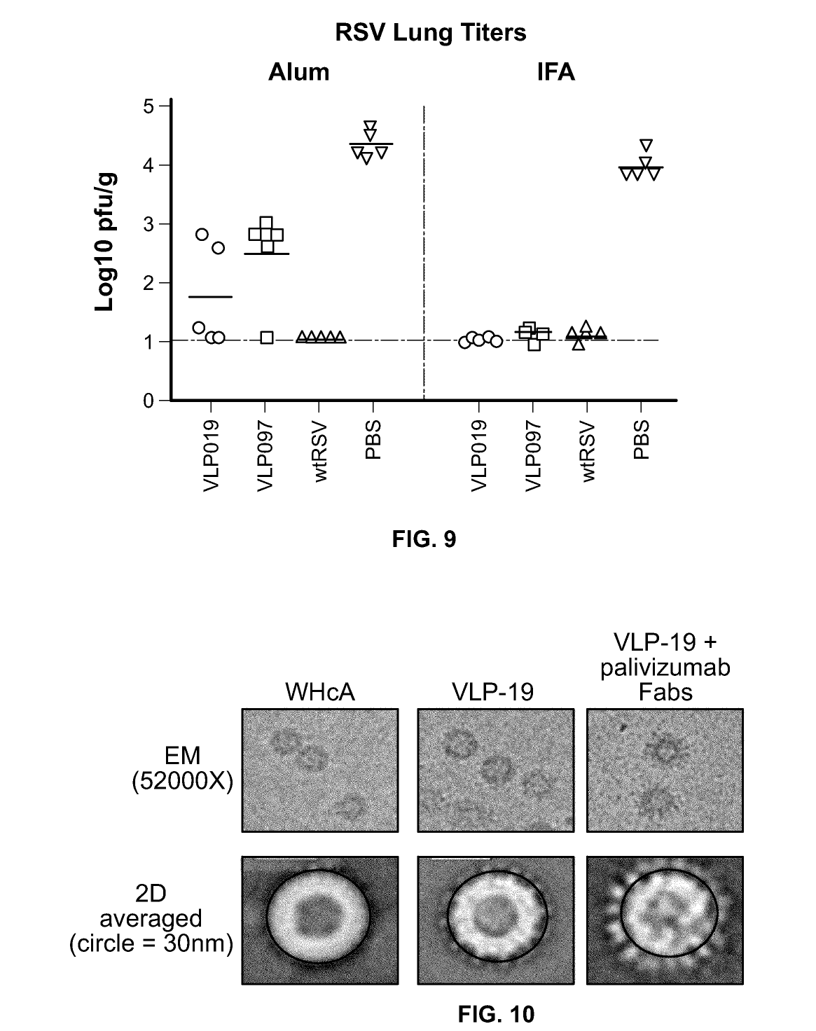

Cryoelectron microscopic analysis was performed to characterize VLP-19 visually. At 52,000× magnification, the particles carrying the insertions had a rougher surface appearance compared to the empty carrier WHcAg particles (FIG. 10). Averaging performed by Nanoimaging Services (La Jolla, Calif.) revealed that the surface was uniformly covered with spikes extending 2-4 nm from the surface of the spherical, approximately 29 nm diameter particles. To determine whether the spikes indeed contained the 24-mer insert in the appropriate conformation, palivizumab Fabs were bound to the VLPs and electron microscopic analysis analysis was again performed. The resulting images (FIG. 10) are consistent with palivizumab Fabs binding to VLP surface spikes. These results demonstrate that the 24-mer encompassing aa254-277 of the RSV F protein was successfully expressed on the surface of fully-formed WHcAg VLPs.

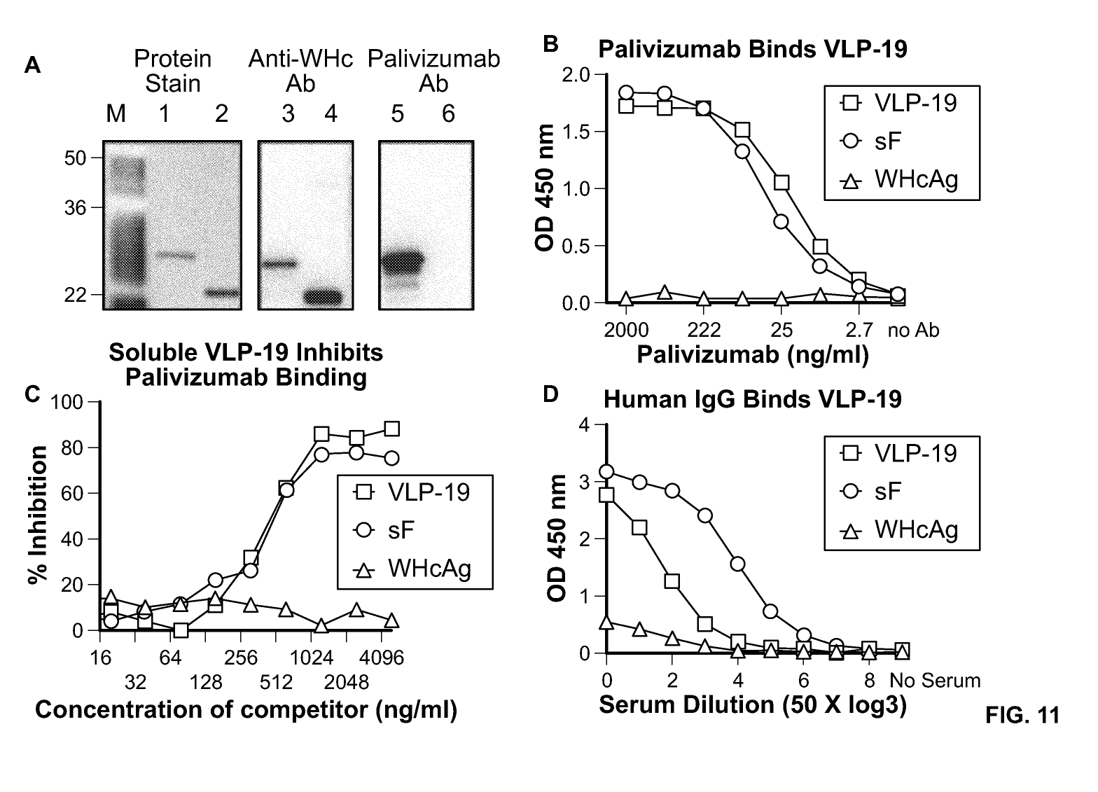

SDS PAGE and Western blot analysis was performed on VLP-19 and the carrier WHcAg. Staining of the SDS PAGE gel to visualize proteins showed major bands at about 25 kD and about 22 kD for the monomers of VLP-19 and empty WHcAg carrier, respectively demonstrating that the monomer of VLP-19 contains an insert of the expected size (FIG. 11A, lanes 1 and 2). Following transfer to a PVDF membrane, both VLP-19 and WHcAg were recognized by rabbit polyclonal antiserum against WHcAg (FIG. 11A, lanes 3 and 4), while only VLP-19 was recognized by palivizumab (FIG. 11A, lanes 5 and 6).

The ability of palivizumab to recognize VLP-19 in solid phase bound to an ELISA plate and in solution with a competitive ELISA assay was tested. In both the direct ELISA and competitive ELISA formats, the palivizumab binding curves for VLP-19 were comparable to those obtained for purified recombinant soluble RSV F (sF) (FIG. 11B and FIG. 11C). Taken together, these data indicate that the antigenicity of the palivizumab-specific RSV F254-277 epitope is maintained in the context of VLP-19.

Human IgG Recognizes VLP-19.

To determine whether the RSV F aa254-277 epitope displayed on VLP-19 is antigenically related to the epitope present during natural RSV infection, human plasma was tested for antibody specific for VLP-19 by ELISA. Because nearly all people are seropositive for RSV by age two and re-exposed several times throughout life, normal human plasma contains RSV antibodies (Walsh and Falsey, J Med Virol, 73:295-299, 2004). Human IgG in each of 42 adult plasma samples bound efficiently to VLP-19 (Table 1-2, and FIG. 11D). Though sF has a greater variety of F-specific epitopes than VLP-19, the endpoint titer for sF was less than two log 2 higher when compared to VLP-19 in ELISA. Thus, human IgG raised in response to RSV infection efficiently recognizes the F254-277 epitope as expressed on VLP-19.

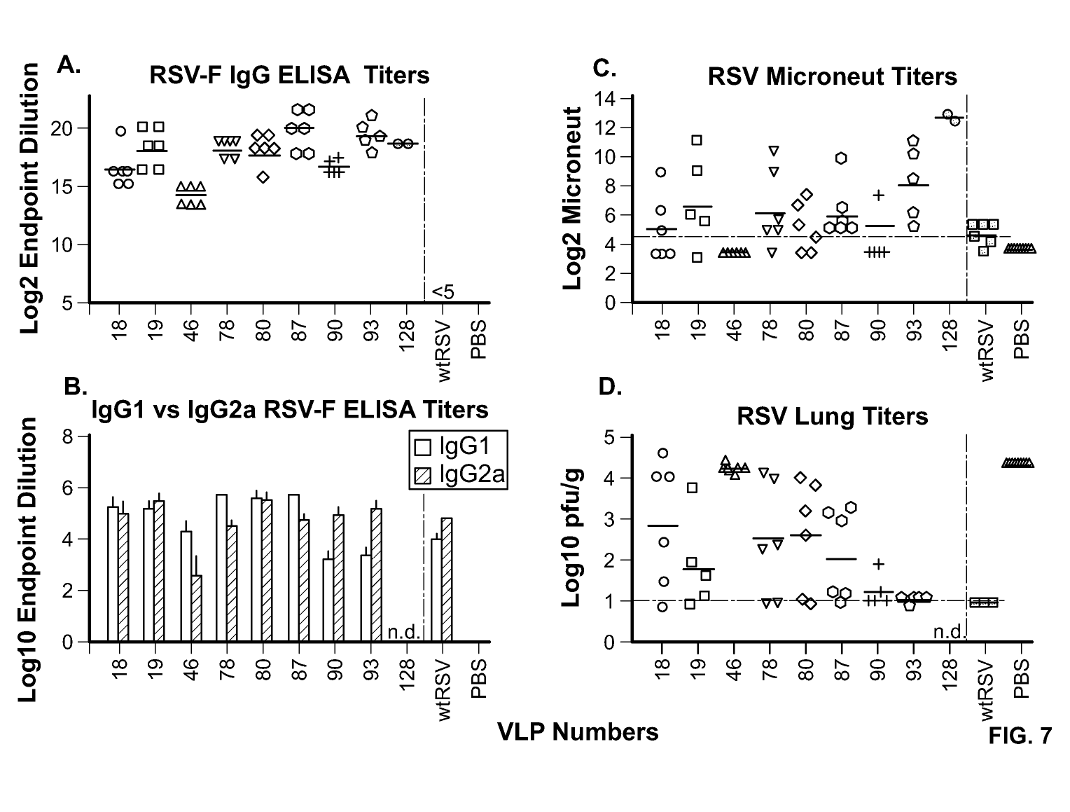

Ability of hybrid VLPs to elicit RSV neutralizing antibodies. Although all assembled hybrid VLPs elicited high titer IgG anti-rF protein antibodies, hybrid VLPs varied dramatically in ability to elicit high titer, RSV-specific neutralizing antibodies as shown in FIG. 7A-FIG. 7C. In this experiment mice were immunized and boosted once with 100 μg of the hybrid VLPs in an alum formulation. After the boost, sera were tested by ELISA for IgG binding to rF protein (FIG. 7A), IgG isotype distribution of anti-rF antibodies as a measure of Th2 and Th1 like antibodies (FIG. 7B), and in a RSV plaque reduction microneutralization assay to measure neutralizing antibodies (FIG. 7C).

Most hybrid VLPs elicited significant neutralizing antibodies in only half or fewer of mice of each group (FIG. 7C) despite high levels of IgG, rF protein-binding antibodies in all mice/group (FIG. 7A). All hybrid VLPs elicited a relatively balanced Th1/Th2-like response (FIG. 7B). The ratio of [IgG anti-F protein]/[neutralizing anti-RSV] antibodies was quite high in most anti-VLP antisera, indicating that functional neutralizing antibodies can represent a minority of the anti-insert response. However, hybrid VLPs like VLP-93 represent an exception, in that this hybrid VLP is able to elicit high neutralizing, as well as high IgG-binding antibodies in 100% of injected mice. This illustrates the importance of screening for neutralizing activity, as well as IgG-binding to rF protein by ELISA when selecting a VLP as an immunogen. Summaries of hybrid VLP immunological profiles are provided in Table 1-3 and Table 1-4 (̂Legend: nd, not done. Mean Neutralization Titers (log 2): Hi>7, Int 5-7, Low 4-5, or 0=below limit of detection; Mean Protection (log 10 reduction in RSV lung titer): Hi>2.0, Int 1.0-2.0, Low 0.5-1.0, or nd=not done; and *=VLPs that do not bind TLR7 ligands).

TABLE 1-3
VLPS Eliciting High Neutralizing Ab Titers And/Or
Protection From RSV Challenge\hat{ }

| | Neut | | |
|---|---|---|---|
| VLP | Titer | Protection | Characteristics |
| VLP019 | Hi | Hi | Standard: 24-mer insert at position 78 |
| VLP049 | Int | Hi | Standard with Δ2 |
| VLP075 | Hi | Hi | Standard with (E) deleted from linker |
| VLP080 | Int | Hi | Standard with WHcAg C-terminus |
| | | | mutated after 149 |
| VLP087 * | Int | Hi | Standard with WHcAg C-terminus |
| | | | mutated after 152 |
| VLP090 | Low | Hi | Standard with N262H RSV epitope |
| | | | point mutation |
| VLP093 | Hi | Hi | Standard with Δ4 |
| VLP097 | Hi | Int | Standard with no linkers and valine |
| | | | at C-termimus of RSV |
| VLP123 | Hi | nd | Δ5 |
| VLP128 | Hi | nd | Δ5 and Δ4 |
| VLP131 | Hi | nd | Standard with G_L linker only |
| VLP132 | Hi | nd | Standard with I_L linker only |
| VLP135 | Hi | nd | Standard with GIL_V linker only |

TABLE 1-4
VLPS Eliciting Intermediate-to-Low Neutralizing Ab Titers
And/Or Protection From RSV Challenge\hat{ }

| | Neut | | |
|---|---|---|---|
| VLP | Titer | Protection | Characteristics |
| VLP018 * | Int | Int | Standard with WHcAg C-terminus |
| | | | mutated after 149 |
| VLP050 | Low | Int | 34-mer insert with N-term 9aa linker |
| | | | at position 81 |
| VLP052 | Low | Int | 34-mer insert with C-term 9aa linker |
| | | | at position 78 of Δ2 |
| VLP059 | Int | Int | Standard with no linkers |
| VLP060 | Int | Int | Standard with 21-mer insert and no |
| | | | linkers |
| VLP062 | Low | Int | 24-mer insert at position 74 of Δ7.1 |
| VLP074 | Int | Int | Standard with (E)→A in linker |
| VLP078 | Int | Int | Standard with Δ6 |
| VLP088 | Int | Int | Standard with Δ3 |
| VLP091 | Low | Low | Standard with I266A/T267A RSV epitope |
| | | | point mutations |
| VLP096 | Low | Int | Standard w/o linkers and 3 extra |
| | | | F-protein residues |
| VLP098 | 0 | Low | Standard w/o linkers and 2 extra |
| | | | F-protein residues |
| VLP113 | 0 | Low | WHc Δ74-80 w/o linkers and 1 extra |
| | | | F-protein residue |
| VLP130 | Int | nd | Standard with GI_L linker only |
| VLP133 | Low | nd | Standard with IL_L linker only |
| VLP134 | Low | nd | Standard with L deleted from C-term |
| | | | linker |

Ability of Hybrid-VLPs to Protect Mice Against an RSV Challenge.

Immunized mice were also challenged with 10⁵ PFU of RSV two weeks after the final bleed. Recovery of RSV from homogenized lung was determined by plaque assay as a measure of the protective potential of hybrid VLP immunization in vivo (FIG. 7D). Reduction of RSV titers in the lung of two log 10 as compared to placebo are considered highly significant. The standard VLP-19 elicited a reduction of RSV lung titers of two log 10 in 4/5 mice, VLP-87 elicited complete protection (no RSV detected) in 50% of the mice and a reduction of two log 10 in the remaining 50% of the mice. VLP-90 elicited complete protection in 4/5 mice. VLP-93, which elicited the highest neutralizing antibody response with the exception of VLP-128, protected 100% of the mice (no virus detected) from RSV challenge, which is equal to the level of protection elicited by wild type RSV (FIG. 7D).

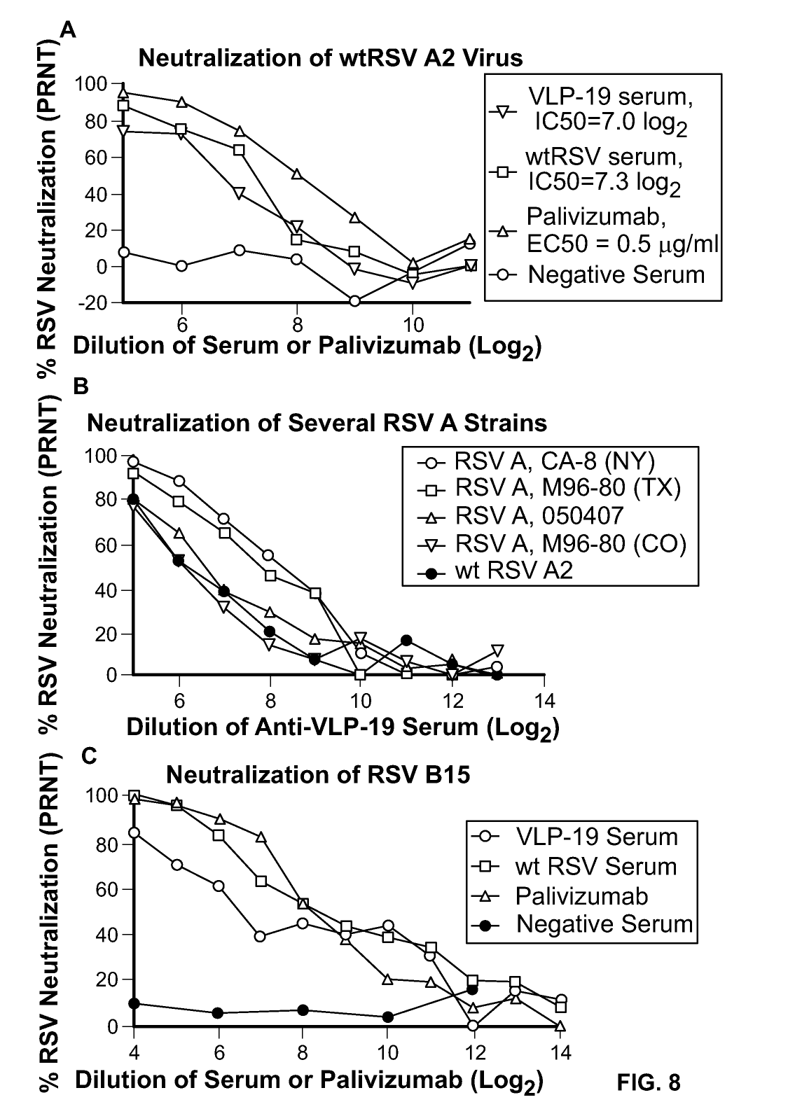

Additionally, VLP019 antiserum was found to neutralize RSV-A, RSV-B and a palivizumab escape mutant (MARM S275F). Dilutions of antiserum or palivizumab were mixed with 100-200 PFU of RSV virus (without complement), incubated for 1 hour and titers measured by plaque assay. FIG. 8A shows neutralization of RSV A2, FIG. 8B shows neutralization of several RSV A strains, FIG. 8C shows neutralization of RSV B15, FIG. 8D shows neutralization of RSV B77, FIG. 8E shows neutralization of the palivizumab escape mutant (MARM S275F), and FIG. 8F shows neutralization of additional escape mutants. Amino acid sequences of the RSV F proteins in the region of interest are as follows: RSV-A (SEQ ID NO: 3); RSV B (SEQ ID NO: 86); and RSV MARM S275F (SEQ ID NO: 87). These results are illustrative of the polyclonal nature of anti-VLP019 antisera. The use of two or more distinct hybrid, WHcAg-RSV VLPs should increase the diversity of the polyclonal response to an even greater extent.

Ability of VLP-19 to Protect Cotton Rats Against an RSV Challenge.

As shown in Table 1-5, four of seven cotton rats immunized with VLP-19 in alum had significant protection against an RSV challenge. Note that three of seven rats demonstrated the same level of protection (RSV titers<1.0 log 10) as rats immunized with the positive control RSV F protein. This is surprising given that the RSV F protein contains numerous neutralizing B cell epitopes, whereas VLP-19 is only known to include a single RSV F protein B cell epitope (e.g., palivizumab epitope). Also note that neutralization titers correlated with protection, but total anti-RSV F antibody titers did not. There was rat-to-rat variation in neutralization/protection, similar to the variation observed in mice immunized with various hybrid WHcAg-RSV VLPs. This is striking in that the anti-WHcAg, anti-RSV F protein and anti-RSV F peptide responses in cotton rats were not significantly variable.

TABLE 1-5
Antibody Titers and Level of Protection
From RSV Challenge in Rats

| | Anti- | Anti- | Anti- | Neut. | Lung |
|---|---|---|---|---|---|
| | WHcAg | RSV F | Peptide | Titer | Titer |
| VLP | (Log2) | (Log2) | (Log2) | (Log2) | (Log10) |
| VLP19 + alum | | | | | |
| 1 | 16 | 15 | 15 | 0 | 5.0 |
| 2 | 16 | 15 | 16 | 10 | 0.8 |
| 3 | 18 | 16 | 16 | 0 | 4.2 |
| 4 | 16 | 17 | 16 | 13 | 0.9 |
| 5 | 18 | 15 | 16 | 8 | 0.9 |
| 6 | 17 | 15 | 15 | 5 | 4.8 |
| 7 | 18 | 16 | 16 | 6 | 2.2 |
| Totals | 7/7 | 7/7 | 7/7 | 5/7 | 4/7* |
| RSV rF + alum | | | | | |
| 1 | 0 | 18 | 0 | 11 | 1.0 |
| 2 | 0 | 19 | 0 | 12 | 0.9 |
| 3 | 0 | 19 | 0 | 12 | 0.9 |
| Totals | 0/3 | 3/3 | 0/3 | 3/3 | 3/3* |
| PBS + alum | | | | | |
| 1 | 0 | 0 | 0 | 0 | 4.9 |
| 2 | 0 | 0 | 0 | 0 | 5.0 |
| 3 | 0 | 0 | 0 | 0 | 5.1 |
| Totals | 0/3 | 0/3 | 0/3 | 0/3 | 0/3 |

*signifies protection defined as reduction in RSV lung titer ≧ 2log10.

A small study was conducted in mice to determine whether individuals that were neutralizing antibody non-responders when dosed twice with a first hybrid VLP would become neutralizing antibody responders when dosed once with a second (different) hybrid VLP. As shown in Table 1-6, all non-responders became responders after receiving a single VLP019 boost. This is consistent with the same RSV 24-mer epitope assuming a different conformation in the context of different WHcAg carriers. Moreover, this observation indicates that combining two or more WHcAg-RSV VLPs is advantageous in circumventing inter-subject variation. This is an important concern when immunizing an outbred population of individuals (e.g., human subjects).

TABLE 1-6
A Single VLP019 Boost Elicits a Neutralizing Antibody Response

| | | Neut. Titer after |
|---|---|---|
| 1st and 2nd | Neut. Titer after | 3^{rd} (VLP019) |
| Immunization | 2^{nd} Immunization | Immunization |
| VLP074 | 0 | 10 |
| VLP078 #1 | 0 | 30 |
| VLP078 #2 | 10 | 90 |
| VLP080 | 10 | 30 |
| VLP087 | 0 | 26 |
| VLP088 | 0 | 35 |

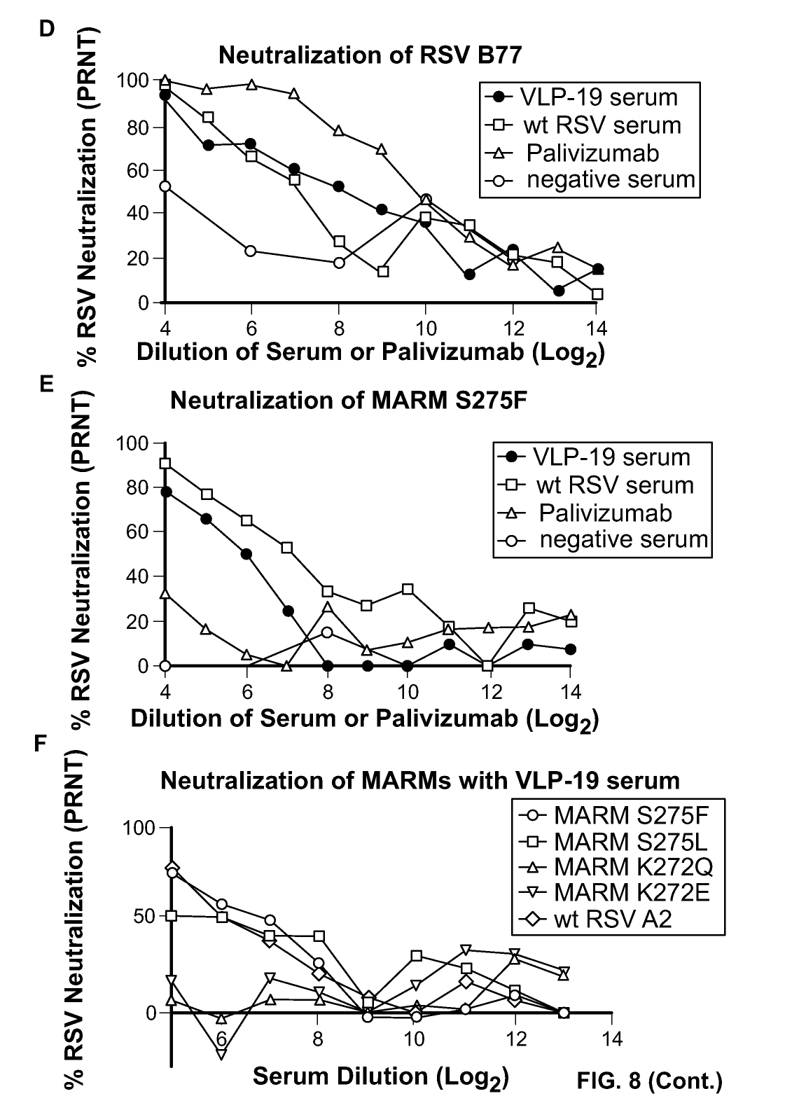

Another method to mitigate inter-subject variation in protective efficacy is to use an adjuvant stronger than alum for immunization. As shown in FIG. 9, 100% of the mice immunized with either VLP019 or VLP097 in IFA were completely protected against an RSV challenge. This level of protection is equal to the level of protection elicited by wild type RSV.

VLP-19 Elicits Protection.

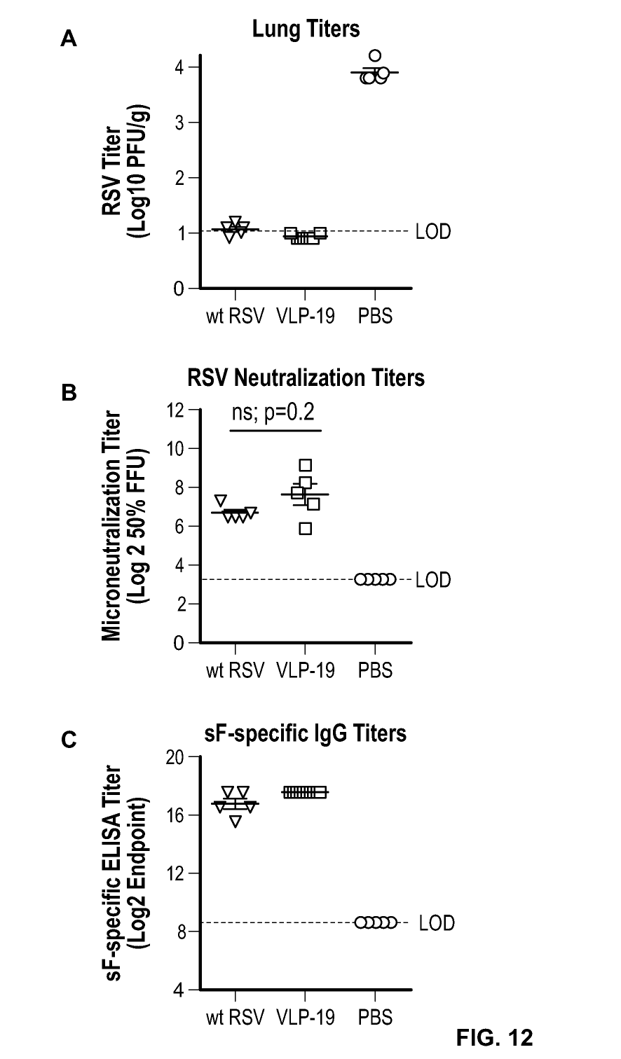

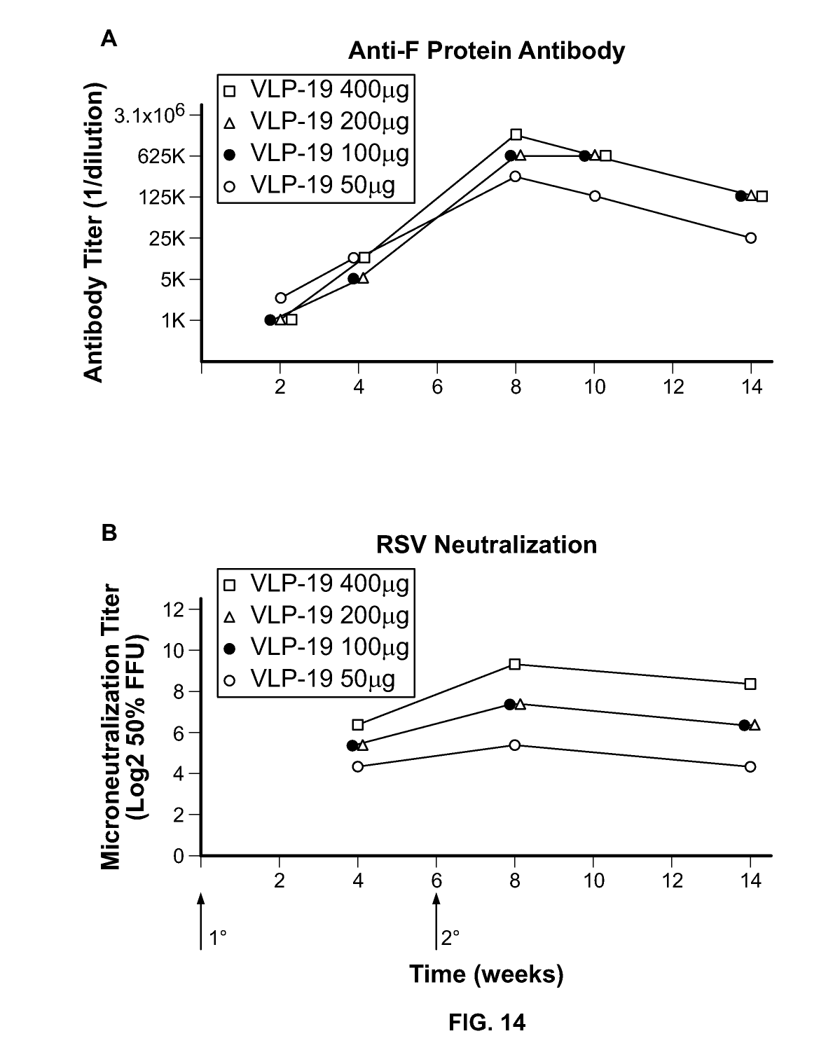

Balb/c mice were immunized with two doses of 40 μg VLP-19 formulated with incomplete Freund's adjuvant (IFA) and negative and positive control groups received either PBS alone or one intranasal administration of live wtRSVA2. Two weeks after the second dose, sera were analyzed for sF-specific IgG antibodies and for RSV neutralization and then mice were challenged with 10⁶ PFU wtRSVA2. Lung titers (log 10 PFU/g) of mice four days post challenge were 3.9+/−0.2 in the placebo group, and 1.1+/−0.1 and 0.9+/−0.1 for the wtRSV and VLP-19 groups, respectively (FIG. 12A). The RSV microneutralization titers in sera on the day of challenge were 6.7+/−0.4 and 7.7+/−1.2 log 2 for the wtRSV infected and VLP-19 immunized mice, respectively, which are not statistically different (p=0.2) (FIG. 12B). The sF-specific IgG titers were high for both groups, measuring 16.8+/−0.8 and 17.6+/−0.0 log 2 for the wtRSV infected and VLP-19 immunized groups, respectively (FIG. 12C). Thus, immunization of mice with two doses of VLP-19 was able to elicit a 1000-fold reduction in lung titer, and serum neutralizing and RSV F-specific IgG titers similar to mice following infection with wtRSV A2. While the exploratory work was performed with IFA, VLP-19 was injected in saline as well. Anti-F protein Ab and RSV neutralizing Ab titers elicited by VLP-19 were determined to be antigen dose-dependent, as opposed to adjuvant-dependent (FIG. 14A and FIG. 14B).

Anti-VLP-19 Sera is Broadly Neutralizing.

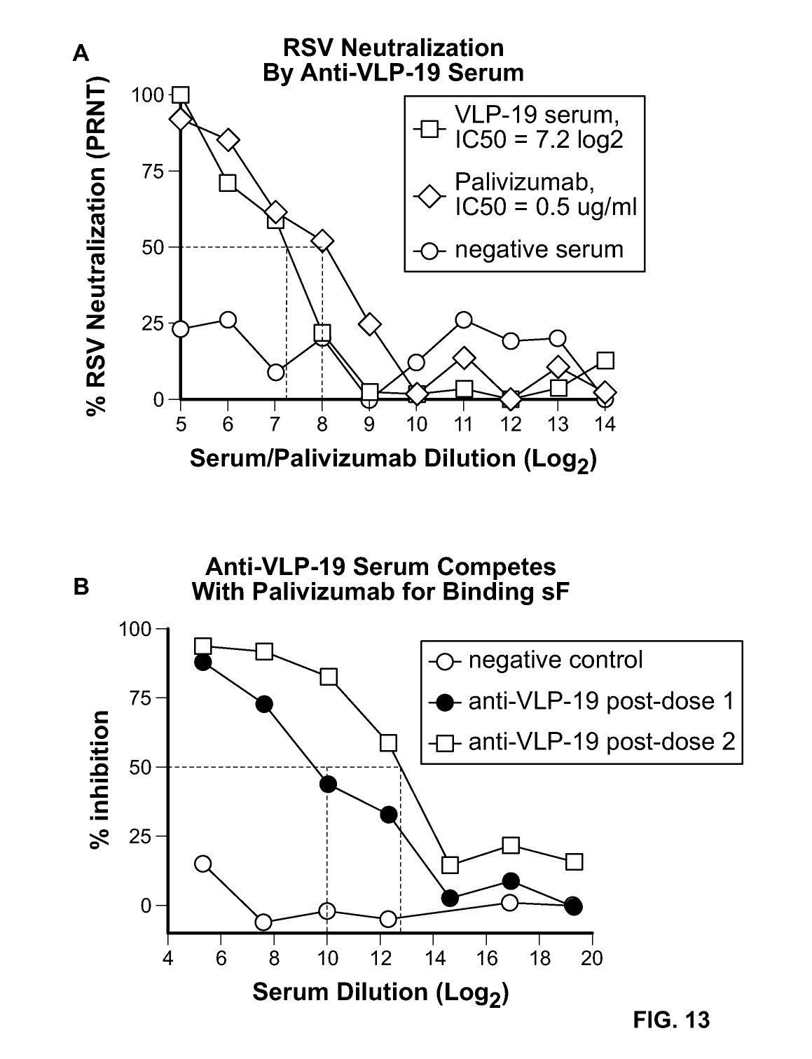

An RSV plaque reduction neutralization assay was performed with two-fold dilutions of anti-VLP-19 mouse sera and palivizumab. For anti-VLP-19 serum, the RSV neutralization titer (PRNT) as measured by the IC50 was 7.2 log 2, which corresponds to approximately an 1:150 dilution of sera (FIG. 13A). For palivizumab, the PRNT as measured by the IC50 point was at a concentration of about 0.5 μg/ml. Thus, anti-VLP-19 sera provided the equivalent neutralizing capability of about 75 μg/ml palivizumab in this in vitro assay.

The anti-VLP-19 sera were further evaluated and found to neutralize several RSV A and B clinical isolates, as well as the palivizumab antibody resistant mutants (MARMs) S275F and S275L (FIG. 8A-8F). However, anti-VLP-19 sera did not neutralize the K272Q or K272E MARMs. These results illustrate the polyclonal nature of the anti-VLP-19 response and differences in fine specificity as compared to palivizumab.

To investigate whether the anti-VLP-19 antibodies and palivizumab were directed to the same epitope on the RSV F protein, a competitive ELISA was performed. ELISA plates were coated with sF. Dilutions of sera from VLP-19 immunized mice or a negative control sera were mixed with a constant concentration of palivizumab, and allowed to bind to the sF-coated plates. Bound palivizumab was then measured. Anti-VLP-19 sera competed with palivizumab for binding to sF (FIG. 13B). The IC50 of the binding curves for anti-VLP-19 sera were ten log 2 and thirteen log 2 for post dose one and post dose two antisera, respectively, indicative of an increase in titer of palivizumab-competing antibodies following the boost.

Effect of the VLP-19 Insert Orientation.

The epitope RSV F254-277 has a helix-loop-helix motif and the contact points between the helices and motavizumab, an antibody that is derived from palivizumab, has been described (McLellan et al., Nat Struct Mol Biol, 17:248-250, 2010). This work suggests that the relative orientation of the two helices is critical for the correct presentation of the RSV F epitope. If the alpha helices of the VLP-19 insert are constrained in a favorable presentation, the amino acids between the insert and the VLP are predicted to affect the antibody response to the insert. The RSV F epitope was incrementally extended by up to three residues at the C-terminus and the resulting VLP constructs were tested for their ability to elicit a functional anti-RSV response. Three residues theoretically encompass roughly one revolution of an alpha helix.

VLP-19 has linker regions that flank the 24-mer insert to accommodate the restriction sites used to clone the target sequence into the WHcAg gene, as described. For this set of VLPs, the linker regions were first removed to juxtapose the alpha helices of the RSV F epitope more closely to those of the WHcAg. Then the inserted RSV F epitope was extended by one, two, or three amino acids on the C-terminus. The resulting VLPs were tested for palivizumab binding in vitro and protection and immunogenicity in vivo (Table 1-7). Removal of the short linker regions yielded similar RSV sF-specific IgG titers (VLP-59 vs. VLP-19), but reduced the ability of palivizumab to detect the VLP and reduced protection and neutralizing titers. Addition of one residue to the C-terminus of the insert (VLP-97) augmented the ability of palivizumab to detect the VLP and improved protection compared to VLP-59. However, addition of two residues to the C-terminus of the insert (VLP-98) reduced the ability of the VLP to be detected by palivizumab and reduced protection from challenge. The serum RSV neutralization and sF-specific IgG titers were also lower for VLP-98. Finally, addition of three residues (VLP-99) abolished the ability to elicit protection from challenge with wtRSV A2. Notably, although the ability of palivizumab to detect VLP-99 in vitro was a high (95%), VLP-99 was not able to protect mice from challenge with wtRSV A2. For VLP-99, palivizumab binding may be able to induce an in vitro conformation that the motif does not attain in vivo. VLP-99 was able to elicit sF-specific Ab in mice, but anti-VLP-99 sera did not neutralize RSV.

Taken together, these results indicate that the orientation of the alpha helices relative to each other in the helix-loop-helix motif is critical for the RSV F epitope to be displayed on the VLP in a manner that can elicit a protective immune response. These results also indicate that the epitope is subtly influenced by its specific insertion into the VLP, with small differences in presentation affecting both ability to be detected by palivizumab and the ability to elicit neutralizing antibody. Moreover, comparison of VLP-19, VLP-98 and VLP-99 demonstrate that palivizumab binding does not necessarily correlate with ability to elicit a RSV-neutralizing response.

TABLE 1-7
Characteristics of Modified VLPs.

| | | Palivizumab | RSV Lung Titer | | |
|---|---|---|---|---|---|
| | | binding | post challenge | | sF-specific |
| | | in vitro | (log10 +/− SD | RSV neut titer | IgG titer |
| Immunogen | Modification | (sF = 100%) | PFU/g) | (log2 +/− SD) | (log2 +/− SD) |
| Placebo | NA | NA | 3.9 +/− 0.2 | LOD* | LOD** |
| No Insert | | | | | |
| VLP-19 | linkers included | 100% | 0.9 +/− 0.1 | 7.7 +/− 1.2 | 17.2 +/− 0.5 |
| F 254-277 | | | | | |
| VLP-59 | linkers removed | 83% | 2.0 +/− 1.0 | 5.2 +/− 3.4 | 17.0 +/− 0.9 |
| F 254-277 | | | | | |
| VLP-97 | linkers removed | 112% | 1.1 +/− 0.1 | 6.9 +/− 2.8 | 17.2 +/− 0.5 |
| F 254-278 | (+1) | | | | |
| VLP-98 | linkers removed | 47% | 2.8 +/− 1.0 | 3.9 +/− 1.4 | 13.4 +/− 1.6 |
| F 254-279 | (+2) | | | | |
| VLP-99 | linkers removed | 95% | 3.8 +/− 0.3 | LOD* | 16.4 +/− 0.4 |
| F 254-280 | (+3) | | | | |

*LOD = 3.3 log2; and
**LOD = 8.6 log2.
This experiment was performed once with IFA (data shown) and once with a proprietary adjuvant that yielded similar results.

### CONCLUSIONS

A number of interesting observations were made. First, the total RSV IgG titer did not correlate with the RSV neutralizing antibody titer. While all VLPs elicited high titer anti-F protein IgG, only 24% of the hybrid VLPs elicited intermediate-to-high titer RSV neutralizing antibodies. Second, even amongst the VLPs that elicited RSV neutralizing antibodies, there was significant animal-to-animal variation, despite the use of inbred rodent strains. The ability of several hybrid VLPs (e.g., VLP093 and VLP090) to protect the majority (80-100%) of immunized animals against a RSV challenge indicates that the palivizumab epitope does adopt conformation resembling wild type RSV in a subset of hybrid WHcAg-RSV VLPs. This finding confirms the utility of screening a library of hybrid VLPs for identifying suitable immunogens.

Additionally, combinations of different hybrid VLPs, as well as different fusion proteins assembling into single mosaic VLPs are thought to be desirable in a RSV vaccine candidate. Furthermore, consolidation of modifications in a multiply-modified single VLP as in VLP0128 are also thought to be desirable for reducing non-responder frequencies. The VLP combinations and consolidations permit the production of antigenic compositions for eliciting a broad, functional antibody response with comparable anti-insert and anti-carrier antibody titers.

The approach to the design of an RSV vaccine described herein has been to display the epitope on a VLP such that the critical secondary structure is maintained. Immunization with VLP-19, encompassing aa254-277 displayed in an immunodominant region of the WHcAg VLP, provided 1000-fold reduction in lung titers in mice challenged with wt RSV A2 and elicited neutralizing antibody that competed with palivizumab for binding to RSV F. As determined during development of the present disclosure, an epitope can be antigenically correct (e.g., it can be recognized by antibody directed to F and elicit antibodies that recognize F), but nevertheless fail to generate neutralizing Abs (e.g., antibodies elicited to RSV F do not neutralize RSV). This is best illustrated by VLP-97 and -99, which differ only in encompassing RSV F aa254-278 or 254-280, respectively. While palivizumab bound both VLPs similarly, VLP-97 produced a potent RSV-neutralizing response and protected mice, but VLP-99 failed to elicit detectable RSV neutralization titers and provided no protection. This observation is consistent with a recent report that a correct RSV F254-278 structure did not always elicit a neutralizing Ab response, even when the F epitope scaffold elicited Abs that bound the immunogen (Correia et al., Nature, 2014 epub ahead of print, doi: 10.1038/nature12966).

Provided herein are functional analyses of the ability of selected chimeric VLPs to generate neutralizing antibodies and provide protection against RSV infection. This is believed to represent the first demonstration of potent neutralization and protection from RSV challenge provided by a recombinant epitope-focused immunogen displaying only RSV F254-277. This achievement also expands the application of the WHcAg-VLP technology to the presentation of epitopes that require a specific conformation. Generation of multiple protective VLPs illustrates the power of the WHcAg combinatorial technology. Further, preliminary evidence indicates that the neutralizing antibodies elicited by the various VLPs differ in fine specificities for the F254-277 epitope. Therefore, mixing multiple VLPs may be a means of increasing the diversity of neutralizing antibodies elicited by an epitope-based vaccine.

The WHcAg VLP may be uniquely suited as a platform for the F254-277 epitope in an RSV vaccine. The immunodominant spikes on the WHcAg are structurally similar to the F254-277 epitope in that both have a helix-loop-helix structure. The combinatorial technology developed for the WHcAg platform permits an empirical approach to reproduce the secondary structure of the F254-277 epitope on the VLP. The WHcAg VLP may also provide an advantage by reducing the potential for inducing enhanced respiratory disease (ERD) in naïve vaccinees. Non-neutralizing F-specific antibodies are implicated in ERD (Graham, Immunological Reviews, 239:149-166, 2011). Targeting a single neutralizing epitope removes the opportunity for production of non-neutralizing antibodies directed to non-site A epitopes of F protein. A Th1 bias and Toll-like receptor (TLR) 7 stimulation may also help to avoid ERD and contribute to production of protective antibody (Delgado et al., Nature Medicine, 15:34-41, 2008). WHcAg VLPs elicit Th1-biased antibody isotypes, which are enhanced by the adjuvant effect of encapsidated ssRNA that acts as a TLR7 agonist (Lee et al., J Immunol, 182:6670-6681, 2009); and Milich et al., J Virol, 71:2192-2201, 1997). In addition, WHcAg VLPs with the RSV F epitope displayed on the surface will not prime RSV F protein-specific T cells, which are implicated in enhanced respiratory disease (ERD)(Graham, supra, 2011). As a practical matter, WHcAg VLPs are inexpensive to produce, being fully recombinant, highly thermostable and expressable in bacteria, making the technology practical for use outside the first world. Thus, a WHcAg/RSV-F hybrid VLP approach offers the potential for the development of an RSV vaccine for the world.

### SEQUENCES

```
SEQ ID NO: 1
>full length WHcAg
MDIDPYKEFGSSYQLLNFLPLDFFPDLNALVDTATALYEEELTGREHCSPHHTAIRQALVCWDELTKLIAWMSSNIT
SEQVRTIIVNHVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
SEQ ID NO: 2
>truncated WHcAg
MDIDPYKEFGSSYQLLNFLPLDFFPDLNALVDTATALYEEELTGREHCSPHHTAIRQALVCWDELTKLIAWMSSNIT
SEQVRTIIVNHVNDTWGLKVRQSLWFHLSCLTFGQHTVQEFLVSFGVWIRTPAPYRPPNAPILSTLPEHTVI
SEQ ID NO: 3
>palivizumab epitope (RSV F)
NSELLSLINDMPITNDQKKLMSNN
SEQ ID NO: 4
>WHV genome
      1 aattcgggac ataccacgtg gtttagttcc gcctcaaact ccaacaaatc gagatcaagg
    61 gagaaagcct actcctccaa ctccacctct aagagatact cacccccact taactatgaa
  121 aaatcagact tttcatctcc aggggttcgt agacggatta cgagacttga caacaacgga
  181 acgccaacac aatgcctatg gagatccttt tacaacacta agccctgcgg ttcctactgt
  241 atccaccata ttgtctcctc cctcgacgac tggggaccct gcactgtcac cggagatgtc
  301 accatcaagt ctcctaggac tcctcgcagg attacaggtg gtgtatttct tgtggacaaa
  361 aatcctaaca atagctcaga atctagattg gtggtggact tctctcagtt ttccaggggg
  421 cataccagag tgcactggcc aaaattcgca gttccaaact tgcaaacact tgccaacctc
  481 ctgtccacca acttgcaatg gctttcgttg gatgtatctg cggcgtttta tcatatacct
  541 attagtcctg ctgctgtgcc tcatcttctt gttggttctc ctggactgga aaggtttaat
  601 acctgtctgt cctcttcaac ccacaacaga aacaacagtc aattgcagac aatgcacaat
  661 ctctgcacaa gacatgtata ctcctcctta ctgttgttgt ttaaaaccta cggcaggaaa
  721 ttgcacttgt tggcccatcc cttcatcatg ggctttagga aattacctat gggagtgggc
  781 cttagcccgt ttctcttggc tcaatttact agtgcccttg cttcaatggt taggaggaat
  841 ttccctcatt gcgtggtttt tgcttatatg gatgatttgg ttttgggggc ccgcacttct
  901 gagcatctta ccgccattta ttcccatatt tgttctgttt ttcttgattt gggtatacat
  961 ttgaatgtca ataaaacaaa atggtggggc aatcatctac atttcatggg atatgtgatt
1021 actagttcag gtgtattgcc acaagacaaa catgttaaga aaatttcccg ttatttgcgc
1081 tctgttcctg ttaatcaacc tctggattac aaaatttgtg aaagattgac tggtattctt
1141 aactatgttg ctccttttac gctatgtgga tacgctgctt taatgccttt gtatcatgct
1201 attacttccc gtacggcttt cattttctcc tccttgtata aatcctggtt gctgtctctt
1261 tatgaggagt tgtggcccgt tgtcaggcaa cgtggcgtgg tgtgcactgt gtttgctgac
1321 gcaaccccca ctggttgggg cattgccacc acctatcaac tcctttccgg gactttcgct
1381 ttccccctcc ctattgccac ggcggaactc attgccgcct gccttgcccg ctgctggaca
1441 ggggctcggc tgttgggcac tgacaattcc gtggtgttgt cggggaagct gacgtccttt
1501 ccatggctgc tcgcctgtgt tgccaactgg attctgcgcg ggacgtcctt ctgctacgtc
1561 ccttcggccc tcaatccagc ggaccttcct tcccgcggcc tgctgccggt tctgcggcct
1621 cttccgcgtc ttcgccttcg ccctcagacg agtcggatct ccctttgggc cgcctccccg
1681 cctgtttcgc ctcggcgtcc ggtccgtgtt gcttggtctt cacctgtgca gaattgcgaa
1741 ccatggattc caccgtgaac tttgtctcct ggcatgcaaa tcgtcaactt ggcatgccaa
1801 gtaaggacct ttggactcct tatataaaag atcaattatt aactaaatgg gaggagggca
1861 gcattgatcc tagattatca atatttgtat taggaggctg taggcataaa tgcatgcgac
1921 ttctgtaacc atgtatcttt ttcacctgtg ccttgttttt gcctgtgttc catgtcctac
1981 ttttcaagcc tccaagctgt gccttggatg gctttggggc atggacatag atccctataa
2041 agaatttggt tcatcttatc agttgttgaa ttttcttcct ttggacttct ttcctgacct
2101 taatgctttg gtggacactg ctactgcctt gtatgaagaa gagctaacag gtagggaaca
2161 ttgctctccg caccatacag ctattagaca agctttagta tgctgggatg aattaactaa
2221 attgatagct tggatgagct ctaacataac ttctgaacaa gtaagaacaa tcatagtaaa
2281 tcatgtcaat gatacctggg gacttaaggt gagacaaagt ttatggtttc atttgtcatg
2341 tctcactttt ggacaacata cagttcaaga atttttagta agttttggag tatggatcag
2401 aactccagct ccatatagac ctcctaatgc acccattctc tcgactcttc cggaacatac
2461 agtcattagg agaagaggag gtgcaagagc ttctaggtcc cccagaagac gcactccctc
2521 tcctcgcagg agaagatctc aatcaccgcg tcgcagacgc tctcaatctc catctgccaa
2581 ctgctgatct tcaatgggta cataaaacta atgctattac aggtctttac tctaaccaag
2641 ctgctcagtt caatccgcat tggattcaac ctgagtttcc tgaacttcat ttacataatg
2701 atttaattca aaaattgcaa cagtattttg gtcctttgac tataaatgaa aagagaaaat
2761 tgcaattaaa ttttcctgcc agatttttcc ccaaagctac taaatatttc cctttaatta
2821 aaggcataaa aaacaattat cctaattttg ctttagaaca tttctttgct accgcaaatt
2881 atttgtggac tttatgggaa gctggaattt tgtatttaag gaagaatcaa acaactttga
2941 cttttaaagg taaaccatat tcttgggaac acagacagct agtgcaacat aatgggcaac
3001 aacataaaag tcaccttcaa tccagacaaa atagcagcat ggtggcctgc agtgggcact
3061 tattacacaa ccacttatcc tcagaatcag tcagtgtttc aaccaggaat ttatcaaaca
3121 acatctctga taaatcccaa aaatcaacaa gaactggact ctgttcttat aaacagatac
3181 aaacagatag actggaacac ttggcaagga tttcctgtgg atcaaaaatt accattggtc
3241 agcagggatc ctcccccaaa accttatata aatcaatcag ctcaaacttt cgaaatcaaa
3301 cctgggccta taatagttcc cgg
SEQ ID NO: 5
>linker combination
GILE-Xn-L
where X is any amino acid, n is 60 or less
SEQ ID NO: 6
>linker
WLWG
SEQ ID NOS: 7-85 = WHcAg-RSV fusion proteins
>VLP018 (195aa)
MDIDPYKEFGSSYQLLNFLPLDFFPDLNALVDTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIAAGRSPSQSPSQSSANC
>VLP019 (217aa)
MDIDPYKEFGSSYQLLNFLPLDFFPDLNALVDTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIRRRGGARASRSPRRRTPSPRRRRSQSPRRRRSQSPSANC
>VLP020 (230aa)
MGTYMLTNSELLSLINDMPITNDQKKLMSNNVQIVRAGWLWGMDIDPYKEFGSSYQLLNFLPLDFFPDLNALVDTAT
ALYEEELTGREHCSPHHTAIRQALVCWDELTKLIAWMSSNITSEQVRTIIVNHVNDTWGLKVRQSLWFHLSCLTFGQ
HTVQEFLVSFGVWIRTPAPYRPPNAPILSTLPEHTVIRRRGGARASRSPRRRTPSPRRRRSQSPRRRRSQSPSANC
>VLP021 (229aa)
MGTYMLTNSELLSLINDMPITNDQKKLMSNNVQIVRWLWGAMDIDPYKEFGSSYQLLNFLPLDFFPDLNALVDTATA
LYEEELTGREHCSPHHTAIRQALVCWDELTKLIAWMSSNITSEQVRTIIVNHVNDTWGLKVRQSLWFHLSCLTFGQH
TVQEFLVSFGVWIRTPAPYRPPNAPILSTLPEHTVIRRRGGARASRSPRRRTPSPRRRRSQSPRRRRSQSPSANC
>VLP021.1 (208aa)
MGTYMLTNSELLSLINDMPITNDQKKLMSNNVQIVRWLWGAMDIDPYKEFGSSYQLLNFLPLDFFPDLNALVDTATA
LYEEELTGREHCSPHHTAIRQALVCWDELTKLIAWMSSNITSEQVRTIIVNHVNDTWGLKVRQSLWFHLSCLTFGQH
TVQEFLVSFGVWIRTPAPYRPPNAPILSTLPEHTVIAAGRSPSQSPSQSRESQC
>VLP023 (246aa)
MDIDPYKEFGSSYQLLNFLPLDFFPDLNALVDTATALYEEELTGREHCSPHHTAIRQALVCWDELTKLIAWMSSNIT
SGILGGGGSGGGGETYMLTNSELLSLINDMPITNDQKKLMSNNVQIVREGGGGSGGGGLEQVRTIIVNHVNDTWGLK
VRQSLWFHLSCLTFGQHTVQEFLVSFGVWIRTPAPYRPPNAPILSTLPEHTVIRRRGGARASRSPRRRTPSPRRRRS
QSPRRRRSQSPSANC
>VLP025 (191aa)
MDIDPYKEFGSSYQLLNFLPLDFFPDLNALVDTATALYEEELTGREHCSPHHTAIRQALVCWDELSELLSLINDMPI
TNDQKKLMSIIVNHVNDTWGLKVRQSLWFHLSCLTFGQHTVQEFLVSFGVWIRTPAPYRPPNAPILSTLPEHTVIRR
RGGARASRSPRRRTPSPRRRRSQSPRRRRSQSPSANC
>VLP027 (232aa)
MDIDPYKEFGSSYQLLNFLPLDFFPDLNALVDTATALYEEELTGREHCSPHHTAIRQALVCWDELTKLIAWMSSNIT
SGILEVNAGVTTPVSTYMLTNSELLSLINDMPITNDQKKLMSNNLEQVRTIIVNHVNDTWGLKVRQSLWFHLSCLTF
GQHTVQEFLVSFGVWIRTPAPYRPPNAPILSTLPEHTVIRRRGGARASRSPRRRTPSPRRRRSQSPRRRRSQSPSAN
C
>VLP028 (253aa)
MDIDPYKEFGSSYQLLNFLPLDFFPDLNALVDTATALYEEELTGREHCSPHHTAIRQALVCWDELTKLIAWMSSNIT
SEQVRTIIVNHVNDTWGLKVRQSLWFHLSCLTFGQHTVQEFLVSFGVWIRTPAPYRPPNAPILSTLPEHTVIRRRGG
ARASRSPRRGTPSPRRRRSQSPRRRRSQSPSANCDISNIETVIEFQQKNNRLLEITREFSVNAGVTTPVSTYMLTNS
ELLSLINDMPITNDQKKLMSNN
>VLP029 (253aa)
MDIDPYKEFGSSYQLLNFLPLDFFPDLNALVDTATALYEEELTGREHCSPHHTAIRQALVCWDELTKLIAWMSSNIT
SEQVRTIIVNHVNDTWGLKVRQSLWFHLSCLTFGQHTVQEFLVSFGVWIRTPAPYRPPNAPILSTLPEHTVIAAAGG
AAASASPAAATPSPAAARSQSPAAAASQSPSANCDISNIETVIEFQQKNNRLLEITREFSVNAGVTTPVSTYMLTNS
ELLSLINDMPITNDQKKLMSNN
>VLP030 (258aa)
MVSNIETVIEFQQKNNRLLEITREFSVNAGVTTPVSTYMLTNSELLSLINDMPITNDQKKLMSNNWLWGAMDIDPYK
EFGSSYQLLNFLPLDFFPDLNALVDTATALYEEELTGREHCSPHHTAIRQALVCWDELTKLIAWMSSNITSEQVRTI
IVNHVNDTWGLKVRQSLWFHLSCLTFGQHTVQEFLVSFGVWIRTPAPYRPPNAPILSTLPEHTVIRRRGGARASRSP
RRRTPSPRRRRSQSPRRRRSQSPSANC
>VLP031 (237aa)
MVSNIETVIEFQQKNNRLLEITREFSVNAGVTTPVSTYMLTNSELLSLINDMPITNDQKKLMSNNWLWGAMDIDPYK
EFGSSYQLLNFLPLDFFPDLNALVDTATALYEEELTGREHCSPHHTAIRQALVCWDELTKLIAWMSSNITSEQVRTI
IVNHVNDTWGLKVRQSLWFHLSCLTFGQHTVQEFLVSFGVWIRTPAPYRPPNAPILSTLPEHTVIAAGRSPSQSPSQ
SRESQC
>VLP032 (238aa)
MDIDPYKEFGSSYQLLNFLPLDFFPDLNALVDTATALYEEELTGREHCSPHHTAIRQALVCWDELTKLIAWMSSNIT
SGILEVNAGVTTPVSTYMLTNSELLSLINDMPITNDQKKLMSNNVQIVRELEQVRTIIVNHVNDTWGLKVRQSLWFH
LSCLTFGQHTVQEFLVSFGVWIRTPAPYRPPNAPILSTLPEHTVIRRRGGARASRSPRRRTPSPRRRRSQSPRRRRS
QSPSANC
>VLP033 (239aa)
MDIDPYKEFGSSYQLLNFLPLDFFPDLNALVDTATALYEEELTGREHCSPHHTAIRQALVCWDELTKLIAWMSSNIT
SEQVGILEVNAGVTTPVSTYMLTNSELLSLINDMPITNDQKKLMSNNVQIVRELERTIIVNHVNDTWGLKVRQSLWF
HLSCLTFGQHTVQEFLVSFGVWIRTPAPYRPPNAPILSTLPEHTVIRRRGGARASRSPRRRTPSPRRRRSQSPRRRR
SQSPSANC
>VLP034 (253aa)
MDIDPYKEFGSSYQLLNFLPLDFFPDLNALVDTATALYEEELTGREHCSPHHTAIRQALVCWDELTKLIAWMSSNIT
SEQVRTIIVNHVNDTWGLKVRQSLWFHLSCLTFGQHTVQEFLVSFGVWIRTPAPYRPPNAPILSTLPEHTVIAAAGG
AAASASPAAATPSPAAARSQSPRRRRSQSPSANCDISNIETVIEFQQKNNRLLEITREFSVNAGVTTPVSTYMLTNS
ELLSLINDMPITNDQKKLMSNN
>VLP041 (206aa)
MDIDPYKEFGSSYQLLNFLPLDFFPDLNALVDTATALYEEELTGREHCSPHHTAIRQALVCWDELTKLIAWMSSNIT
SGILETYMLTNSELLSLINDMPITNDQKKLMSNNVQIVRELEQVRTIIVNHVNDTWGLKVRQSLWFHLSCLTFGQHT
VQEFLVSFGVWIRTPAPYRPPNAPILSTLPEHTVIAAGRSPSQSPSQSSANC
>VLP042 (229aa)
MDIDPYKEFGSSYQLLNFLPLDFFPDLNALVDTATALYEEELTGREHCSPHHTAIRQALVCWDELTKLIAWMSSNIT
SEQVGILETYMLTNSELLSLINDMPITNDQKKLMSNNVQIVRELERTIIVNHVNDTWGLKVRQSLWFHLSCLTFGQH
TVQEFLVSFGVWIRTPAPYRPPNAPILSTLPEHTVIRRRGGARASRSPRRRTPSPRRRRSQSPRRRRSQSPSANC
>VLP043 (228aa)
MDIDPYKEFGSSYQLLNFLPADFFPAAAVLADTATALYEEELTGREHCSPHHTAIRQALVCWDELTKLIAWMSSNIT
SGILETYMLTNSELLSLINDMPITNDQKKLMSNNVQIVRELEQVRTIIVNHVNDTWGLKVRQSLWFHLSCLTFGQHT
VQEFLVSFGVWIRTPAPYRPPNAPILSTLPEHTVIRRRGGARASRSPRRRTPSPRRRRSQSPRRRRSQSPSANC
>VLP044 (228aa)
MDIDPYKEFGSSYQLLNFLPLDFFPDLNALVDTATALYEEELTGREHCSPHHTAIRQALVCWDELTKLIAWMSSNIT
SGILETYMLTNSELLSLINDMPITNDQKKLMSNNVQIVRELEQVRTIIVNHVNDTWGLKVRQSLWFHLSCLTFGQHT
VQEFLVSFGVWIRTPAPYRPPNAPILSTLPEHTVIRRRGGARASRSPRRRTPSPRRRRSQSPRRRRSQSPSANC
>VLP045 (219aa)
MGNSELLSLINDMPITNDQKKLMSNNWLWGAMDIDPYKEFGSSYQLLNFLPLDFFPDLNALVDTATALYEEELTGRE
HCSPHHTAIRQALVCWDELTKLIAWMSSNITSEQVRTIIVNHVNDTWGLKVRQSLWFHLSCLTFGQHTVQEFLVSFG
VWIRTPAPYRPPNAPILSTLPEHTVIRRRGGARASRSPRRRTPSPRRRRSQSPRRRRSQSPSANC
>VLP046 (198aa)
MGNSELLSLINDMPITNDQKKLMSNNWLWGAMDIDPYKEFGSSYQLLNFLPLDFFPDLNALVDTATALYEEELTGRE
HCSPHHTAIRQALVCWDELTKLIAWMSSNITSEQVRTIIVNHVNDTWGLKVRQSLWFHLSCLTFGQHTVQEFLVSFG
VWIRTPAPYRPPNAPILSTLPEHTVIAAGRSPSQSPSQSRESQC
>VLP047 (215aa)
MDIDPYKEFGSSYQLLNFLPLDFFPDLNALVDTATALYEEELTGREHCSPHHTAIRQALVCWDELTKLIAWMSSNSE
LLSLINDMPITNDQKKLMSNNASSNITSEQVRTIIVNHVNDTWGLKVRQSLWFHLSCLTFGQHTVQEFLVSFGVWIR
TPAPYRPPNAPILSTLPEHTVIRRRGGARASRSPRRRTPSPRRRRSQSPRRRRSQSPSANC
>VLP048 (218aa)
MDIDPYKEFGSSYQLLNFLPLDFFPDLNALVDTATALYEEELTGREHCSPHHTAIRQALVCWDELTKLIAWMSSNIT
SEQVGILENSELLSLINDMPITNDQKKLMSNNLERTIIVNHVNDTWGLKVRQSLWFHLSCLTFGQHTVQEFLVSFGV
WIRTPAPYRPPNAPILSTLPEHTVIRRRGGARASRSPRRRTPSPRRRRSQSPRRRRSQSPSANC
>VLP049 (217aa)
MDIDPYKEFGSSYQLLNFLPADFFPAAAVLADTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIRRRGGARASRSPRRRTPSPRRRRSQSPRRRRSQSPSANC
>VLP050 (238aa)
MDIDPYKEFGSSYQLLNFLPLDFFPDLNALVDTATALYEEELTGREHCSPHHTAIRQALVCWDELTKLIAWMSSNIT
SEQVGILGGGGSGGGGETYMLTNSELLSLINDMPITNDQKKLMSNNVQIVRELERTIIVNHVNDTWGLKVRQSLWFH
LSCLTFGQHTVQEFLVSFGVWIRTPAPYRPPNAPILSTLPEHTVIRRRGGARASRSPRRRTPSPRRRRSQSPRRRRS
QSPSANC
>VLP051 (215aa)
MDIDPYKEFGSSYQLLNFLPLDFFPDLNALVDTATALYEEELTGREHCSPHHTAIRQALVCWDELTKLIAWMSSNII
SGILETYMLTNSELLSLINDMPITNDQKKLMSNNVQIVREGGGGSGGGGLEQVRTIIVNHVNDTWGLKVRQSLWFHL
SCLTFGQHTVQEFLVSFGVWIRTPAPYRPPNAPILSTLPEHTVIAAGRSPSQSPSQSSANC
>VLP052 (237aa)
MDIDPYKEFGSSYQLLNFLPADFFPAAAVLADTATALYEEELTGREHCSPHHTAIRQALVCWDELTKLIAWMSSNIT
SGILETYMLTNSELLSLINDMPITNDQKKLMSNNVQIVREGGGGSGGGGLEQVRTIIVNHVNDTWGLKVRQSLWFHL
SCLTFGQHTVQEFLVSFGVWIRTPAPYRPPNAPILSTLPEHTVIRRRGGARASRSPRRRTPSPRRRRSQSPRRRRSQ
SPSANC
>VLP053 (237aa)
MDIDPYKEFGSSYQLLNFLPLDFFPDLNALVDTATALYEEELTGREHCSPHHTAIRQALVCWDELTKLIAWMSSNIT
SGILGGGGSGGGGETYMLTNSELLSLINDMPITNDQKKLMSNNVQIVRELEQVRTIIVNHVNDTWGLKVRQSLWFHL
SCLTFGQHTVQEFLVSFGVWIRTPAPYRPPNAPILSTLPEHTVIRRRGGARASRSPRRRTPSPRRRRSQSPRRRRSQ
SPSANC
>VLP059 (212aa)
MDIDPYKEFGSSYQLLNFLPLDFFPDLNALVDTATALYEEELTGREHCSPHHTAIRQALVCWDELTKLIAWMSSNIT
SNSELLSLINDMPITNDQKKLMSNNEQVRTIIVNHVNDTWGLKVRQSLWFHLSCLTFGQHTVQEFLVSFGVWIRTPA
PYRPPNAPILSTLPEHTVIRRRGGARASRSPRRRTPSPRRRRSQSPRRRRSQSPSANC
>VLP060 (209aa)
MDIDPYKEFGSSYQLLNFLPLDFFPDLNALVDTATALYEEELTGREHCSPHHTAIRQALVCWDELTKLIAWMSSNIT
SELLSLINDMPITNDQKKLMSNEQVRTIIVNHVNDTWGLKVRQSLWFHLSCLTFGQHTVQEFLVSFGVWIRTPAPYR
PPNAPILSTLPEHTVIRRRGGARASRSPRRRTPSPRRRRSQSPRRRRSQSPSANC
>VLP061 (215aa)
MDIDPYKEFGSSYQLLNFLPLDFFPDLNALVDTATALYEEELTGREHCSPHHTAIRQALVCWDELTKLIAWMSSNSE
LLSLINDMPITNDQKKLMSNNASSAAAAAAAAAIIVNHVNDTWGLKVRQSLWFHLSCLTFGQHTVQEFLVSFGVWIR
TPAPYRPPNAPILSTLPEHTVIRRRGGARASRSPRRRTPSPRRRRSQSPRRRRSQSPSANC
>VLP062 (215aa)
MDIDPYKEFGSSYQLLNFLPLDFFPDLNALVDTATALYEEELTGREHCSPHHTAIRQALVCWDELTKLIAWMSSNSE
LLSLINDMPITNDQKKLMSNNASSELELELELEIIVNHVNDTWGLKVRQSLWFHLSCLTFGQHTVQEFLVSFGVWIR
TPAPYRPPNAPILSTLPEHTVIRRRGGARASRSPRRRTPSPRRRRSQSPRRRRSQSPSANC
>VLP063 (237aa)
MDIDPYKEFGSSYQLLNFLPLDFFPDLNALVDTATALYEEELTGREHCSPHHTAIRQALVCWDELTKLIAWMSSNIT
SGILETYMLTNSELLSLINDMPITNDQKKLMSNNVQIVREGGGGSGGGGLEQVRTIIVNHVNDTWGLKVRQSLWFHL
SCLTFGQHTVQEFLVSFGVWIRTPAPYRPPNAPILSTLPEHTVIRRRGGARASRSPRRRTPSPRRRRSQSPRRRRSQ
SPSANC
>VLP064 (216aa)
MDIDPYKEFGSSYQLLNFLPLDFFPDLNALVDTATALYEEELTGREHCSPHHTAIRQALVCWDELTKLIAWMSSNIT
SGILNSELLSLINDLPASNDQKKLMSNNLEQVRTIIVNHVNDTWGLKVRQSLWFHLSCLTFGQHTVQEFLVSFGVWI
RTPAPYRPPNAPILSTLPEHTVIRRRGGARASRSPRRRTPSPRRRRSQSPRRRRSQSPSANC
>VLP068 (216aa)
MDIDPYKEFGSSYQLLNFLPLDFFPDLNALVDTATALYEEELTGREHCSPHHTAIRQALVCWDELTKLIAWMSSNIT
SGILNSELLSLINDAPAANDQKKLMSNNLEQVRTIIVNHVNDTWGLKVRQSLWFHLSCLTFGQHTVQEFLVSFGVWI
RTPAPYRPPNAPILSTLPEHTVIRRRGGARASRSPRRRTPSPRRRRSQSPRRRRSQSPSANC
>VLP069 (216aa)
MDIDPYKEFGSSYQLLNFLPLDFFPDLNALVDTATALYEEELTGREHCSPHHTAIRQALVCWDELTKLIAWMSSNIT
SGILNSELLSLINDAAAANDQKKLMSNNLEQVRTIIVNHVNDTWGLKVRQSLWFHLSCLTFGQHTVQEFLVSFGVWI
RTPAPYRPPNAPILSTLPEHTVIRRRGGARASRSPRRRTPSPRRRRSQSPRRRRSQSPSANC
>VLP072 (218aa)
MDIDPYKEFGSSYQLLNFLPLDFFPDLNALVDTATALYEEELTGREHCSPHHTAIRQALVCWDELTKLIAWMSSNIG
ILENSELLSLINDMPITNDQKKLMSNNLETSEQVRTIIVNHVNDTWGLKVRQSLWFHLSCLTFGQHTVQEFLVSFGV
WIRTPAPYRPPNAPILSTLPEHTVIRRRGGARASRSPRRRTPSPRRRRSQSPRRRRSQSPSANC
>VLP073 (218aa)
MDIDPYKEFGSSYQLLNFLPLDFFPDLNALVDTATALYEEELTGREHCSPHHTAIRQALVCWDELTKLIAWMSSNIT
SEQVRGILENSELLSLINDMPITNDQKKLMSNNLETIIVNHVNDTWGLKVRQSLWFHLSCLTFGQHTVQEFLVSFGV
WIRTPAPYRPPNAPILSTLPEHTVIRRRGGARASRSPRRRTPSPRRRRSQSPRRRRSQSPSANC
>VLP074 (217aa)
MDIDPYKEFGSSYQLLNFLPLDFFPDLNALVDTATALYEEELTGREHCSPHHTAIRQALVCWDELTKLIAWMSSNIT
SGILANSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIRRRGGARASRSPRRRTPSPRRRRSQSPRRRRSQSPSANC
>VLP075 (216aa)
MDIDPYKEFGSSYQLLNFLPLDFFPDLNALVDTATALYEEELTGREHCSPHHTAIRQALVCWDELTKLIAWMSSNIT
SGILNSELLSLINDMPITNDQKKLMSNNLEQVRTIIVNHVNDTWGLKVRQSLWFHLSCLTFGQHTVQEFLVSFGVWI
RTPAPYRPPNAPILSTLPEHTVIRRRGGARASRSPRRRTPSPRRRRSQSPRRRRSQSPSANC
>VLP076 (220aa)
MDIDPYKEFGSSYQLLNFLPLDFFPDLNALVDTATALYEEELTGREHCSPHHTAIRQALVCWDELTKLIAWMSSNIT
SGILEENSELLSLINDMPITNDQKKLMSNNEELEQVRTIIVNHVNDTWGLKVRQSLWFHLSCLTFGQHTVQEFLVSF
GVWIRTPAPYRPPNAPILSTLPEHTVIRRRGGARASRSPRRRTPSPRRRRSQSPRRRRSQSPSANC
>VLP077 (217aa)
MDIDPYKEFGSSYQLLNFLPLDFFPDLNALVDTATALYEEELTGREHCSPHHTAIRQALVCWDELTKLIAWMSSNIT
SGILEIKQSLMTDSLSNPNNLNNDIKLEMLEQVRTIIVNHVNDTWGLKVRQSLWFHLSCLTFGQHTVQEFLVSFGVW
IRTPAPYRPPNAPILSTLPEHTVIRRRGGARASRSPRRRTPSPRRRRSQSPRRRRSQSPSANC
>VLP078 (217aa)
MDIDPYKEFGSSYQLLNFLPLDFFPDLNALVDTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIAAAGGAAASASPAAATPSPAAARSQSPAAAASQSPSANC
>VLP079 (217aa)
MDIDPYKEFGSSYQLLNFLPLDFFPDLNALVDTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIAAAGGAAASASPAAATPSPAAARSQSPRRRRSQSPSANC
>VLP080 (212aa)
MDIDPYKEFGSSYQLLNFLPLDFFPDLNALVDTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIDIDYINKLQNTITSDWTPCTVSRRRRSQSPRRRR
>VLP081 (204aa)
MDIDPYKEFGSSYQLLNFLPLDFFPDLNALVDTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIDIDYINKLQNTITSDWTPCTVSRRRR
>VLP087 (191aa)
MDIDPYKEFGSSYQLLNFLPLDFFPDLNALVDTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIRRGGARASQSANC
>VLP088 (217aa)
MDIDPYKEFGSSYQLLNFLPLDFFPDLNALVDTATALYEEELTGREHCSPHHTAIRQALVCWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPPPPILSTLPEHTVIRRRGGARASRSPRRRTPSPRRRRSQSPRRRRSQSPSANC
>VLP089 (220aa)
MDIDPYKEFGSSYQLLNFLPLDFFPDLNALVDTATALYEEELTGREHCSPHHTAIRQALVCWDELTKLIAWMSSNIT
SGILPENSELLSLINDMPITNDQKKLMSNNPELEQVRTIIVNHVNDTWGLKVRQSLWFHLSCLTFGQHTVQEFLVSF
GVWIRTPAPYRPPNAPILSTLPEHTVIRRRGGARASRSPRRRTPSPRRRRSQSPRRRRSQSPSANC
>VLP090 (216aa)
MDIDPYKEFGSSYQLLNFLPLDFFPDLNALVDTATALYEEELTGREHCSPHHTAIRQALVCWDELTKLIAWMSSNIT
SGILNSELLSLIHDMPITNDQKKLMSNNLEQVRTIIVNHVNDTWGLKVRQSLWFHLSCLTFGQHTVQEFLVSFGVWI
RTPAPYRPPNAPILSTLPEHTVIRRRGGARASRSPRRRTPSPRRRRSQSPRRRRSQSPSANC
>VLP091 (216aa)
MDIDPYKEFGSSYQLLNFLPLDFFPDLNALVDTATALYEEELTGREHCSPHHTAIRQALVCWDELTKLIAWMSSNIT
SGILNSELLSLINDMPAANDQKKLMSNNLEQVRTIIVNHVNDTWGLKVRQSLWFHLSCLTFGQHTVQEFLVSFGVWI
RTPAPYRPPNAPILSTLPEHTVIRRRGGARASRSPRRRTPSPRRRRSQSPRRRRSQSPSANC
>VLP092 (216aa)
MDIDPYKEFGSSYQLLNFLPLDFFPDLNALVDTATALYEEELTGREHCSPHHTAIRQALVCWDELTKLIAWMSSNIT
SGILNSELLSLINDAPASNDQKKLMSNNLEQVRTIIVNHVNDTWGLKVRQSLWFHLSCLTFGQHTVQEFLVSFGVWI
RTPAPYRPPNAPILSTLPEHTVIRRRGGARASRSPRRRTPSPRRRRSQSPRRRRSQSPSANC
>VLP093 (217aa)
MDIDPYKEFGSSYQLLNFLPLDFFPDLNALVDTATALYEEELTGREHCSPHHTAIRQALVSWDELTKLIAWMSSNIT
SGILENSELLSLINDMPITNDQKKLMSNNLEQVRTIIVNHVNDTWGLKVRQSLWFHLSCLTFGQHTVQEFLVSFGVW
IRTPAPYRPPNAPILSTLPEHTVIRRRGGARASRSPRRRTPSPRRRRSQSPRRRRSQSPSANC
>VLP094 (213aa)
MDIDPYKEFGSSYQLLNFLPLDFFPDLNALVDTATALYEEELTGREHCSPHHTAIRQALVCWDELTKLIAWMSSNIT
STNSELLSLINDMPITNDQKKLMSNNEQVRTIIVNHVNDTWGLKVRQSLWFHLSCLTFGQHTVQEFLVSFGVWIRTP
APYRPPNAPILSTLPEHTVIRRRGGARASRSPRRRTPSPRRRRSQSPRRRRSQSPSANC
>VLP095 (214aa)
MDIDPYKEFGSSYQLLNFLPLDFFPDLNALVDTATALYEEELTGREHCSPHHTAIRQALVCWDELTKLIAWMSSNIT
SLTNSELLSLINDMPITNDQKKLMSNNEQVRTIIVNHVNDTWGLKVRQSLWFHLSCLTFGQHTVQEFLVSFGVWIRT
PAPYRPPNAPILSTLPEHTVIRRRGGARASRSPRRRTPSPRRRRSQSPRRRRSQSPSANC
>VLP096 (215aa)
MDIDPYKEFGSSYQLLNFLPLDFFPDLNALVDTATALYEEELTGREHCSPHHTAIRQALVCWDELTKLIAWMSSNIT
SMLTNSELLSLINDMPITNDQKKLMSNNEQVRTIIVNHVNDTWGLKVRQSLWFHLSCLTFGQHTVQEFLVSFGVWIR
TPAPYRPPNAPILSTLPEHTVIRRRGGARASRSPRRRTPSPRRRRSQSPRRRRSQSPSANC
>VLP097 (213aa)
MDIDPYKEFGSSYQLLNFLPLDFFPDLNALVDTATALYEEELTGREHCSPHHTAIRQALVCWDELTKLIAWMSSNIT
SNSELLSLINDMPITNDQKKLMSNNVEQVRTIIVNHVNDTWGLKVRQSLWFHLSCLTFGQHTVQEFLVSFGVWIRTP
APYRPPNAPILSTLPEHTVIRRRGGARASRSPRRRTPSPRRRRSQSPRRRRSQSPSANC
>VLP098 (214aa)
MDIDPYKEFGSSYQLLNFLPLDFFPDLNALVDTATALYEEELTGREHCSPHHTAIRQALVCWDELTKLIAWMSSNIT
SNSELLSLINDMPITNDQKKLMSNNVQEQVRTIIVNHVNDTWGLKVRQSLWFHLSCLTFGQHTVQEFLVSFGVWIRT
PAPYRPPNAPILSTLPEHTVIRRRGGARASRSPRRRTPSPRRRRSQSPRRRRSQSPSANC
>VLP099 (215aa)
MDIDPYKEFGSSYQLLNFLPLDFFPDLNALVDTATALYEEELTGREHCSPHHTAIRQALVCWDELTKLIAWMSSNIT
SNSELLSLINDMPITNDQKKLMSNNVQIEQVRTIIVNHVNDTWGLKVRQSLWFHLSCLTFGQHTVQEFLVSFGVWIR
TPAPYRPPNAPILSTLPEHTVIRRRGGARASRSPRRRTPSPRRRRSQSPRRRRSQSPSANC
>VLP111 (207aa)
MDIDPYKEFGSSYQLLNFLPLDFFPDLNALVDTATALYEEELTGREHCSPHHTAIRQALVCWDELTKLIAWMSSNSE
LLSLINDMPITNDQKKLMSNNQVRTIIVNHVNDTWGLKVRQSLWFHLSCLTFGQHTVQEFLVSFGVWIRTPAPYRPP
NAPILSTLPEHTVIRRRGGARASRSPRRRTPSPRRRRSQSPRRRRSQSPSANC
>VLP112 (208aa)
MDIDPYKEFGSSYQLLNFLPLDFFPDLNALVDTATALYEEELTGREHCSPHHTAIRQALVCWDELTKLIAWMSSTNS
ELLSLINDMPITNDQKKLMSNNQVRTIIVNHVNDTWGLKVRQSLWFHLSCLTFGQHTVQEFLVSFGVWIRTPAPYRP
PNAPILSTLPEHTVIRRRGGARASRSPRRRTPSPRRRRSQSPRRRRSQSPSANC
>VLP113 (208aa)
MDIDPYKEFGSSYQLLNFLPLDFFPDLNALVDTATALYEEELTGREHCSPHHTAIRQALVCWDELTKLIAWMSSNSE
LLSLINDMPITNDQKKLMSNNVQVRTIIVNHVNDTWGLKVRQSLWFHLSCLTFGQHTVQEFLVSFGVWIRTPAPYRP
PNAPILSTLPEHTVIRRRGGARASRSPRRRTPSPRRRRSQSPRRRRSQSPSANC
>VLP114 (209aa)
MDIDPYKEFGSSYQLLNFLPLDFFPDLNALVDTATALYEEELTGREHCSPHHTAIRQALVCWDELTKLIAWMSSTNS
ELLSLINDMPITNDQKKLMSNNVQVRTIIVNHVNDTWGLKVRQSLWFHLSCLTFGQHTVQEFLVSFGVWIRTPAPYR
PPNAPILSTLPEHTVIRRRGGARASRSPRRRTPSPRRRRSQSPRRRRSQSPSANC
>VLP120 (188aa)
MDIDPYKEFGSSYQLLNFLPLDFFPDLNALVDTATALYEEELTGREHCSPHHTAIRQALVCNSELLSLINDMPITND
QKKLMSNNVNHVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
>VLP121 (188aa)
MDIDPYKEFGSSYQLLNFLPLDFFPDLNALVDTATALYEEELTGREHCSPHHTAIRQALVCWNSELLSLINDMPITN
DQKKLMSNNNHVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
>VLP122 (188aa)
MDIDPYKEFGSSYQLLNFLPLDFFPDLNALVDTATALYEEELTGREHCSPHHTAIRQALVCWDNSELLSLINDMPIT
NDQKKLMSNNHVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
>VLP123 (188aa)
MDIDPYKEFGSSYQLLNFLPLDFFPDLNALVDTATALYEEELTGREHCSPHHTAIRQALVCWDNSELLSLINDMPIT
NDQKKLMSNNHVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
>VLP124 (186aa)
MDIDPYKEFGSSYQLLNFLPLDFFPDLNALVDTATALYEEELTGREHCSPHHTAIRQALVCWDELLSLINDMPITND
QKKLMSNNHVNDTWGLKVRQSLWFHLSCLTFGQHTVQEFLVSFGVWIRTPAPYRPPNAPILSTLPEHTVIRRRGGAR
ASRSPRRRTPSPRRRRSQSPRRRRSQSPSANC
>VLP125 (188aa)
MDIDPYKEFGSSYQLLNFLPLDFFPDLNALVDTATALYEEELTGREHCSPHHTAIRQALVSNSELLSLINDMPITND
QKKLMSNNVNHVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
>VLP126 (188aa)
MDIDPYKEFGSSYQLLNFLPLDFFPDLNALVDTATALYEEELTGREHCSPHHTAIRQALVSWNSELLSLINDMPITN
DQKKLMSNNNHVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
>VLP127 (188aa)
MDIDPYKEFGSSYQLLNFLPLDFFPDLNALVDTATALYEEELTGREHCSPHHTAIRQALVSWDNSELLSLINDMPIT
NDQKKLMSNNHVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
>VLP128 (188aa)
MDIDPYKEFGSSYQLLNFLPLDFFPDLNALVDTATALYEEELTGREHCSPHHTAIRQALVSWDENSELLSLINDMPI
TNDQKKLMSNNVNDTWGLKVRQSLWFHLSCLTFGQHTVQEFLVSFGVWIRTPAPYRPPNAPILSTLPEHTVIRRRGG
ARASRSPRRRTPSPRRRRSQSPRRRRSQSPSANC
>VLP129 (186aa)
MDIDPYKEFGSSYQLLNFLPLDFFPDLNALVDTATALYEEELTGREHCSPHHTAIRQALVSWDELLSLINDMPITND
QKKLMSNNHVNDTWGLKVRQSLWFHLSCLTFGQHTVQEFLVSFGVWIRTPAPYRPPNAPILSTLPEHTVIRRRGGAR
ASRSPRRRTPSPRRRRSQSPRRRRSQSPSANC
>VLP130 (215aa)
MDIDPYKEFGSSYQLLNFLPLDFFPDLNALVDTATALYEEELTGREHCSPHHTAIRQALVCWDELTKLIAWMSSNIT
SGINSELLSLINDMPITNDQKKLMSNNLEQVRTIIVNHVNDTWGLKVRQSLWFHLSCLTFGQHTVQEFLVSFGVWIR
TPAPYRPPNAPILSTLPEHTVIRRRGGARASRSPRRRTPSPRRRRSQSPRRRRSQSPSANC
>VLP131 (214aa)
MDIDPYKEFGSSYQLLNFLPLDFFPDLNALVDTATALYEEELTGREHCSPHHTAIRQALVCWDELTKLIAWMSSNIT
SGNSELLSLINDMPITNDQKKLMSNNLEQVRTIIVNHVNDTWGLKVRQSLWFHLSCLTFGQHTVQEFLVSFGVWIRT
PAPYRPPNAPILSTLPEHTVIRRRGGARASRSPRRRTPSPRRRRSQSPRRRRSQSPSANC
>VLP132 (214aa)
MDIDPYKEFGSSYQLLNFLPLDFFPDLNALVDTATALYEEELTGREHCSPHHTAIRQALVCWDELTKLIAWMSSNIT
SINSELLSLINDMPITNDQKKLMSNNLEQVRTIIVNHVNDTWGLKVRQSLWFHLSCLTFGQHTVQEFLVSFGVWIRT
PAPYRPPNAPILSTLPEHTVIRRRGGARASRSPRRRTPSPRRRRSQSPRRRRSQSPSANC
>VLP133 (215aa)
MDIDPYKEFGSSYQLLNFLPLDFFPDLNALVDTATALYEEELTGREHCSPHHTAIRQALVCWDELTKLIAWMSSNIT
SILNSELLSLINDMPITNDQKKLMSNNLEQVRTIIVNHVNDTWGLKVRQSLWFHLSCLTFGQHTVQEFLVSFGVWIR
TPAPYRPPNAPILSTLPEHTVIRRRGGARASRSPRRRTPSPRRRRSQSPRRRRSQSPSANC
>VLP134 (215aa)
MDIDPYKEFGSSYQLLNFLPLDFFPDLNALVDTATALYEEELTGREHCSPHHTAIRQALVCWDELTKLIAWMSSNIT
SGILNSELLSLINDMPITNDQKKLMSNNEQVRTIIVNHVNDTWGLKVRQSLWFHLSCLTFGQHTVQEFLVSFGVWIR
TPAPYRPPNAPILSTLPEHTVIRRRGGARASRSPRRRTPSPRRRRSQSPRRRRSQSPSANC
>VLP135 (216aa)
MDIDPYKEFGSSYQLLNFLPLDFFPDLNALVDTATALYEEELTGREHCSPHHTAIRQALVCWDELTKLIAWMSSNIT
SGILNSELLSLINDMPITNDQKKLMSNNVEQVRTIIVNHVNDTWGLKVRQSLWFHLSCLTFGQHTVQEFLVSFGVWI
RTPAPYRPPNAPILSTLPEHTVIRRRGGARASRSPRRRTPSPRRRRSQSPRRRRSQSPSANC
```

SEQ ID NOS:86-114=RSV F polypeptide inserts of Table 1-B.

We claim:

1. An antigenic composition comprising a hybrid woodchuck hepadnavirus core antigen, wherein the hybrid core antigen is a fusion protein comprising a respiratory syncytial virus (RSV) F polypeptide and a woodchuck hepadnavirus core antigen, and wherein said fusion protein is capable of assembling as a hybrid virus-like particle (VLP).

2. A vaccine comprising the antigenic composition of claim 1, and an adjuvant.

3. A method for eliciting a RSV-reactive antibody response, comprising administering to a mammal an effective amount of the antigenic composition of claim 1.

4. A method for reducing RSV infection or preventing RSV disease in a mammal in need thereof, comprising administering to the mammal an effective amount of the vaccine of claim 2 accordingly to a suitable vaccine regimen comprising an initial immunization and one or more subsequent immunizations.

5. A method for screening anti-RSV antibodies comprising:
a) measuring binding of an antibody or fragment thereof to a hybrid woodchuck hepadnavirus core antigen, wherein the hybrid core antigen is a fusion protein comprising a respiratory syncytial virus (RSV) F polypeptide and a woodchuck hepadnavirus core antigen, and wherein said fusion protein assembles as a hybrid virus-like particle (VLP); and
b) measuring binding of the antibody or fragment thereof to a woodchuck hepadnavirus VLP devoid of the RSV F polypeptide; and
c) determining that the antibody or fragment thereof is specific for the RSV F polypeptide when the antibody or fragment thereof binds to the hybrid VLP but not the woodchuck hepadnavirus VLP devoid of the RSV F polypeptide.
